(19) United States
(12) Patent Application Publication
(10) Pub. No.: US 2026/0042723 A1
(43) Pub. Date: Feb. 12, 2026

(54) PURIFICATION TECHNOLOGIES

(71) Applicant: ZYMOCHEM, INC., San Leandro, CA (US)

(72) Inventors: Tatsuya Shigehiro, Sakura (JP); Masanori Miyamoto, Sakura (JP); Gregory Martin Bontemps, Emeryville, CA (US)

(21) Appl. No.: 18/996,738

(22) PCT Filed: Jul. 20, 2023
(86) PCT No.: PCT/US2023/028279

§ 371 Date: Jan. 17, 2025

Related U.S. Application Data
(60) Provisional application No. 63/390,947, filed on Jul. 20, 2022.
(60) Provisional application No. 63/446,783, filed on Feb. 17, 2023.

(51) Int. Cl. C07C 29/76; B01D 3/10; B01D 15/36; B01J 39/05; B01J 39/07; B01J 39/20; B01J 41/05; B01J 41/07; B01J 41/14; B01J 47/016; C07C 31/20; C12P 7/18

(52) U.S. Cl. CPC C07C 29/76; B01D 3/10; B01D 15/362; B01D 15/363; B01J 39/05; B01J 39/07; B01J 39/20; B01J 41/05; B01J 41/07; B01J 41/14; B01J 47/016; C07C 31/20; C12P 7/18

(57) ABSTRACT

The present invention provides various technologies relating to purification of certain glycol compounds, and in particular for purification of such compounds when present in an aqueous composition such as a fermentation culture or composition prepared therefrom, where such fermentation culture comprises cell(s) (e.g., microbial cells) that produce or have produced (e.g., having been engineered to produce) the relevant glycol compound(s).

### CROSS REFERENCE TO RELATED APPLICATIONS

This application claims priority to United States Provisional Application Nos. 63/390,947, filed Jul. 20, 2022, and 63/446,783 filed Feb. 17, 2023, the entire of each of which is incorporated herein by reference.

### BACKGROUND

Glycol compounds have a variety of uses and applications.

### SUMMARY

The present invention provides various technologies relating to purification of certain glycol compounds, and in particular for purification of such compounds when present in an aqueous composition. In some embodiments, provided technologies are useful to purify a glycol compound from an aqueous composition that include cell(s) (e.g., microbial cell(s)) and/or component(s) thereof.

In some embodiments, provided are ion exchange (IEX) technologies. In some embodiments, provided technologies are demonstrated to successfully purify relevant glycol compound(s) from aqueous composition(s) with less loss of glycol compound due to IEX resin adsorption than is observed with appropriate relevant reference process.

In some embodiments, provided are filtration technologies. In some embodiments, provided technologies are demonstrated to successfully separate insoluble mass and macromolecular contaminants from a desired aqueous glycol compound composition (e.g., one that may contain cell(s), such as microbial cell(s) and/or component(s) thereof).

In some embodiments, the present disclosure provides combinations of technological improvements (e.g., filtration technologies and/or IEX technologies) that together achieve particularly effective purification of glycol compound preparations, especially from aqueous sources or compositions.

Technologies provided by the present disclosure solve certain problems that can be encountered when endeavoring to purify glycol compound(s), in particular from aqueous compositions. The present disclosure furthermore provides an insight that provided technologies are particularly useful for purification of certain glycol compounds as described herein (e.g., “long chain” glycol compounds, such as fatty alcohol compound(s) having at least 5 carbon atoms). Moreover, the present disclosure provides an insight that provided technologies are particularly useful when isolating such compounds from aqueous compositions, and particularly from those containing greater than about 50% water and/or from those containing cell(s) (e.g., microbial cell(s)) and/or components thereof.

The present disclosure identifies the source of a particular problem associated with IEX purification of glycol compound(s) as discussed herein (e.g., fatty alcohol compound(s) having at least 5 carbon atoms), particularly from aqueous composition(s), that is, extent to which the relevant compound is retained on an IEX resin. For example, the present disclosure identifies the source of a problem with many typical IEX technologies, with respect to their application to purification of glycol compounds as described herein. For example, the present disclosure provides a surprising teaching that improved results (e.g., less resin retention) can be achieved through use of a hydrophilic resin (e.g., than might be achieved with a typical hydrophobic resin such as a polystyrene support).

Among other things, the present disclosure demonstrates that surprisingly, pre-masking and/or post-recovering can significantly reduce(s) loss of glycol compounds. In some embodiments, unexpected reduction is achieved even when resins whose base polymers are hydrophobic are utilized.

The present disclosure appreciates that level of resin retention may present particular challenges when larger scale of broth volume (e.g., in a range of about 10 kL to about 1000 kL, or in some embodiments about 20 kL to about 80 kL, or even about 30 kL to about 70 kL) purification is desired. In some embodiments, provided technologies are performed at scales within a range of about 10 kL to about 1000 kL.

The present invention identifies the source of problem(s) associated with purifying high order (e.g., containing more than 5 carbon atoms, such as 5-12 carbon atoms) glycol compounds from aqueous preparations containing other ions and fermentation cellular matter.

Among other things, the present invention identifies challenges in separating hydrophilic and hydrophobic species from each other, including specifically in the context of purifying high order (e.g., containing more than 5 carbon atoms, such as 5-12 carbon atoms) glycol compounds from aqueous preparations containing other ions and fermentation cellular matter.

Among other things, in some embodiments, the present invention provides novel and improved technologies for separating ions from aqueous solution(s).

In some embodiments, the present disclosure provides a glycol compound preparation the manufacturing of which comprises one or more provided methods. In some embodiments, the present disclosure provides methods for manufacturing a product using such a glycol compound preparation. In some embodiments, a product is a polymer. In some embodiments, a product is a solvent. In some embodiments, a product is an acrylate. In some embodiments, a product is polyurethane. In some embodiments, a product is a polyester. In some embodiments, a product is an adhesive. In some embodiments, a product is a plasticizer.

In some embodiments, the present disclosure provides a method of characterizing a resin as suitable for purifying an aqueous initial preparation comprising a glycol compound, comprising determining whether the particle size, cross-linking density, functional group identity, functional group density, and hydrophilicity of the resin is within the appropriate or desired range or having the appropriate or desired identity. In some embodiments, the present disclosure provides a method of characterizing a resin as suitable for purifying an aqueous initial preparation comprising a glycol compound, comprising determining whether loss of the glycol compound is acceptable when the resin is utilized for ion exchange according to one or more methods and/or protocols described herein. In some embodiments, the present disclosure provides a method of characterizing a resin as suitable for purifying an aqueous initial preparation comprising a glycol compound, comprising determining whether loss of the glycol compound is lower than a hydrophobic resin when the resin is utilized for ion exchange under comparable conditions with a hydrophobic resin.

In some embodiments, the present disclosure provides high purity preparations of glycol compounds. In some embodiments, the present disclosure provides purified preparations of glycol compounds in which preparations various ions and/or impurities are independently present in an amount that is about or at least about 2, 3, 4, 5, 6, 7, 8, 9, 10, 11, 12, 13, 14, 15, 16, 17, 18, 19, 20, 30, 40, 50, 60, 70, 80, 90, 100, 200, 500, or 1000 fold less than that present in the aqueous initial preparation from which the glycol compound is purified.

In some embodiments, a provided method comprises decolorization and/or deodorization. In some embodiments, a HDO composition, e.g., a HDO preparation, is provided as a colorless liquid (e.g., a HDO solution). In some embodiments, a HDO composition, e.g., a HDO preparation, is provided as a colorless solid.

### BRIEF DESCRIPTION OF THE DRAWING

FIG. 1. Schematic flow diagram showing a useful sequence of processing steps from fermentation to a purified glycol compound as an example.

### DEFINITIONS

Associated: Two events or entities are “associated” with one another, as that term is used herein, if the presence, level, degree, type and/or form of one is correlated with that of the other. For example, a particular entity (e.g., polypeptide, genetic signature, metabolite, microbe, etc) is considered to be associated with a particular disease, disorder, or condition, if its presence, level and/or form correlates with incidence of, susceptibility to, severity of, stage of, etc the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

Comparable: As used herein, the term “comparable” refers to two or more agents, entities, situations, sets of conditions, etc., that may not be identical to one another but that are sufficiently similar to permit comparison therebetween so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Cross-linking: As used herein, the terms “cross-linking”, “cross-linked”, “cross-link”, or grammatical equivalents thereof, refer to inter-polymer strand covalent linking of the polymer support structure backbone. Cross-linking affects many critical macroscopic and microscopic parameters of a polymer or resin, including but not limited to particle size, equivalents per unit of resin, mechanical strength and resistance to deformation, hydrophilicity and hydrophobicity, and number of regeneration cycles, and total lifetime.

Equivalents per unit of resin: Those familiar with the art will appreciate that resins are composite mixtures forming a union of functionalities and chemical identities. The total number of sites available for ion exchange may be determined before or after converting the resin by chemical regeneration techniques to a given ionic form (e.g., exchanging an anion exchange resin's chloride ion for a hydroxide ion). An ion is then chemically removed from a measured quantity from a resin and quantitatively determined in solution by conventional analytical methods. Total ion capacity may be expressed on a dry weight, wet weight, wet volume, or dry volume basis. Water uptake of a resin and therefore its wet weight and wet volume capacities are dependent on the nature of the polymer support structure as well as on the environment in which the sample is placed. Standard ways of expressing the number of charged functional groups available for exchange may be written as eq/L (equivalents per liter) or eq/g (equivalents per gram), including transformed alternatives, such as meq/g (milli-equivalents per gram).

Glycol compound: The term “glycol compound”, as used herein, refers to a compound having the structure of L-(OH)ₙ wherein n is 2 or 3 and L is bivalent or trivalent C₂₋₁₅ (e.g., C₂, C₃, C₄, C₅, C₆, C₇, C₈, C₉, C₁₀, C₁₁, C₁₂, C₁₃, C₁₄ or C₁₅) aliphatic moiety. In some embodiments, n is 2. In some embodiments, n is 3. In some embodiments, L is saturated. In some embodiments, L is unsaturated. In some embodiments, L is linear. In some embodiments, L is branched. In some embodiments, L is a C₂₋₁₂ aliphatic moiety. In some embodiments, L is a C₂₋₁₀ aliphatic moiety. In some embodiments, L is a C₅₋₁₂ aliphatic moiety. In some embodiments, L is a C₅₋₁₀ aliphatic moiety. In some embodiments, L is a C₅₋₈ aliphatic moiety. In some embodiments, a glycol compound is HO(CH₂)₂OH. In some embodiments, a glycol compound is HO(CH₂)₃OH. In some embodiments, a glycol compound is HO(CH₂)₄OH. In some embodiments, a glycol compound is HO(CH₂)₅OH. In some embodiments, a glycol compound is HO(CH₂)₆OH. In some embodiments, a glycol compound is HO(CH₂)₇OH. In some embodiments, a glycol compound is HO(CH₂)₈OH. In many embodiments of a glycol compound, hydroxyl groups are attached to different carbon atoms.

Improve, increase, inhibit or reduce: As used herein, terms such as “improve”, “increase”, “inhibit’, “reduce”, or grammatical equivalents thereof, indicate values that are relative to a baseline or other reference measurement. In some embodiments, an appropriate reference measurement may be or comprise a measurement in a particular system (e.g., in a single individual) under otherwise comparable conditions absent presence of (e.g., prior to and/or after) a particular agent or treatment, or in presence of an appropriate comparable reference agent. In some embodiments, an appropriate reference measurement may be or comprise a measurement in comparable system known or expected to respond in a particular way, in presence of the relevant agent or treatment.

Ion exchange resin: Ion exchange is a reversible interchange of ions between a solid (ion exchange resin) and a liquid. An ion exchange resin may comprise a cross-linked polymer support structure with a distribution of ionic active sites throughout. Ion exchange resins may have utility in chemical synthesis, fermentation, biocatalysis, medical research, food processing, mining, agriculture and a variety of other areas.

Matrix: As used herein, the term “matrix”, refers to the identity of a polymer or copolymer as derived from a set of monomer(s) (e.g., polystyrene is derived from styrene by way of a polymerization reaction). In some embodiments, a polymer matrix is a polymer support structure.

Particle size: As used herein, the term “particle size”, refers to the longest dimension of a particle. In some embodiments, a population of particles is characterized by an average size. In some embodiments, a particle may be substantially spherical (e.g., so that its longest dimension may be its diameter). In some embodiments, an ion exchange resin is characterized by its average particle size; in some embodiments, an ion exchange resin useful in accordance with the present disclosure may have an average particle size (e.g., diameter) within a range of about 100 μm to 1500 μm.

Porosity: As used herein, the term “porosity”, is used in reference to voids present in or on a resin or matrix (e.g., an ion exchange resin), such as in or on particle(s) of such resin or matrix. Typically, conditions under which a resin or matrix is formed (e.g., polymerization conditions under which a polymer matrix is formed) determine its porosity. Those skilled in the art are aware that aspects of polymerization conditions (e.g., temperature, pressure, porosity) can impact porosity of a polymer matrix. A matrix's porosity may impact or determine, for example, what size molecule, complex, or ion may enter the matrix's inner structure; at what rate a molecule, complex, or ion might diffuse into or through such matrix, kinetic profile(s) of any exchange process that may occur in or on a matrix. Alternatively or additionally, there may be a relationship between equilibrium properties of swelling, that is, enlargement of the resin's volume based on contacted solvent and/or initial aqueous preparation(s), and ionic selectivity that is, not all ions behave the same toward a resin, and the swelling characteristics may alter ionic affinity further. For example, conventional gel type ion exchange resin may be prepared by co-polymerizing styrene and divinylbenzene (DVB). Gel resins typically exhibit microporosity with pore volumes between 1 nm (10 Å) and 1.5 nm (15 Å). Macroporous ion exchange resins have pores of a considerably larger size, with pore diameters of 10 nm (100 Å) to over 100 nm (1000 Å). Macroporous polymers are generally highly cross-linked and therefore exhibit little volume change (swelling) when contacted with solvents, including water or aqueous preparations.

Post-recovering: As used herein, the term “post-recovering” refers to contacting a resin with a suitable solvent system, e.g., water (e.g., deionized water), an alcohol (e.g., methanol and/or ethanol), an organic solvent (e.g., acetone and/or tetrahydrofuran), or a mixture of two or more such solvents, to recover from an ion exchange resin a chemical entity of interest, e.g., a purified chemical entity of interested described herein (e.g., in some embodiments, 1,6-hexanediol (HDO)).

Pre-conditioning: As used herein, the term “pre-conditioning” refers to a step performed on an ion exchange resin in advance of contacting a sample of interest to the resin. In some embodiments, pre-conditioning involves contacting an ion exchange resin with a solution containing an ion of alternate identity, and/or regenerating an ion exchange resin to an ion of preferred identity.

Pre-masking: As used herein, the term “pre-mask” refers to contacting an ion exchange resin with a purification target (e.g., an entity of interest such as HDO), which in various embodiments is supplied in a solution. In some embodiments, pre-masking is performed on an ion exchange resin before the resin is contacted with a composition comprising a purification target, which composition typically comprises various impurities (e.g., a broth comprising a purification target). Typically, a composition of a purification target, e.g., a solution of a purification target, is pure, or of higher purity, compared to a composition (e.g., a broth) from which a purification target is to be purified from. In some embodiments, a composition of a purification target for pre-masking is a solution of the purification target in water or in a suitable buffer. In some embodiments, such a buffer is free of impurities that are typically removed during purification using an ion exchange resin.

Purify: As used herein, the term “purify” and its grammatical relatives (e.g., “purification”, etc), refers to a process of enriching a composition for a particular entity of interest. In some embodiments, purification may involve removal of one or more components from a composition (e.g., a mixture) that contains the entity of interest. Alternatively or additionally, in some embodiments, purification may involve increasing a relative amount of an entity of interest, e.g., relative to one or more other components of a composition that includes the entity. In some embodiments, purification may involve one or more physical of chemical operations. For example, in some embodiments, purification may involve one or more of evaporation, distillation, filtration, centrifugation, liquid-liquid phase separation, solid-liquid phase separation, electrostatic fractionation, or solid-mobile phase chromatography.

Reference: As used herein describes a standard or control relative to which a comparison is performed. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Retentate: Retentate is a fraction of solution or mixture that is retained by a filter or a porous membrane, for example, in various embodiments in this disclosure the term refers to solution or slurry retained during all tangential flow filtration (TFF) unit operations.

Sample: As used herein, the term “sample” typically refers to an aliquot of material obtained or derived from a source of interest, as described herein. In some embodiments, a source of interest is a chemical, biological or environmental source. In some embodiments, a source of interest may be or comprise a cell or an organism, such as a microbe, a plant, or an animal (e.g., a human). In some embodiments, a sample is a “primary sample” obtained directly from a source of interest by any appropriate means. In some embodiments, as will be clear from context, the term “sample” refers to a preparation that is obtained by processing (e.g., by removing one or more components of and/or by adding one or more agents to) a primary sample, for example by filtering (e.g., using a semi-permeable membrane), and/or by adsorbing to an ion exchange resin (e.g., as in ion exchange chromatography).

Small molecule: As used herein, the term “small molecule” means a low molecular weight organic and/or inorganic compound. In general, a “small molecule” is a molecule that is less than about 5 kilodaltons (kD) in size. In some embodiments, a small molecule is less than about 4 kD, 3 kD, about 2 kD, or about 1 kD. In some embodiments, the small molecule is less than about 800 daltons (D), about 600 D, about 500 D, about 400 D, about 300 D, about 200 D, or about 100 D. In some embodiments, a small molecule is less than about 2000 g/mol, less than about 1500 g/mol, less than about 1000 g/mol, less than about 800 g/mol, or less than about 500 g/mol. In some embodiments, a small molecule is not a polymer. In some embodiments, a small molecule does not include a polymeric moiety. In some embodiments, a particular small molecule compound may be provided and/or utilized in a salt form (e.g., in an acid-addition or base-addition salt form, depending on the compound); in some such embodiments, the salt form may be a pharmaceutically acceptable salt form. In some embodiments, where a small molecule compound is one that exists or is found in nature, that compound may be provided and/or utilized in accordance in the present disclosure in a form different from that in which it exists or is found in nature. Those of ordinary skill in the art will appreciate that, in some embodiments, a preparation of a particular small molecule compound that contains an absolute or relative amount of the compound, or of a particular form thereof, that is different from the absolute or relative (with respect to another component of the preparation including, for example, another form of the compound) amount of the compound or form that is present in a reference preparation of interest (e.g., in a primary sample from a source of interest such as a biological or environmental source) is distinct from the compound as it exists in the reference preparation or source.

Strong cation exchange resin: As used herein, the term “strong cation exchange resin” refers to an ion exchange resin characterized by one or more strong acid moieties, e.g., sulfonate and/or sulfonic acid moieties, covalently linked to a polymer support structure. In many embodiments, a strong cation exchange resin may be functionalized with one or more sulfonate and/or sulfonic acid moieties.

Strong anion exchange resin: As used herein, the term “strong anion exchange resin” refers to an ion exchange resin characterized by one or more tetrasubstituted ammonium cation moieties covalently linked to a polymer support structure. In many embodiments, a strong anion exchange resin may be functionalized with one or more tetrasubstituted ammonium moieties.

Type 1 resin: As used herein, a “type 1 resin” refers to a strong anion exchange resin characterized by one or more trimethylammonium cationic moieties covalently linked to a polymer matrix.

Type 2 resin: As used herein, a “type 2 resin” refers to a strong anion exchange resin characterized by one or more N-hydroxyethylene dimethylammonium cationic moieties covalently linked to a polymer matrix.

Weak anion exchange resin: As used herein, the term “weak anion exchange resin” refers to an ion exchange resin characterized by one or more amine and/or substituted amine (but not tetrasubstituted ammonium) moieties covalently linked to a polymer matrix. In some embodiments, a relevant amine comprises a neutral monosubstituted, disubstituted, or trisubstituted amine.

Weak cation exchange resin: As used herein, the term “weak cation exchange resin” refers to an ion exchange resin characterized by one or more weak acid moieties, e.g., carboxylate and/or carboxylic acid moieties, covalently linked to a polymer support structure. In many embodiments, a weak cation exchange resin is functionalized with one or more carboxylate and/or carboxylic acid moieties.

### DETAILED DESCRIPTION OF CERTAIN EMBODIMENTS

### Glycol Compounds

The present disclosure provides technologies relating to glycol compounds (e.g., to long chain glycol compounds, such as for example glycol compounds including about 5 to about 12 carbon atoms).

Glycol compounds are useful chemicals products with a wide array of applications in industrial and consumer goods. Among other things, for example, glycol compounds have been established to be particularly useful in production of polyurethanes, coatings, acrylates, adhesives, polyesters, resins, plasticizers, biodegradable plastics, pharmaceuticals, inks, and solvents.

Glycol compounds have been reported as being produced using reduction of dicarboxylic acids, e.g., adipic acid. Large scale (e.g., greater than about 100 grams) production of glycols typically proceeds through oxidation of petroleum distillates. For example, petroleum distillates comprising benzene may be hydrogenated to cyclohexane. Cyclohexane may then be oxidized to a mixture of cyclohexanone and cyclohexanol (i.e., KA oil). KA oil is then subject to ring-opening oxidation to provide adipic acid. Adipic acid may then be exhaustively reduced to provide 1,6-hexanediol (i.e., 1,6-hexane glycol). DOI:10.3389/fchem.2020.00185. Efforts to manufacture long chain glycol compounds (e.g., 1,6-hexanediol) have reported challenges, at least in part attributed to the comparatively reduced state of such long chain glycol compounds and the many chemical transformational steps after initial isolation of benzene from crude oil. DOI:10.15227/orgsyn.019.0048. In preparation of fossil glycol compounds, ion exchange is generally not performed. In a preparation of glycol by bio-process such as fermentation, ion exchange can be an important process, for example, in some embodiments, cultures contain inorganic ions and organic acids which are produced as metabolites.

The present disclosure provides improved technologies for production of certain glycol compounds (e.g., long chain glycol compounds, e.g., having 5-12 carbon atoms). In particular, the present disclosure provides technologies that achieve production of high purity aqueous preparations of such glycol compounds.

In some embodiments, a glycol compound has about 5 to about 12 carbon atoms, such as from 5 to 12 carbon atoms. In some embodiments, a glycol compound has about 5 to about 10 carbon atoms, such as from 5 to 10 carbon atoms. In some embodiments, a glycol compound has about 5 to about 8 carbon atoms, such as from 5 to 8 carbon atoms. In some embodiments, the glycol compound has about 6 carbon atoms, such as 6 carbon atoms.

In some embodiments, —OH group(s) of a glycol compound are at the terminus/i of the alkyl chain.

In some embodiments, a long chain glycol compound to which provided technologies are applicable may be or comprise one or more of 1,5-pentane glycol (1,5-pentanediol (PDO)), 1,6-hexane glycol (1,6-hexanediol (HDO)), 1,7-heptane glycol (1,7-heptanediol (HPDO)), 1,8-octane glycol (1,8-octanediol (ODO)), 1,9-nonane glycol (1,9-nonanediol (NDO)), 1,10-decane glycol (1,10-decanediol (DDO)), 1,11-undecane glycol (1,11-undecanediol), and 1,12-dodecane glycol (1,12-dodecanediol) and/or a regioisomer thereof.

In some embodiments, a glycol compound has the structure of HO-L-OH, wherein L is a bivalent C₂₋₁₅ (e.g., C₂₋₁₂, C₂₋₁₀, C₅₋₁₅, C₅₋₁₀, C₅₋₈, C₆₋₁₀, C₂, C₃, C₄, C₅, C₆, C₇, C₈, C₉, C₁₀, C₁₁, C₁₂, C₁₃, C₁₄ or C₁₅) aliphatic group. In some embodiments, L is linear. In some embodiments, L is branched. In some embodiments, L is saturated. In some embodiments, L is partially unsaturated. In some embodiments, L is a bivalent C₂₋₁₅ (e.g., C₂₋₁₂, C₂₋₁₀, C₅₋₁₅, C₅₋₁₀, C₅₋₈, C₆₋₁₀, C₂, C₃, C₄, C₅, C₆, C₇, C₈, C₉, C₁₀, C₁₁, C₁₂, C₁₃, C₁₄ or C₁₅) alkylene group. In some embodiments, L is bivalent linear C₅₋₁₀ alkylene. In some embodiments, L is —(CH₂)ₙ— wherein n is 2-15, 2-12, 5-15, 5-12, 6-15, 6-12, 6-10, 2, 3, 4, 5, 6, 7, 8, 9, or 10. In some embodiments, n is 5. In some embodiments, n is 6. In some embodiments, n is 7. In some embodiments, n is 8. In some embodiments, n is 9. In some embodiments, n is 10.

In some embodiments, a glycol compound is or comprises 1,5-pentanediol, 1,6-hexanediol, 1,7-hepanediol or 1,8-octanediol. In some embodiments, a glycol compound is or comprises 1,6-hexanediol. In some embodiments, a glycol compound is 1,6-hexanediol.

### Fermentation

Many important chemical compounds, including glycol compounds, have traditionally been produced from petroleum and/or by complicated chemical synthesis processes. Fermentative production offers a variety of advantages including, for example independence from the volatile global oil market and fewer transformative chemical conversions. Indeed, fermentation processes have been developed that can utilize renewable feedstocks (e.g., glucose or sucrose), and do not generate toxic byproducts. See, e.g., WO2015/042201 (Zymochem), WO2020/220001 (Zymochem); Jiang et al., Microbiol. Cell Factories 13:165, 2014; Beerthuis et al., Green Chem. 17:1341, 2015, etc. and references cited in the foregoing.

The present disclosure identifies the source of problem(s) associated with fermentative production (e.g., of glycol compounds as described herein), particularly when performed at large scale of total weight of glycol compound (e.g., above about 100 kg), or of large fermentation volume (e.g., 10-1000 kL as described herein), in that such production is necessarily in the context of an aqueous system (e.g., a cell, cell culture, or medium), which can complicate subsequent isolation of produced compounds. Indeed, fermentative production typically generates an aqueous system in which glycol compound(s) of interest is/are present in a complex mixture (e.g., with cell(s) and/or cell component(s)—e.g., products of cellular metabolism, etc).

The present disclosure appreciates that level of resin retention may present particular challenges when purification of large volume (e.g., in a range of about 10 kL to about 1000 kL), e.g., of a fermentation broth, is desired. In some embodiments, a volume is between about 10 kL and about 1000 kL. In some embodiments, a volume is between about 10 kL and about 800 kL. In some embodiments, a volume is between about 10 kL and about 600 kL. In some embodiments, a volume is between about 10 kL and about 400 kL. In some embodiments, a volume is between about 10 kL and about 200 kL. In some embodiments, a volume is between about 10 kL and about 100 kL. In some embodiments, a volume is between about 10 kL and about 80 kL. In some embodiments, a volume is between about 10 kL and about 70 kL. In some embodiments, a volume is between about 10 kL and about 60 kL. In some embodiments, a volume is between about 10 kL and about 50 kL. In some embodiments, a volume is between about 10 kL and about 40 kL. In some embodiments, a volume is between about 10 kL and about 30 kL. In some embodiments, a volume is between about 10 kL and about 20 kL. In some embodiments, a volume is between about 10 kL and about 15 kL. In some embodiments, a volume is between about 20 kL and about 800 kL. In some embodiments, a volume is between about 50 kL and about 800 kL. In some embodiments, a volume is between about 100 kL and about 800 kL. In some embodiments, a volume is between about 300 kL and about 800 kL. In some embodiments, a volume is between about 500 kL and about 800 kL. In some embodiments, a volume is between about 20 kL and about 100 kL. In some embodiments, a volume is between about 30 kL and about 80 kL. In some embodiments, a volume is between about 40 kL and about 70 kL. In some embodiments, a volume is about 10 kL, 20 kL, 30 kL, 40 kL, 50 kL, 60 kL, 70 kL, or 80 kL. In some embodiments, a volume is about 10 kL. In some embodiments, a volume is about 20 kL. In some embodiments, a volume is about 30 kL. In some embodiments, a volume is about 40 kL. In some embodiments, a volume is about 50 kL. In some embodiments, a volume is about 60 kL. In some embodiments, a volume is about 70 kL. In some embodiments, a volume is about 80 kL. In some embodiments, a volume is about 90 kL. In some embodiments, a volume is about 100 kL. In some embodiments, a volume is about 200 kL. In some embodiments, a volume is about 300 kL. In some embodiments, a volume is about 400 kL. In some embodiments, a volume is about 500 kL. In some embodiments, a volume is about 600 kL. In some embodiments, a volume is about 700 kL. In some embodiments, a volume is about 800 kL. In some embodiments, a volume is about 900 kL. In some embodiments, a volume is about 1000 kL.

### Aqueous Source and Initial Preparations

The present disclosure provides technologies for isolation and/or purification of glycol compound(s) from aqueous compositions (e.g., from aqueous sources, from compositions prepared therefrom, or from otherwise generated aqueous compositions).

In some embodiments, an aqueous composition (e.g., an aqueous source, an aqueous initial preparation or other aqueous preparation) from which glycol compound(s) may be isolated and/or purified is in accordance with the present disclosure comprising at least 50%, at least 55%, at least 60%, at least 65%, at least 75%, at least 80%, at least 85%, at least 90%, at least 95%, at least 97%, at least 98%, or at least 99% water (w/w) (e.g., as determined by Karl Fischer titration).

The present disclosure appreciates particular challenges associated with purifying glycol compounds from aqueous preparations (e.g., aqueous source preparations and/or aqueous “initial” preparations [i.e., relative to a purification step as described herein]), and furthermore appreciates that certain such challenges present particular difficulties at large scale (e.g., greater than 100 kg).

For example, the present disclosure appreciates that glycol compounds as described herein (e.g., long chain glycol compounds, for example having at least 5 carbon atoms, such as 5-12 carbon atoms) are often sufficiently hydrophilic that techniques such as extraction with organic solvent(s) cannot efficiently isolate them from an aqueous source or initial preparation. Accordingly, many typical such extraction technologies are not economical and/or not useful in this context. Furthermore, many organic solvents are environmentally undesirable; reliance on such agents to purify glycol compound(s) of interest risks undoing benefits achieved by switching from petroleum-based to fermentative production.

Furthermore, the present disclosure appreciates that, particularly where a source or initial aqueous preparation is a complex mixture (e.g., as is produced by fermentation), the presence of other materials in the preparation can further complicate purification of glycol compound(s) of interest (e.g., 1,5-pentane glycol (1,5-pentanediol (PDO)), 1,6-hexane glycol (1,6-hexanediol (HDO)), 1,7-heptane glycol (1,7-heptanediol (HPDO)), 1,8-octane glycol (1,8-octanediol (ODO)), 1,9-nonane glycol (1,9-nonanediol (NDO)), 1,10-decane glycol (1,10-decanediol (DDO)), 1,11-undecane glycol (1,11-undecanediol), and 1,12-dodecane glycol (1,12-dodecanediol) and/or a regioisomer thereof.). For example, extraction systems that fractionate glycol compound(s) into an aqueous fraction (e.g., rather than into an organic fraction) may also fractionate many other components of the source or initial aqueous preparation into that aqueous fraction, failing to achieve sufficient purification of the glycol compound(s). Furthermore, technologies such as distillation or evaporation, which primarily or solely remove volatile component(s), similarly may fail to achieve sufficient purification of glycol compound(s) of interest (e.g., 1,5-pentane glycol (1,5-pentanediol (PDO)), 1,6-hexane glycol (1,6-hexanediol (HDO)), 1,7-heptane glycol (1,7-heptanediol (HPDO)), 1,8-octane glycol (1,8-octanediol (ODO)), 1,9-nonane glycol (1,9-nonanediol (NDO)), 1,10-decane glycol (1,10-decanediol (DDO)), 1,11-undecane glycol (1,11-undecanediol), and 1,12-dodecane glycol (1,12-dodecanediol) and/or a regioisomer thereof.) from a complex aqueous source or initial preparation.

In some embodiments, an aqueous composition (e.g., an aqueous source, an aqueous initial preparation or other aqueous preparation) from which a glycol compound is isolated or purified in accordance with the present disclosure is or comprises a fermentation culture or broth. In some embodiments, an aqueous composition (e.g., an aqueous source or other aqueous preparation) from which a glycol compound is isolated or purified in accordance with the present disclosure comprises cell(s), such as microbial cells (e.g., bacterial cells). In some embodiments, a fermentation culture or broth may have an optical density within a range of about 10-200 at 600 nm as measure by spectrophotometry.

In some embodiments, an aqueous composition (e.g., an aqueous source, an aqueous initial preparation or other aqueous preparation) from which a glycol compound is isolated or purified in accordance with the present disclosure is or comprises cell component(s), e.g., microbial (e.g., bacterial) cell component(s) such as, for example, ions, lipids, metals, nucleic acids, polysaccharides, polypeptides, and/or combinations thereof. In some embodiments, a microbial cell (e.g., a bacterial cell) is or comprises a recombinant cell. In some embodiments, a microbial cell (e.g., a bacterial cell) is or comprises a cell that has been engineered to produce a glycol compound(s) of interest. In some embodiments, a microbial cell (e.g., a bacterial cell) has been engineered to produce a glycol compound that is a long chain glycol compound (e.g., having at least 5 carbon atoms, such as 5-12 carbon atoms). In some embodiments, a microbial cell (e.g., a bacterial cell) has been engineered to produce a glycol compound such as, e.g., 1,5-pentane glycol (1,5-pentanediol (PDO)), 1,6-hexane glycol (1,6-hexanediol (HDO)), 1,7-heptane glycol (1,7-heptanediol (HPDO)), 1,8-octane glycol (1,8-octanediol (ODO)), 1,9-nonane glycol (1,9-nonanediol (NDO)), 1,10-decane glycol (1,10-decanediol (DDO)), 1,11-undecane glycol (1,11-undecanediol), 1,12-dodecane glycol (1,12-dodecanediol), a regioisomer of any of the foregoing, and or combination(s) thereof.

In some embodiments, an aqueous composition (e.g., an aqueous source, an aqueous initial preparation or other aqueous preparation) from which a glycol compound is isolated or purified in accordance with the present disclosure includes a glycol compound of interest (e.g., a long chain glycol compound, such as a glycol compound comprising at least 5 carbon atoms, for example having about 5 to about 12 carbon atoms) at a concentration within a range of about 0.2 wt % to about 20 wt %. In some embodiments, a concentration is within a range of about 0.2 wt % to about 15 wt %. In some embodiments, a concentration is within a range of about 0.2 wt % to about 10 wt %. In some embodiments, a concentration is within a range of about 0.2 wt % to about 5 wt %. In some embodiments, a concentration is within a range of about 0.2 wt % to about 1 wt %. In some embodiments, a concentration is within a range of about 0.2 wt % to about 0.5 wt %. In some embodiments, a concentration is within a range of about 2 wt % to about 20 wt %. In some embodiments, a concentration is within a range of about 5 wt % to about 20 wt %. In some embodiments, a concentration is within a range of about 10 wt % to about 20 wt %. In some embodiments, a concentration is within a range of about 15 wt % to about 20 wt %. In some embodiments, a concentration is within a range of about 1 wt % to about 18 wt %. In some embodiments, a concentration is within a range of about 3 wt % to about 16 wt %. In some embodiments, a concentration is within a range of about 5 wt % to about 14 wt %. In some embodiments, a concentration is within a range of about 7 wt % to about 12 wt %. In some embodiments, a concentration is within a range of about 9 wt % to about 11 wt %. In some embodiments, a concentration is about 7 wt %, 8 wt %, 9 wt %, 10 wt %, 11 wt %, 12 wt %, or 13 wt %. In some embodiments, a concentration is about or at least about 0.2 wt %. In some embodiments, a concentration is about or at least about 0.5 wt %. In some embodiments, a concentration is about or at least about 1 wt %. In some embodiments, a concentration is about or at least about 2 wt %. In some embodiments, a concentration is about or at least about 3 wt %. In some embodiments, a concentration is about or at least about 4 wt %. In some embodiments, a concentration is about or at least about 5 wt %. In some embodiments, a concentration is about or at least about 6 wt %. In some embodiments, a concentration is about or at least about 7 wt %. In some embodiments, a concentration is about or at least about 8 wt %. In some embodiments, a concentration is about or at least about 9 wt %. In some embodiments, a concentration is about or at least about 10 wt %. In some embodiments, a concentration is about or at least about 11 wt %. In some embodiments, a concentration is about or at least about 12 wt %. In some embodiments, a concentration is about or at least about 13 wt %. In some embodiments, a concentration is about or at least about 14 wt %. In some embodiments, a concentration is about or at least about 15 wt %. In some embodiments, a concentration is about or at least about 16 wt %. In some embodiments, a concentration is about or at least about 17 wt %. In some embodiments, a concentration is about or at least about 18 wt %. In some embodiments, a concentration is about or at least about 19 wt %. In some embodiments, a concentration is about or at least about 20 wt %.

In some embodiments, an aqueous preparation before ion exchange, e.g., an aqueous initial preparation contains high levels of ions, e.g., inorganic ions (e.g., sulfate, phosphate, pyruvate, lithium, sodium, potassium, ammonium, calcium, magnesium, iron(II), iron(III), aluminum, chloride), organic ions (e.g., acetate, lactate, malate, succinate, maleate), amino acids (e.g., valine, and/or leucine) and/or impurities. In some embodiments, levels of one or more ions and/or impurities are independently about or at least about 50, 60, 70, 80, 90, 100, 150, 200, 300, 400, 500, 600, 700, 800, 900, 1000, 1500, 2000, 3000, 4000, 5000, 6000, 7000, 8000, or 10000 ppm (w/w). For example, in some embodiments, level of K⁺ is about or at least about 3000 ppm (e.g., in some embodiments, about 3100 ppm). In some embodiments, level of Mg²⁺ is about or at least about 90 ppm. In some embodiments, level of pyruvate is about or at least 3000 ppm (e.g., in some embodiments, about 3200 ppm). In some embodiments, level of SO₄²⁻ is about or at least about 350 ppm. In some embodiments, level of PO₄³⁻ is about or at least about 2000 ppm (e.g., in some embodiments, about 2400 ppm). As those skilled in the art appreciate, phosphate may exist in various forms such as H₂PO₄⁻, HPO₄²⁻, and PO₄³⁻. A level/concentration of phosphate or PO₄³⁻ described in the present disclosure may be the level/concentration of all these form combined.

In some embodiments, an aqueous source composition, e.g., an aqueous initial preparation, is or comprises a fermentation culture. In some embodiments, an aqueous source composition has been derived from an aqueous source composition (such as a fermentation culture), for example by one or more steps of concentration, separation (e.g., filtration), etc. In some embodiments, an aqueous source composition has been or becomes (prior to or after performance of one or more steps—such as IEX and/or filtration—as specifically provided herein) subjected to one or more technologies for separation or purification of components include those based on, for example, physical size or solubility and/or chemical properties (e.g., charge or other types of affinity). For example, filtration technologies can separate by size and/or solubility. Other technologies (e.g., column chromatography) can be used to separate based on charge or other chemical properties.

In some embodiments, solid matters and/or microorganism are removed through filtration and/or centrifugation. Various such technologies may be utilized in accordance with the present disclosure. In some embodiments, a filtration is or comprises membrane separation. In some embodiments, for a filtration pore size is about 10 μm or less. In some embodiments, centrifugation is or comprises continuous centrifugation. Examples of continuous centrifuge include cage centrifuge, disc centrifuge and nozzle centrifuge. In some embodiments, a technology, e.g., a centrifugation technology, is utilized in combination with another technology, e.g., another centrifugation technology.

In some embodiments, a membrane is utilized for separation. In some embodiments, for a filtration pore size is about 10 μm or less. In some embodiments, a membrane may be utilized alone or two or more membranes of different types, pore sizes, shapes, etc. may be utilized. In some embodiments, a membrane filtration is performed with a particular pore size, followed by a filtration with a pore size of a smaller diameter. In some embodiments, a membrane filtration is performed with a particular pore size, followed by two or more filtrations with smaller pore sizes and/or different membrane shapes.

In some embodiments, filtration membranes may confer additional advantages when alternative shapes and/or topologic convolutions are employed. In some embodiments, a membrane is a flat membrane. In some embodiments, a membrane is a hollow fiber. In some embodiments, a membrane is spiral. In some embodiments, a membrane is tubular. In some embodiments, a membrane is pleated. In some embodiments, a membrane is or comprises ceramic membrane.

Many filtration technologies may be utilized in accordance with the present disclosure, e.g., dead-end method, tangential flow method, etc.

In some embodiments, cells, microorganisms, etc., are deactivated or killed at the end of fermentation (biomass deactivation). In some embodiments, such deactivation and/or killing can reduce or prevent their prolonged activity after fermentation and/or contamination during, e.g., long storage, downstream operations, etc. Various technologies can be utilized for deactivation/killing in accordance with the present disclosure. For example, in some embodiments, cells, microorganisms, etc., are subjected to high temperature, e.g., short term high temperature similar to pasteurization via heat exchanger. In some embodiments, biomass deactivation is performed at a temperature about 50 to about 80° C., for a duration of about 1 to about 1o minutes. In some embodiments, cell lysis is reduced or prevented by flocculation or agglomeration, which can also promote better separation during cell separation. In some embodiments, a method comprises biomass deactivation as described herein. In some embodiments, a method does not utilize biomass deactivation as described herein.

In some embodiments, an aqueous composition (e.g., an aqueous source, an aqueous initial preparation or other aqueous preparation) from which a glycol compound is isolated or purified in accordance with the present disclosure has a volume of between about 1 kL and about 1000 kL. In some embodiments, a volume is between about 10 kL and about 1000 kL. In some embodiments, a volume is between about 10 kL and about 800 kL. In some embodiments, a volume is between about 10 kL and about 600 kL. In some embodiments, a volume is between about 10 kL and about 400 kL. In some embodiments, a volume is between about 10 kL and about 200 kL. In some embodiments, a volume is between about 10 kL and about 100 kL. In some embodiments, a volume is between about 10 kL and about 80 kL. In some embodiments, a volume is between about 10 kL and about 70 kL. In some embodiments, a volume is between about 10 kL and about 60 kL. In some embodiments, a volume is between about 10 kL and about 50 kL. In some embodiments, a volume is between about 10 kL and about 40 kL. In some embodiments, a volume is between about 10 kL and about 30 kL. In some embodiments, a volume is between about 10 kL and about 20 kL. In some embodiments, a volume is between about 10 kL and about 15 kL. In some embodiments, a volume is between about 20 kL and about 800 kL. In some embodiments, a volume is between about 50 kL and about 800 kL. In some embodiments, a volume is between about 100 kL and about 800 kL. In some embodiments, a volume is between about 300 kL and about 800 kL. In some embodiments, a volume is between about 500 kL and about 800 kL. In some embodiments, a volume is between about 20 kL and about 100 kL. In some embodiments, a volume is between about 30 kL and about 80 kL. In some embodiments, a volume is between about 40 kL and about 70 kL. In some embodiments, a volume is about 10 kL, 20 kL, 30 kL, 40 kL, 50 kL, 60 kL, 70 kL, or 80 kL. In some embodiments, a volume is about or at least about 10 kL. In some embodiments, a volume is about or at least about 20 kL. In some embodiments, a volume is about or at least about 30 kL. In some embodiments, a volume is about or at least about 40 kL. In some embodiments, a volume is about or at least about 50 kL. In some embodiments, a volume is about or at least about 60 kL. In some embodiments, a volume is about or at least about 70 kL. In some embodiments, a volume is about or at least about 80 kL. In some embodiments, a volume is about or at least about 90 kL. In some embodiments, a volume is about or at least about 100 kL. In some embodiments, a volume is about or at least about 200 kL. In some embodiments, a volume is about or at least about 300 kL. In some embodiments, a volume is about or at least about 400 kL. In some embodiments, a volume is about or at least about 500 kL. In some embodiments, a volume is about or at least about 600 kL. In some embodiments, a volume is about or at least about 700 kL. In some embodiments, a volume is about or at least about 800 kL. In some embodiments, a volume is about or at least about 900 kL. In some embodiments, a volume is about or at least about 1000 kL.

The present disclosure provides a variety of technologies useful in the purification of glycol compounds e.g., from aqueous preparations that may contain other ionizable molecules and/or insoluble matter. Particular provided technologies relate to separation of glycol compound(s) as described herein (e.g., 1,5-pentane glycol (1,5-pentanediol (PDO)), 1,6-hexane glycol (1,6-hexanediol (HDO)), 1,7-heptane glycol (1,7-heptanediol (HPDO)), 1,8-octane glycol (1,8-octanediol (ODO)), 1,9-nonane glycol (1,9-nonanediol (NDO)), 1,10-decane glycol (1,10-decanediol (DDO)), 1,11-undecane glycol (1,11-undecanediol), and 1,12-dodecane glycol (1,12-dodecanediol) and/or a regioisomer thereof) from aqueous preparations (e.g., from aqueous source preparations).

In some embodiments, an aqueous initial preparation in accordance with the present disclosure may be or comprise a cell (e.g., a microbial cell) or culture thereof, or medium therefrom, that comprises a glycol compound(s) as described herein (e.g, 1,5-pentane glycol (1,5-pentanediol (PDO)), 1,6-hexane glycol (1,6-hexanediol (HDO)), 1,7-heptane glycol (1,7-heptanediol (HPDO)), 1,8-octane glycol (1,8-octanediol (ODO)), 1,9-nonane glycol (1,9-nonanediol (NDO)), 1,10-decane glycol (1,10-decanediol (DDO)), 1,11-undecane glycol (1,11-undecanediol), and 1,12-dodecane glycol (1,12-dodecanediol) and/or a regioisomer thereof), e.g., that may have undergone fermentation; in some embodiments, an aqueous initial preparation is a preparation that has been prepared from a fermented composition. In some embodiments, an aqueous initial preparation includes one or more cells or cell components. In some embodiments, an aqueous initial preparation does not include cellular material (e.g., was prepared from a non-cellular source and/or otherwise was rendered substantially free of cell component(s)).

Those skilled in the art, reading the present disclosure, will appreciate that many of its teachings are applicable to aqueous preparations (e.g., aqueous initial preparations) regardless of the original source from which they may have been obtained or how they may otherwise have been prepared.

In some embodiments, an aqueous initial preparation includes phosphate. In some embodiments, an aqueous initial preparation includes sulfate. In some embodiments, an aqueous initial preparation includes pyruvate. In some embodiments, an aqueous initial preparation includes a glycol compound. In some embodiments, an aqueous initial preparation includes 1,6-hexane glycol (1,6-hexanediol). In some embodiments, an aqueous initial preparation includes DNA and DNA fragments. In some embodiments, an aqueous initial preparation includes RNA and RNA fragments. In some embodiments, an aqueous initial preparation includes glucose. In some embodiments, an aqueous initial preparation includes palmitate and palmitic acid. In some embodiments, an aqueous initial preparation includes oleate and oleic acid. In some embodiments, an aqueous initial preparation includes glycerin.

In some embodiments, an aqueous initial preparation is substantially free of insoluble microbial cell matter. In some embodiments, an aqueous initial preparation is substantially free of insoluble bacterial cell matter. In some embodiments, an aqueous initial preparation is substantially free of glycerol. In some embodiments, an aqueous initial preparation is substantially free of cellulose. In some embodiments, an aqueous initial preparation is substantially free of peptidoglycan. In some embodiments, an aqueous initial preparation is substantially free of teichoic acids. In some embodiments, an aqueous initial preparation is substantially free of protein and protein fragments. In some embodiments, an aqueous initial preparation is substantially free of lipopolysaccharides. In some embodiments, an aqueous initial preparation is substantially free of molecules above 5 kD. In some embodiments, an aqueous initial preparation is substantially free of molecules above 200 D. In some embodiments, an aqueous preparation is substantially free of cells (e.g., bacterial cells), proteins, polysaccharides, multivalent ions, and/or compounds with an molecular weight of about 200, 500, 1000, 2000 or 5000. In some embodiments, provided technologies comprise microfiltration, which can remove or reduce levels of, e.g., bacterial cells, proteins, polysaccharide and/or compounds with molecular weights species 5000. In some embodiments, provided technologies comprise nanofiltration, which can remove or reduce levels of, e.g., peptides, multivalent ions, and/or compounds with molecular weights species 5000.

In some embodiments, an aqueous preparation (e.g., an aqueous initial preparation) comprises hydrochloric acid. In some embodiments, an aqueous preparation (e.g., an aqueous initial preparation) comprises sodium hydroxide. In some embodiments, an aqueous preparation (e.g., an aqueous initial preparation) comprises a mixture of glycol compounds.

In some embodiments, an aqueous preparation (e.g., an aqueous initial preparation) to which provided technologies, and in particular to which a provided IEX process, is applied has been pH adjusted, for example, to amplify or attenuate ionic strength of an aqueous preparation so as to preferentially target ions of interest.

In some embodiments, an aqueous preparation (e.g., an aqueous initial preparation) to which provided technologies, and in particular to which a provided IEX process, is applied (i) includes a plurality of distinct chemical entities (e.g., including a glycol compound(s) of interest and one or more other entity(ies) from which such glycol compound(s) is/are to be separated) that are not readily discriminated from one another based on size exclusion/separation (e.g., by filtration) and/or (ii) has been exposed to one or more size exclusion/separation (e.g., filtration) treatments.

Among other things, the present disclosure provides particularly useful technologies for processing certain aqueous preparations (e.g., fermentation cultures or preparations generated therefrom), in particular by filtration. For example, the present disclosure demonstrates particular utility of certain microfiltration processes.

In some embodiments, an aqueous preparation (e.g., an aqueous initial preparation) to which provided technologies, and in particular to which a provided IEX process is applied is characterized by reduced optical density as compared with that of a fermentation culture (e.g., from which the aqueous preparation) has been prepared. In some embodiments, an aqueous preparation (e.g., an aqueous initial preparation) to which provided technologies, and in particular to which a provided IEX process is applied is characterized by electrical conductance (e.g., as measured by μS/cm) within a range of about 10-5000 (e.g., about 10, 20, 50, 100, 200, 500, 1000, 2000, 3000, 4000, 5000, etc.) μS/cm. In some embodiments, an aqueous initial preparation has an electrical conductance between about 10 μS/cm and about 5000 μS/cm. In some embodiments, an aqueous initial preparation has an electrical conductance between about 10 μS/cm and about 4000 μS/cm. In some embodiments, an aqueous initial preparation has an electrical conductance between about 10 μS/cm and about 3000 μS/cm. In some embodiments, an aqueous initial preparation has an electrical conductance between about 10 μS/cm and about 2000 μS/cm. In some embodiments, an aqueous initial preparation has an electrical conductance between about 10 μS/cm and about 1000 μS/cm. In some embodiments, an aqueous initial preparation has an electrical conductance between about 10 μS/cm and about 500 μS/cm. In some embodiments, an aqueous initial preparation has an electrical conductance between about 10 μS/cm and about 100 μS/cm. In some embodiments, an aqueous initial preparation has an electrical conductance between about 50 μS/cm and about 5000 μS/cm. In some embodiments, an aqueous initial preparation has an electrical conductance between about 75 μS/cm and about 5000 μS/cm. In some embodiments, an aqueous initial preparation has an electrical conductance between about 100 μS/cm and about 5000 μS/cm. In some embodiments, an aqueous initial preparation has an electrical conductance between about 50 μS/cm and about 1000 μS/cm. In some embodiments, an aqueous initial preparation has an electrical conductance between about 75 μS/cm and about 500 μS/cm. In some embodiments, an aqueous initial preparation has an electrical conductance between about 90 μS/cm and about 110 μS/cm. In some embodiments, an aqueous initial preparation has an electrical conductance of about 70 μS/cm, 85 μS/cm, 100 μS/cm, 115 μS/cm, or 130 μS/cm. In some embodiments, an aqueous initial preparation has an electrical conductance of about 70 μS/cm. In some embodiments, an aqueous initial preparation has an electrical conductance of about 80 μS/cm. In some embodiments, an aqueous initial preparation has an electrical conductance of about 90 μS/cm. In some embodiments, an aqueous initial preparation has an electrical conductance of about 100 μS/cm. In some embodiments, an aqueous initial preparation has an electrical conductance of about 110 μS/cm. In some embodiments, an aqueous initial preparation has an electrical conductance of about 120 μS/cm. In some embodiments, an aqueous initial preparation has an electrical conductance of about 130 μS/cm. In some embodiments, an aqueous initial preparation has an electrical conductance of about 150 μS/cm. In some embodiments, an aqueous initial preparation has an electrical conductance of about 200 μS/cm.

### IEX

Among other things, the present disclosure provides ion exchange chromatography (IEX) technologies that are useful for the isolation and/or purification of glycol compound(s) (e.g., long chain glycol compounds such as those having at least 5, such as 5-12, carbon atoms). Those skilled in the art appreciate that glycol compounds include alcohol moieties, are typically soluble in water, and can be difficult to differentiate (e.g., by chromatographic means) from other polar molecules in an aqueous preparation, particularly if/when they are in intimate admixture with one another. Selective isolation of an alcoholic functionality from an aqueous preparation, particularly one that comprises one or more other cations and/or anions can present difficult technical challenge(s).

The present disclosure provides technologies that overcome these challenges.

Among other things, the present disclosure provides insights that:
(i) particularly good results (e.g., less resin retention) can be achieved for purification of a glycol compound as described herein (e.g., a long chain glycol compound, such as one having at least 5 carbon atoms e.g., in some embodiments 5-12 carbon atoms), and in particular for purification of such compound from an aqueous source (e.g., a fermentation culture or composition obtained therefrom) through use of a hydrophilic resin (e.g., as compared with results that might be achieved with a typical hydrophobic resin such as a polystyrene support);
(ii) exposing a chromatography resin (e.g., an ion exchange resin) to a glycol compound prior to utilizing the resin to isolate that compound from a sample (e.g., from an aqueous sample—e.g., an aqueous initial preparation as described herein) improves purification efficiency achieved by the resin. That is, more glycol compound passes through a resin that has been pre-treated as described herein than does through an otherwise comparable resin that has not been so pre-treated;
(iii) exposing a chromatography resin on which a glycol compound as described herein (e.g., a long chain glycol compound, such as one having at least 5 carbon atoms e.g., in some embodiments 5-12 carbon atom) has been retained to an aqueous solution comprising a glycol compound (e.g., a pre-IEX composition or an aqueous sample—e.g., an aqueous initial preparation as described herein) can usefully remove non-glycol compound material(s) from the solution to a greater extent than it removes the glycol compound; and
(iv) exposing a chromatography resin on which a glycol compound as described herein (e.g., a long chain glycol compound, such as one having at least 5 carbon atoms e.g., in some embodiments 5-12 carbon atom) has been retained to wash with a suitable solvent system, e.g., water, an alcohol (e.g., a short chain alcohol such as methanol and ethanol), an organic solvent (e.g., THF and acetone) and a mixture of two or more of such solvents, can usefully recover the glycol compound.

### Resin

In some embodiments, the present disclosure provides a surprising teaching that hydrophilic ion exchange resins can be particularly effective in the purification of glycol compounds (e.g., a long chain glycol compound, such as one having at least 5 carbon atoms e.g., in some embodiments 5-12 carbon atom) from aqueous preparations.

In some embodiments, an IEX resin utilized in accordance with the present disclosure is hydrophilic.

In some embodiments, an IEX resin utilized in accordance with the present disclosure is lipophobic.

In some embodiments, an IEX resin utilized in accordance with the present disclosure is lipophilic.

In some embodiments, an IEX resin utilized in accordance with the present disclosure is hydrophobic.

In some embodiments, an IEX resin utilized in accordance with the present disclosure is or comprises a polymer (e.g., a functionalized polymer). In some embodiments, an IEX resin is or comprises a polystyrene, a polyacrylate, or a polymethacrylate. In some embodiments, an IEX resin comprises a base polymer functionalized with acidic or basic groups. In some embodiments, an IEX base polymer is polystyrene, polyacrylate, or polymethacrylate. In some embodiments, an IEX base polymer comprises crosslinking and/or copolymerization, e.g., with di-functional monomer, e.g., divinylbenzene (DVB). In some embodiments, a base polymer is functionalized by incorporation of acidic groups, e.g., sulfonate groups. In some embodiments, a base polymer is functionalized by incorporation of amine groups. In some embodiments, a base polymer is functionalized by incorporation of quaternary ammonium groups. In some embodiments, an IEX resin is or comprises a cation exchange resin. In some embodiments, a cation exchange resin is or comprises a styrene-DVB (divinylbenzene) resin bearing acidic groups. In some embodiments, a cation exchange resin is or comprises a styrene-DVB (divinylbenzene) resin bearing sulfonate groups. In some embodiments, an IEX resin is or comprises an anion exchange resin. In some embodiments, an anion exchange resin is or comprises an arylic-DVB resin. In some embodiments, an anion exchange resin is or comprises an arylic-DVB resin bearing quaternary ammonium groups.

In some embodiments, an IEX resin is or comprises a polystyrene support structure.

In some embodiments, an ion exchange resin is or comprises a polyacrylate support structure.

In some embodiments, an ion exchange resin is or comprises a polymethacrylate support structure.

In some embodiments, an ion exchange resin is a cross-linked resin. In some embodiments, the degree of cross-linking is within a range between about 1% and about 40%. In some embodiments, the degree of cross-linking is within a range between about 1% and about 35%. In some embodiments, the degree of cross-linking is within a range between about 1% and about 30%. In some embodiments, the degree of cross-linking is within a range between about 1% and about 25%. In some embodiments, the degree of cross-linking is within a range between about 1% and about 20%. In some embodiments, the degree of cross-linking is within a range between about 2% and about 20%. In some embodiments, the degree of cross-linking is within a range between about 3% and about 20%. In some embodiments, the degree of cross-linking is within a range between about 4% and about 20%. In some embodiments, the degree of cross-linking is within a range between about 4% and about 15%. In some embodiments, the degree of cross-linking is about 1%, 2%, 3%, 4%, 5%, 6%, 7%, 8%, 9%, 10%, 11%, 12%, 13%, 14%, 15%, 16%, 17%, 18%, 19%, or 20%. In some embodiments, the degree of cross-linking is about 1%. In some embodiments, the degree of cross-linking is about 2%. In some embodiments, the degree of cross-linking is about 4%. In some embodiments, the degree of cross-linking is about 5%. In some embodiments, the degree of cross-linking is about 10%. In some embodiments, the degree of cross-linking is about 15%. In some embodiments, the degree of cross-linking is about 20%.

In some embodiments, an ion exchange resin is or comprises a polystyrene resin. In some embodiments, an ion exchange resin is or comprises a polyacrylic resin. In some embodiments, an ion exchange resin is or comprises a hydrogel system. In some embodiments, an ion exchange resin is or comprises a gel type, a porous type and/or a high-porous type. In some embodiments, an ion exchange resin is or comprises a gel. In some embodiments, an ion exchange resin is or comprises a powder. In some embodiments, an ion exchange resin is or comprises spheres. In some embodiments, an ion exchange resin is or comprises fibers. In some embodiments, an ion exchange resin is or comprises a membrane. In some embodiments, an ion exchange resin is or comprises a microporous material. In some embodiments, an ion exchange resin is or comprises a macroporous material. In some embodiments, crosslinking (e.g., degree and/or type of crosslinking) determines one or more material properties (e.g., gel nature, degree of porosity, pore size, etc) of a support matrix.

In some embodiments, a resin is functionalized by various groups as described herein. In some embodiments, average functional group density of a resin is about 0.1 to 4.0 (e.g., about 0.1-3.5, 0.1-3.0, 0.1-2.0, or 0.5-3.0) eq/L. In some embodiments, it is about 0.1 eq/L. In some embodiments, it is about 0.2 eq/L. In some embodiments, it is about 0.3 eq/L. In some embodiments, it is about 0.5 eq/L. In some embodiments, it is about 1.0 eq/L. In some embodiments, it is about 1.5 eq/L. In some embodiments, it is about 2.0 eq/L. In some embodiments, it is about 2.5 eq/L. In some embodiments, it is about 3.0 eq/L. In some embodiments, it is about 3.5 eq/L. In some embodiments, it is about 4.0 eq/L.

### Particle Size

In some embodiments, an ion exchange resin utilized in accordance with the present disclosure is characterized by particle size.

In some embodiments, the average particle size is between about 100 μm and about 1500 μm. In some embodiments, the average particle size is between about 100 μm and about 1100 μm. In some embodiments, the average particle size is between about 100 μm and about 800 μm. In some embodiments, the average particle size is between about 100 μm and about 500 μm. In some embodiments, the average particle size is between about 500 μm and about 1500 μm. In some embodiments, the average particle size is between about 1000 μm and about 1500 μm. In some embodiments, the average particle size is between about 200 μm and about 1300 μm. In some embodiments, the average particle size is between about 300 μm and about 1100 μm. In some embodiments, the average particle size is between about 400 μm and about 1000 μm. In some embodiments, the average particle size is between about 500 μm and about 900 μm. In some embodiments, the average particle size is between about 600 μm and about 800 μm. In some embodiments, the average particle size is between about 100, 200, 300, 400, 500, 600, 700, 800, 900, 1000, 1100, 1200, 1300, 1400, or 1500 μm.

### Porosity

In some embodiments, an ion exchange resin utilized in accordance with the present disclosure is characterized by its porosity.

Resin of various porosity size may be utilized in accordance with the present disclosure. In some embodiments, the porosity is between about 1 nm and about 250 nm. In some embodiments, the porosity is between about 1 nm and about 175 nm. In some embodiments, the porosity is between about 1 nm and about 150 nm. In some embodiments, the porosity is between about 1 nm and about 125 nm. In some embodiments, the porosity is between about 1 nm and about 100 nm. In some embodiments, the porosity is between about 1 nm and about 75 nm. In some embodiments, the porosity is between about 1 nm and about 60 nm. In some embodiments, the porosity is between about 1 nm and about 50 nm.

### Functional Groups

In some embodiments, an ion exchange resin comprises functional group(s). In some embodiments, a utilized ion exchange resin comprises functional group(s) compatible with/particularly suitable for purification of, a particular glycol compound(s) of interest.

In some embodiments, an ion exchange resin is or comprises a cation exchange resin. In some embodiments, an ion exchange resin is or comprises a strong cation exchange resin. In some embodiments, a strong cation exchange resin is or comprises strong acid moieties covalently linked to a polymer support structure. In some embodiments, a strong cation exchange resin is or comprises one or more sulfonate and/or sulfonic acid moieties covalently linked to a polymer support structure. In some embodiments, a strong cation exchange resin is commercially available, e.g., SK1BH, DIAION, Mitsubishi, Tokyo, Japan.

In some embodiments, an ion exchange resin is or comprises a weak cation exchange resin. In some embodiments, a weak cation exchange resin is or comprises one or more carboxylate and/or carboxylic acid moieties covalently linked to a polymer support structure. In some embodiments, a strong cation exchange resin is commercially available, e.g., WK60L, WK10, etc.

In some embodiments, an ion exchange resin is or comprises an anion exchange resin. In some embodiments, an ion exchange resin is or comprises a weak anion exchange resin. In some embodiments, a weak anion exchange resin is or comprises one or more amine and/or substituted amine moieties (but not tetra-substituted ammonium moieties) covalently linked to a polymer support structure; in some such embodiments, an amine comprises a neutral monosubstituted, disubstituted, or trisubstituted amine. In some embodiments, a weak anion exchange resin is commercially available, e.g., WA21J, IRA67, etc.

In some embodiments, an ion exchange resin is or comprises a strong anion exchange resin. In some embodiments, a strong anion exchange resin is or comprises one or more tetrasubstituted ammonium cationic moieties covalently linked to a polymer support structure. In many embodiments, a strong anion exchange resin is functionalized with one or more tetrasubstituted ammonium moieties. In some embodiments, a strong anion exchange resin is commercially available, e.g., SA10AOH, HPR4580 Cl, SCAV4 Cl, etc.

### Anion Exchange Resins—Type 1

In some embodiments, a strong anion exchange resin is or comprises one or more trimethylammonium cationic moieties covalently linked to a polymer support structure.

HPR4580 Cl (SAER): a commercial example of a type 1 strong anion exchange resin comprising a gel polyacrylate. DuPont, Wilmington DE. Physicochemical properties: (a) polyacrylic gel; (b) strong anion exchange resin; (c) type 1; (d) molar equivalents: ≥1.25 eq/L (Cl⁻ form); (e) water retention capacity: 58.0%-62.0% (Cl⁻ form); (f) particle diameter: 700 μm-950 μm; (g) swelling (Cl⁻-->OH⁻)≤25%.

SCAV4 Cl (SAER): a commercial example of a type 1 strong anion exchange resin comprising a macroporous polyacrylate. DuPont, Wilmington DE. Physicochemical properties: (a) polyacrylic macroporous; (b) strong anion exchange resin; (c) type 1; (d) molar equivalents: ≥0.80 eq/L (Cl- form); (e) water retention capacity: 66.0%-72.0% (Cl⁻ form); (f) particle diameter: 630 μm-850 μm.

### Anion Exchange Resins—Type 2

In some embodiments, a strong anion exchange resin is or comprises one or more dimethylethanol ammonium cationic moieties covalently linked to a polymer support structure.

### Pre-Treatment

In some embodiments, an IEX resin may be subjected to one or more pre-treatment steps before an aqueous initial preparation as described herein (e.g., comprising a glycol compound of interest, which may be the glycol compound utilized in the pre-treatment), is contacted with the resin.

In some embodiments, a pre-treatment comprises exposure of an IEX resin to one or more of water (e.g., deionized water; e.g., for H-type resin), a hydroxide (e.g., an aqueous hydroxide solution; e.g., LiOH (e.g., aqueous LiOH), NaOH (e.g., aqueous NaOH), KOH (e.g., aqueous KOH)), a strong acid (e.g., an aqueous acid solution), etc. according to resin types.

Alternatively or additionally, in some embodiments, a pre-treatment comprises one or more pre-masking steps in which an IEX resin is pre-treated with a glycol compound before an aqueous initial preparation as described herein (e.g., comprising a glycol compound of interest, which may be the glycol compound utilized in the pre-treatment), is contacted with the resin. For pre-masking, a glycol compound may be provided as a solution. Typically, such a solution comprises no or low levels of impurities, e.g., ions, compared to a composition from which a glycol compound is purified from (e.g., an initial preparation). For example, in some embodiments, each ion or impurity independently has a concentration of about or no more than about 50, 40, 30, 20, 10, 9, 8, 7, 6, 5, 4, 3, 2, 1, 0.5, 0.2 or 0.1 ppm (w/w). In some embodiments, a glycol compound is provided as a water solution. In some embodiments, a glycol compound is provided as a solution, e.g., water solution, of a pure compound. In some embodiments, concentration of a solution, e.g., a pre-masking solution, is no more than about 50%, e.g., about 0.1%-50%, or about 0.1%, 0.2%, 0.5%, 1%, 2%, 5%, 10%, 15%, 20%, 25%, 30%, 40% or 50% (wt %) of a glycol compound.

In some embodiments, a glycol compound is applied at about or at least about 1-1000, e.g., about 1-500, 1-200, 1-100, 1-50, 1-20, 1-10, or about 1, 2, 5, 10, 15, 20, 25, 30, 40, 50, 60, 70, 80, 90, 100, 150, 200, 300, 400, 500, 600, 700, 800, 900 or 1000 mg/mL resin. In some embodiments, it is about 1 mg/mL resin. In some embodiments, it is about 2 mg/mL resin. In some embodiments, it is about 5 mg/mL resin. In some embodiments, it is about 10 mg/mL resin. In some embodiments, it is about 20 mg/mL resin. In some embodiments, it is about 50 mg/mL resin. In some embodiments, it is about 100 mg/mL resin. In some embodiments, it is about 150 mg/mL resin. In some embodiments, it is about 200 mg/mL resin. In some embodiments, it is about 500 mg/mL resin. In some embodiments, it is about 1000 mg/mL resin.

In some embodiments, an aqueous initial preparation has been pH-adjusted or otherwise treated so as to enhance retention of the glycol compound of interest, relative to at least one other component of the aqueous initial composition, to the IEX resin.

In some embodiments, an aqueous initial composition may have been treated to achieve protonation of a functional group (e.g., by a strong proton donor, such as a strong acid), e.g., to form a cation. Without wishing to be bound by any particular theory, it is proposed that such a charged cation in an aqueous initial preparation may show preferential electrostatic interactions proximal to an ion exchange resin surface.

Alternatively or additionally, in some embodiments, an aqueous initial preparation may have been pH adjusted (or otherwise treated) so that a functional group is deprotonated, e.g., to form an anion. Without wishing to be bound by any particular theory, it is proposed that such a charged anion in an aqueous initial preparation may show electrostatic interactions proximal to an ion exchange resin surface.

In some embodiments of the present disclosure, an IEX resin is pre-treated (e.g., pre-masked) with a protonated or deprotonated form of a compound of interest; in some such embodiments, the IEX resin is then contacted with an aqueous initial preparation containing the protonated or deprotonated form.

In some embodiments, a pre-masking step involves contacting an IEX resin with an aqueous solution comprising a glycol compound in a range of about 0.1% to about 50% wt/wt; in some embodiments, such range is between about 0.1% to about 2%. In some embodiments, an IEX resin is contacted with an aqueous solution comprising a relevant glycol compound at about 0.5% wt/wt. In some embodiments, an IEX resin is contacted with an aqueous solution comprising a relevant glycol compound at about 0.1% wt/wt.

In some embodiments, pre-masking involves contacting for a period of time that is about 20 mins, about 30 mins, about 40 mins, about 50 mins, about 60 mins, about 70 mins, about 80 mins, about 90 mins or more, optionally under mixing (e.g., stirring) conditions. In some embodiments, such time period is about 60 mins.

In some embodiments, pre-masking significantly reduces loss of a glycol compound. In some embodiments, it reduces the loss by about or at least about 1.5, 2, 3, 4, 5, 6, 7, 8, 9, or 10 fold (loss without post-recovering/loss with post-recovering). In some embodiments, a reduction is about or at least about 1.5 fold. In some embodiments, it is about or at least about 2 fold. In some embodiments, it is about or at least about 5 fold. In some embodiments, it is about or at least about 10 fold.

In some embodiments, pre-treatment is conducted in a batch, columnar, or continuous flow method. In some embodiments, pre-treatment is conducted in a batch method. In some embodiments, pre-treatment is conducted in columnar method. In some embodiments, pre-treatment is conducted in a continuous flow method.

### Purification

In accordance with the present disclosure, a preparation, e.g., an aqueous initial preparation, is purified by contact with an IEX resin (e.g., by IEX chromatography) as described herein (e.g., by contact with a resin that has been pre-treated—e.g., pre-masked as described herein) and/or that is washed as described herein. Alternatively or additionally, in some embodiments, an aqueous initial preparation is purified by contact with an IEX resin that is a hydrophilic resin. In some embodiments, an aqueous initial preparation is purified by contact with an IEX resin that is a hydrophobic resin. In some embodiments, a hydrophilic resin provides low level of loss of a purification target, e.g., a glycol compound (e.g., a glycol compound including about 5 to about 12 carbon atoms such as HDO), than a hydrophobic resin. In some embodiments, pre-masking reduces loss of a purification target, e.g., a glycol compound (e.g., a glycol compound including about 5 to about 12 carbon atoms such as HDO).

In some embodiments, a preparation, e.g., an aqueous initial preparation, is purified by contact with a cation exchange resin. In some embodiments, it is contacted with an anion exchange resin. In some embodiments, it is contacted with both a cation exchange resin and an anion exchange resin. In some embodiments, it is contacted with a cation exchange resin and then an anion exchange resin. In some embodiments, exchange of cation(s) before exchange of anion(s) can provide various benefits, e.g., reduction or prevention of formation of insoluble inorganic metal hydroxide complexes. In some embodiments, it is contacted with an anion exchange resin and then a cation exchange resin. In some embodiments, a cation exchange resin is a strong cation exchange resin. In some embodiments, an anion exchange resin is a strong anion exchange resin. In some embodiments, a composition is contacted with a cation exchange resin and an anion exchange resin simultaneously.

In some embodiments, contact with resin, e.g., ion exchange, is conducted in a batch, columnar, or continuous flow method. In some embodiments, it is conducted in a batch method. In some embodiments, it is conducted in column method. In some embodiments, it is conducted in a continuous flow method.

Purification, e.g., ion exchange, may be performed at various suitable temperatures. Useful ion exchange conditions are available and can be utilized in accordance with the present disclosure. For example, in some embodiments, ion exchange is performed at about ambient temperature. In some embodiments, ion exchange is performed at a temperature higher than ambient temperature, e.g., about 40° C.

Compositions, e.g., preparations, are typically contacted with resins for sufficient time. In some embodiments, sufficiency is assessed by extent of desired ion exchange. In some embodiments, sufficiency is assessed by extent of impurity removal. In some embodiments, sufficiency is assessed by percentage of product loss and/or recovery. In some embodiments, a time is about 1, 2, 3, 4, 5, 6, 7, 8, 9, or 10 or more hours. In some embodiments, a time is about 1 hour. In some embodiments, a time is about 2 hours. In some embodiments, a time is about 3 hours. In some embodiments, a time is about 4 hours. In some embodiments, a time is about 5 hours.

### Post-Treatment

In some embodiments, provided technologies comprises post-treatment of one or more compositions or resin after contacting a preparation with a resin. In some embodiments, post-treatment is or comprises post-recovering. In various embodiments, e.g., utilized separately or in combination with one or more other technologies provided herein, an IEX resin to which a glycol compound(s) of interest has been adsorbed (e.g., out of an aqueous initial preparation and/or pre-masking) is washed (e.g., with water [e.g., deionized water] or an alcohol such as a short-chain alcohol, e.g., ethanol or methanol), or a mixture thereof, to release the adsorbed glycol compound(s) from the resin.

Various solvent systems may be utilized to wash resin in accordance with the present disclosure. For example, in some embodiments, a solvent system is or comprises water. In some embodiments, a solvent system is or comprises an alcohol. In some embodiments, a solvent system is or comprises a short chain alcohol such as methanol or ethanol. In some embodiments, a solvent system is or comprises a water-soluble organic solvent such as tetrahydrofuran and/or acetone. In some embodiments, a solvent system is or comprises water, methanol, ethanol, isopropanol, tetrahydrofuran, acetone, or a mixture of two or more thereof. In some embodiments, a solvent system is or comprises water. In some embodiments, a solvent system is or comprises methanol. In some embodiments, a solvent system is or comprises ethanol. In some embodiments, a solvent system is or comprises isopropanol. In some embodiments, a solvent system is or comprises tetrahydrofuran. In some embodiments, a solvent system is or comprises acetone. In some embodiments, an organic solvent has a boiling point lower than water under normal pressure or reduced pressure (e.g., pressure for evaporation and/or distillation). Those skilled in the art can readily assess a solvent system, either of a single solvent or of a mixture of two or more solvents, in accordance with the present disclosure for its usefulness for washing off a glycol compound from a resin. A post-recovering solvent system is typically a pure solvent system, free of ions and compounds other than the solvent(s) therein. In some embodiments, total ion content of a post-recovering solvent system is no more than about 1000, 900, 800, 700, 600, 500, 400, 300, 200, 100, 90, 80, 70, 60, 50, 40, 30, 20, 10, 9, 8, 7, 6, 5, 4, 3, 2, or 1 ppm (wt/wt). In some embodiments, it is no more than about 100 ppm. In some embodiments, it is no more than about 50 ppm. In some embodiments, it is no more than about 10 ppm. In some embodiments, it is no more than about 5 ppm. In some embodiments, it is no more than about 1 ppm. In some embodiments, it is no more than about 0.5 ppm. In some embodiments, it is no more than about 0.1 ppm. In some embodiments, total impurity content of a post-recovering solvent system is no more than about 1000, 900, 800, 700, 600, 500, 400, 300, 200, 100, 90, 80, 70, 60, 50, 40, 30, 20, 10, 9, 8, 7, 6, 5, 4, 3, 2, or 1 ppm (wt/wt). In some embodiments, it is no more than about 100 ppm. In some embodiments, it is no more than about 50 ppm. In some embodiments, it is no more than about 10 ppm. In some embodiments, it is no more than about 5 ppm. In some embodiments, it is no more than about 1 ppm. In some embodiments, it is no more than about 0.5 ppm. In some embodiments, it is no more than about 0.1 ppm.

In some embodiments, about or at least about 1 to 100, e.g., about 1-50, 1-20, 1-10, 2-10, 5-10, or about 1, 2, 3, 4, 5, 6, 7, 8, 9, 10, 15, 20, 30, 40, 50, 60, 70, 80, 90 or 100 mL of a solvent system is applied per 1 mL of a resin (mL/mL) for post-recovering. In some embodiments, it is about 1 mL/mL. In some embodiments, it is about 2 mL/mL. In some embodiments, it is about 3 mL/mL. In some embodiments, it is about 4 mL/mL. In some embodiments, it is about 5 mL/mL. In some embodiments, it is about 6 mL/mL. In some embodiments, it is about 7 mL/mL. In some embodiments, it is about 8 mL/mL. In some embodiments, it is about 9 mL/mL. In some embodiments, it is about 10 mL/mL. In some embodiments, it is about 15 mL/mL. In some embodiments, it is about 20 mL/mL. In some embodiments, it is about 30 mL/mL. In some embodiments, it is about 40 mL/mL. In some embodiments, it is about 50 mL/mL.

In some embodiments, such post-treatment washing is performed for a period of time that is at least about 20 mins, about 30 mins, about 40 mins, about 50 mins, about 60 mins, about 70 mins, about 80 mins, about 90 mins or more. In some embodiments, such period of time is about 60 mins.

In some embodiments, post-recovering significantly reduces loss of a glycol compound. In some embodiments, it reduces the loss by about or at least about 1.5, 2, 3, 4, 5, 6, 7, 8, 9, or 10 fold (loss without post-recovering/loss with post-recovering). In some embodiments, a reduction is about or at least about 1.5 fold. In some embodiments, it is about or at least about 2 fold. In some embodiments, it is about or at least about 5 fold. In some embodiments, it is about or at least about 10 fold.

In some embodiments, both pre-masking and post-recovering are utilized and they significantly reduce loss of a glycol compound. In some embodiments, they reduce the loss by about or at least about 1.5, 2, 3, 4, 5, 6, 7, 8, 9, or 10 fold (loss without post-recovering/loss with post-recovering). In some embodiments, a reduction is about or at least about 1.5 fold. In some embodiments, it is about or at least about 2 fold. In some embodiments, it is about or at least about 5 fold. In some embodiments, it is about or at least about 10 fold.

In some embodiments, compositions from post-recovering are combined with flow-through compositions from ion exchanges (IEX). In some embodiments, flow-through compositions and/or post-recovering wash compositions are concentrated/further purified through evaporation to remove various solvents, e.g., those having lower boiling points compared to glycol compounds. In some embodiments, solvents (e.g., those present in IEX flow-through compositions and/or post-recovering wash compositions such as water, methanol, ethanol, etc.) are removed through evaporation processes. In some embodiments, evaporation is performed at reduced pressure. Those skilled in the art appreciate that various evaporation technologies are available and can be utilized in accordance with the present disclosure. In some embodiments, after evaporation compositions with about 30-95% (e.g., about 40-95%, 40-90%, 50%-90%, or 80%-95%; or about 30%, 40%, 50%, 60%, 70%, 80%, or 90%) (w/w) glycol compounds such as HDO are provided. In some embodiments, a multiple effect evaporation process optionally under vacuum is utilized. In some embodiments, evaporation removes water. In some embodiments, evaporation reduces vapor load in distillation. In some embodiments, evaporation is performed under suitable conditions under which very little or no loss of desired glycol compounds such as HDO. Alternatively or additionally, distillation is performed to further concentrate/purify glycol compounds to provide high purity preparations.

In some embodiments, provided technologies comprise polishing. In some embodiments, polishing removes salts and/or other impurities in a glycol compound composition, e.g., a HDO preparation, before distillation. In some embodiments, polishing is performed after evaporation. As those skilled in the art reading the present disclosure will appreciate, evaporation of certain solvents, e.g., water, may cause precipitation, e.g., of certain salt(s), and/or concentration of certain impurities (e.g., those that may not have been captured during IEX). In some embodiments, polishing reduces levels of one or more salts and/or impurities. Polishing can provide various benefits, improvements and/or advantages. For example, in some embodiments, polishing can provide better heating efficiency in distillation. In some embodiments, polishing reduces or removes scaling, e.g., due to the presence of salt(s) in a reboiler. Various technologies may be utilized for polishing as described herein. For example, in some embodiments, IEX resins are utilized. In some embodiments, activated carbon are utilized. In some embodiments, IEX resins are utilized as a bed. In some embodiments, both cation and anion exchange resins are utilized. In some embodiments, IEX resins are utilized as a mixed bed containing cation and anion exchange resins. In some embodiments, IEX for polishing is or comprises cation exchange. In some embodiments, IEX for polishing is or comprises anion exchange. In some embodiments, IEX for polishing is or comprises cation and anion exchange. Various IEX resins are as described herein. In some embodiments, a resin is a strong ion exchange resin. In some embodiments, a resin is a weak ion exchange resin.

### Deodorization and Decolorization

In some embodiments, a compound, e.g., a glycol compound such as HDO, is provided as a colorless liquid or solid. For example, in some embodiments, HDO is provided as a colorless solid at room temperature. In some embodiments, a method comprises removing odor or color in a composition, e.g., a HDO preparation. In some embodiments, a method comprises removing odor. In some embodiments, a method comprises removing color. In some embodiments, a method comprises removing odor and color. In some embodiments, decolorization and/or deodorization steps are included in a manufacturing process. Decolorization and deodorization may each independently be a single or multi-step process. In some embodiments, decolorization is a single step. In some embodiments, deodorization is a single step. In some embodiments, decolorization is a multistep process. In some embodiments, deodorization is a multistep process. In some embodiments, decolorization and deodorization are performed together. In some embodiments, decolorization and deodorization are a single step process.

Decolorization and/or deodorization may be independently performed at various points. For example, in some embodiments, decolorization and/or deodorization are performed after a polishing step and before distillation. In some embodiments, decolorization and/or deodorization are performed after an evaporation step and before distillation. In some embodiments, decolorization and/or deodorization are performed after a first distillation column where lower boiling compounds are removed, and before the second distillation column where a glycol compound, e.g., 1,6-HDO, can be obtained as a pure product. In some embodiments, decolorization and/or deodorization are performed after a second distillation column where a purified glycol compound, e.g., 1,6-HDO, is sent for decolorization and/or deodorization. In some embodiments, decolorization and/or deodorization are performed together or one after the other. In some embodiments, they are performed in multi-steps. In some embodiments, they are performed with other steps in between. For example, in some embodiments, deodorization is before a second distillation column whereas decolorization is after the second distillation column or vice versa.

Various technologies may be utilized for deodorization and/or decolorization in accordance with the present disclosure. Certain useful technologies are herein below as examples, e.g., hydrogenation, porous materials, etc. In some embodiments, contact with IEX can remove certain odorous and/or color materials. In some embodiments, a present disclosure comprises one or more such technologies.

### Hydrogenation

In some embodiments, a provided method comprises hydrogenation. In some embodiments, hydrogenation provides deodorization and/or decolorization. In some embodiments, a composition, e.g., a glycol compound preparation (e.g., an HDO stream), is reacted with hydrogen. In some embodiments, hydrogenation is performed in the presence of a catalyst. In some embodiments, hydrogenation is performed in the presence of a catalyst and under increased pressure. In some embodiments, hydrogenation is performed in the presence of a catalyst and under heat and pressure.

In some embodiments, a catalyst is or comprises a metal. In some embodiments, a catalyst is or comprises a metal complex. In some embodiments, a catalyst is or comprises a metal oxide. In some embodiments, a metal is palladium. In some embodiments, a catalytic is or comprises palladium. In some embodiments, a catalytic is or comprises palladium compound. In some embodiments, a catalytic is or comprises a palladium complex. In some embodiments, a metal is nickel. In some embodiments, a catalyst is Raney Nickel. In some embodiments, a metal is platinum. In some embodiments, a catalyst comprises a combination of metals, metal oxides or metal complexes.

In some embodiments, a catalyst is on a support structure. In some embodiments, a support structure is or comprises carbon. In some embodiments, a support structure is or comprises alumina. In some embodiments, a support structure is or comprises silica. In some embodiments, a support structure is or comprises a combination, e.g., of carbon, alumina and/or silica.

In some embodiments, a reactor for hydrogenation is or comprises a packed bed configuration. In some embodiments, a packed bed is or comprises random packing. In some embodiments, a packed bed is or comprises structured packing. In some embodiments, a reactor is or comprises a fluidized bed configuration. In some embodiments, a reactor is a continuous stirred tank slurry reactor. In some embodiments, a reactor is or comprises a plug flow reaction configuration.

In some embodiments, a reaction pressure is about atmospheric to about 300 bar. In some embodiments, a reaction pressure is about 1 to about 300 bar. In some embodiments, hydrogen pressure is about 1 to about 300 bar, e.g., about 1-250 bar, about 1-200 bar, about 1-150 bar, about 1-100 bar, about 1-50 bar, about 20-300 bar, about 30-300 bar, about 40-300 bar, about 50-300 bar, etc.

In some embodiments, a reaction temperature is about ambient to about 300° C. In some embodiments, it is about 200-300° C., e.g., about 25-300° C., about 30-300° C., about 40-300° C., about 50-300° C., about 25-250° C., about 25-200° C., about 25-150° C., about 25-100° C., etc.

As described herein, hydrogenation may be performed at various points. For example, in some embodiments, hydrogenation reaction is carried out after the heavies (compounds having boiling point higher than a glycol compound, e.g., HDO) are removed e.g., sugars, nitrogen containing compounds, etc. In some embodiments, hydrogenation after removal of the heavies provides benefits such as reducing or preventing fouling of catalyst. In some embodiments, hydrogenation is performed after a first distillation column or after a second distillation column, e.g., for deodorization and/or decolorization. In some embodiments, hydrogenation is performed after a first distillation column (e.g., if a glycol compound, e.g., HDO, of sufficient purity is obtained, e.g., via a middle cut fractionation product). In some embodiments, hydrogenation is performed after a second distillation column. In some embodiments, a present disclosure comprises assessing impurities transformed by hydrogenation.

### Activated Carbon

In some embodiments, a provide method comprises contacting a composition, e.g., a glycol compound preparation such as a HDO preparation, with a porous material such as activated carbon. In some embodiments, a porous material is useful for deodorization and/or decolorization. In some embodiments, a porous material is or comprises activated carbon. In some embodiments, a porous material absorbs odor and/or color. Various porous materials, e.g., activated carbon, can be utilized in accordance with the present disclosure.

In some embodiments, a composition, e.g., a glycol compound preparation (e.g., an HDO stream), is passed over a bed of activated carbon. In some embodiments, an activated carbon is added and a slurry is prepared, e.g., in a suitable container such as a stirred tank.

Contact with a porous material, e.g., activated carbon, may be performed at various points. For example, in some embodiments, contact is performed before distillation. In some embodiments, contact is performed before a first column distillation but before the next. In some embodiments, a glycol compound composition, e.g., an un-distilled HDO preparation, is contacted with a packed bed of activated carbon. Various conditions can be utilized in accordance with the present disclosure. For example, in some embodiments, pressures ranging from about 1 bar to about 4 bar and/or temperatures ranging from ambient to about 80° C. can be utilized. In some embodiments, contact is performed in a slurry composition. For example, in some embodiments, a glycol compound composition, e.g., a HDO preparation, is mixed in a stirred reactor, e.g., a stirred tank reactor, at a pressure ranging from about 1 bar to about 4 bar and/or a temperature ranging from ambient to about 80° C. In some embodiments, a slurry is filtered, e.g., using bad filter, sparkler filter, vacuum drum filter, etc., to remove activated carbon. In some embodiments, activated carbon is removed by centrifugation.

In some embodiments, contact with a porous material, e.g., activated carbon, is performed after distillation. In some embodiments, it is performed at the end for a final purification. Various technologies, e.g., those described above, may be utilized in accordance with the present disclosure. In some embodiments, a final product filter, e.g., a candle filter, is utilized, e.g., to remove carbon fines and/or blackishness.

### High Purity Preparations

The present disclosure provides technologies that achieve production of high purity preparations of glycol compound(s), and in particular of long chain glycol compound(s) (e.g., having at least 5 carbon atoms, such as having 5-12 carbon atoms), and/or in particular from aqueous initial preparations (e.g., so that a high purity aqueous preparation is achieved).

Various technologies, e.g., HPLC, GC, and/or IC (ion chromatography), may be utilized to assess a composition as described herein. For example, in some embodiments, a high purity preparation is characterized by HPLC, GC, and/or IC.

In some embodiments, a high purity preparation is an aqueous preparation; in some embodiments, a high purity preparation comprises water within a range greater than 50% and/or in an amount not more than 99%.

In some embodiments, a high purity preparation of a particular glycol compound of interest includes that compound at a level that is between about 1% and about 50% (w/w) of a preparation. In some embodiments, a level is between about 1% and about 45% (w/w). In some embodiments, a level is between about 1% and about 40% (w/w). In some embodiments, a level is between about 1% and about 35% (w/w). In some embodiments, a level is between about 1% and about 30% (w/w). In some embodiments, a level is between about 1% and about 25% (w/w). In some embodiments, a level is between about 1% and about 20% (w/w). In some embodiments, a level is between about 1% and about 15% (w/w). In some embodiments, a level is between about 1% and about 10% (w/w). In some embodiments, a level is between about 1% and about 5% (w/w). In some embodiments, a level is between about 10% and about 50% (w/w). In some embodiments, a level is between about 20% and about 50% (w/w). In some embodiments, a level is between about 30% and about 50% (w/w). In some embodiments, a level is between about 40% and about 50% (w/w). In some embodiments, a level is about 50% (w/w). In some embodiments, a level is about 45% (w/w). In some embodiments, a level is about 40% (w/w). In some embodiments, a level is about 35% (w/w). In some embodiments, a level is about 30% (w/w). In some embodiments, a level is about 25% (w/w). In some embodiments, a level is about 20% (w/w). In some embodiments, a level is about 15% (w/w). In some embodiments, a level is about 10% (w/w). In some embodiments, a level is about 5% (w/w). In some embodiments, a level is about 2% (w/w).

In some embodiments, in a high purity preparation one or more ions (e.g., inorganic ions (e.g., sulfate, phosphate, pyruvate, lithium, sodium, potassium, ammonium, calcium, magnesium, iron(II), iron(III), aluminum, chloride), organic ions (e.g., acetate, lactate, malate, succinate, maleate), amino acids (e.g., valine, and/or leucine)) and/or impurity compounds are independently absent (e.g., below detection limits of various detection technologies) or if present are independently of low levels, e.g., no more than about 10000, 9000, 8000, 7000, 6000, 5000, 4000, 3000, 2000, 1000, 900, 800, 700, 600, 500, 400, 300, 200, 100, 90, 80, 70, 60, 50, 40, 30, 20, 10, 9, 8, 7, 6, 5, 4, 3, 2, 1, 0.9, 0.8, 0.7, 0.6, 0.5, 0.4, 0.3, 0.2 or 0.1 ppm (unless otherwise noted, w/w). In some embodiments, a level is about or no more than about 10000 ppm. In some embodiments, a level is about or no more than about 9000 ppm. In some embodiments, a level is about or no more than about 8000 ppm. In some embodiments, a level is about or no more than about 7000 ppm. In some embodiments, a level is about or no more than about 6000 ppm. In some embodiments, a level is about or no more than about 5000 ppm. In some embodiments, a level is about or no more than about 4000 ppm. In some embodiments, a level is about or no more than about 3000 ppm. In some embodiments, a level is about or no more than about 2000 ppm. In some embodiments, a level is about or no more than about 1000 ppm. In some embodiments, a level is about or no more than about 900 ppm. In some embodiments, a level is about or no more than about 800 ppm. In some embodiments, a level is about or no more than about 700 ppm. In some embodiments, a level is about or no more than about 600 ppm. In some embodiments, a level is about or no more than about 500 ppm. In some embodiments, a level is about or no more than about 400 ppm. In some embodiments, a level is about or no more than about 300 ppm. In some embodiments, a level is about or no more than about 200 ppm. In some embodiments, a level is about or no more than about 100 ppm. In some embodiments, a level is about or no more than about 90 ppm. In some embodiments, a level is about or no more than about 80 ppm. In some embodiments, a level is about or no more than about 70 ppm. In some embodiments, a level is about or no more than about 60 ppm. In some embodiments, a level is about or no more than about 50 ppm. In some embodiments, a level is about or no more than about 40 ppm. In some embodiments, a level is about or no more than about 30 ppm. In some embodiments, a level is about or no more than about 20 ppm. In some embodiments, a level is about or no more than about 10 ppm. In some embodiments, a level is about or no more than about 9 ppm. In some embodiments, a level is about or no more than about 8 ppm. In some embodiments, a level is about or no more than about 7 ppm. In some embodiments, a level is about or no more than about 6 ppm. In some embodiments, a level is about or no more than about 5 ppm. In some embodiments, a level is about or no more than about 4 ppm. In some embodiments, a level is about or no more than about 3 ppm. In some embodiments, a level is about or no more than about 2 ppm. In some embodiments, a level is about or no more than about 1 ppm. In some embodiments, a level is about or no more than about 0.9 ppm. In some embodiments, a level is about or no more than about 0.8 ppm. In some embodiments, a level is about or no more than about 0.7 ppm. In some embodiments, a level is about or no more than about 0.6 ppm. In some embodiments, a level is about or no more than about 0.5 ppm. In some embodiments, a level is about or no more than about 0.4 ppm. In some embodiments, a level is about or no more than about 0.3 ppm. In some embodiments, a level is about or no more than about 0.2 ppm. In some embodiments, a level is about or no more than about 0.1 ppm. In some embodiments, provided technologies comprising pre-masking and/or post-recovering reduce levels of impurities and/or ions comparably to, no less than or more than comparable or otherwise identical technologies without pre-masking and post-recovering.

For example, in some embodiments, level of chloride ion in a high purity preparation is about or no more than about 10000 ppm. In some embodiments, it is about or no more than about 5000 ppm. In some embodiments, it is about or no more than about 2000 ppm. In some embodiments, it is about or no more than about 1000 ppm. In some embodiments, it is about or no more than about 500 ppm. In some embodiments, it is about or no more than about 200 ppm. In some embodiments, it is about or no more than about 100 ppm. In some embodiments, it is about or no more than about 50 ppm. In some embodiments, it is about or no more than about 20 ppm. In some embodiments, it is about or no more than about 10 ppm. In some embodiments, it is about or no more than about 5 ppm. In some embodiments, it is about or no more than about 1 ppm. In some embodiments, it is about or no more than about 1 ppm.

Additionally or alternatively, in some embodiments, level of sodium ion in a high purity preparation is about or no more than about 10000 ppm. In some embodiments, it is about or no more than about 5000 ppm. In some embodiments, it is about or no more than about 2000 ppm. In some embodiments, it is about or no more than about 1000 ppm. In some embodiments, it is about or no more than about 500 ppm. In some embodiments, it is about or no more than about 200 ppm. In some embodiments, it is about or no more than about 100 ppm. In some embodiments, it is about or no more than about 50 ppm. In some embodiments, it is about or no more than about 20 ppm. In some embodiments, it is about or no more than about 10 ppm. In some embodiments, it is about or no more than about 5 ppm. In some embodiments, it is about or no more than about 1 ppm. In some embodiments, it is about or no more than about 1 ppm.

Additionally or alternatively, in some embodiments, level of potassium ion in a high purity preparation is about or no more than about 10000 ppm. In some embodiments, it is about or no more than about 5000 ppm. In some embodiments, it is about or no more than about 2000 ppm. In some embodiments, it is about or no more than about 1000 ppm. In some embodiments, it is about or no more than about 500 ppm. In some embodiments, it is about or no more than about 200 ppm. In some embodiments, it is about or no more than about 100 ppm. In some embodiments, it is about or no more than about 50 ppm. In some embodiments, it is about or no more than about 20 ppm. In some embodiments, it is about or no more than about 10 ppm. In some embodiments, it is about or no more than about 5 ppm. In some embodiments, it is about or no more than about 1 ppm. In some embodiments, it is about or no more than about 1 ppm.

Additionally or alternatively, in some embodiments, level of magnesium ion in a high purity preparation is about or no more than about 10000 ppm. In some embodiments, it is about or no more than about 5000 ppm. In some embodiments, it is about or no more than about 2000 ppm. In some embodiments, it is about or no more than about 1000 ppm. In some embodiments, it is about or no more than about 500 ppm. In some embodiments, it is about or no more than about 200 ppm. In some embodiments, it is about or no more than about 100 ppm. In some embodiments, it is about or no more than about 50 ppm. In some embodiments, it is about or no more than about 20 ppm. In some embodiments, it is about or no more than about 10 ppm. In some embodiments, it is about or no more than about 5 ppm. In some embodiments, it is about or no more than about 1 ppm. In some embodiments, it is about or no more than about 1 ppm.

Additionally or alternatively, in some embodiments, level of H₂PO₄⁻, HPO₄²⁻, and/or PO₄³⁻ ions in a high purity preparation is about or no more than about 10000 ppm. In some embodiments, it is about or no more than about 5000 ppm. In some embodiments, it is about or no more than about 2000 ppm. In some embodiments, it is about or no more than about 1000 ppm. In some embodiments, it is about or no more than about 500 ppm. In some embodiments, it is about or no more than about 200 ppm. In some embodiments, it is about or no more than about 100 ppm. In some embodiments, it is about or no more than about 50 ppm. In some embodiments, it is about or no more than about 20 ppm. In some embodiments, it is about or no more than about 10 ppm. In some embodiments, it is about or no more than about 5 ppm. In some embodiments, it is about or no more than about 1 ppm. In some embodiments, it is about or no more than about 1 ppm.

Additionally or alternatively, in some embodiments, level of sulfate ion in a high purity preparation is about or no more than about 10000 ppm. In some embodiments, it is about or no more than about 5000 ppm. In some embodiments, it is about or no more than about 2000 ppm. In some embodiments, it is about or no more than about 1000 ppm. In some embodiments, it is about or no more than about 500 ppm. In some embodiments, it is about or no more than about 200 ppm. In some embodiments, it is about or no more than about 100 ppm. In some embodiments, it is about or no more than about 50 ppm. In some embodiments, it is about or no more than about 20 ppm. In some embodiments, it is about or no more than about 10 ppm. In some embodiments, it is about or no more than about 5 ppm. In some embodiments, it is about or no more than about 1 ppm. In some embodiments, it is about or no more than about 1 ppm.

Additionally or alternatively, in some embodiments, level of pyruvate in a high purity preparation is about or no more than about 10000 ppm. In some embodiments, it is about or no more than about 5000 ppm. In some embodiments, it is about or no more than about 2000 ppm. In some embodiments, it is about or no more than about 1000 ppm. In some embodiments, it is about or no more than about 500 ppm. In some embodiments, it is about or no more than about 200 ppm. In some embodiments, it is about or no more than about 100 ppm. In some embodiments, it is about or no more than about 50 ppm. In some embodiments, it is about or no more than about 20 ppm. In some embodiments, it is about or no more than about 10 ppm. In some embodiments, it is about or no more than about 5 ppm. In some embodiments, it is about or no more than about 1 ppm. In some embodiments, it is about or no more than about 1 ppm.

In some embodiments, a high purity preparation is characterized by a reduced concentration of one or more ions (e.g., inorganic ions (e.g., sulfate, phosphate, pyruvate, lithium, sodium, potassium, ammonium, calcium, magnesium, iron(II), iron(III), aluminum, chloride), organic ions (e.g., acetate, lactate, malate, succinate, maleate), amino acids (e.g., valine, and/or leucine)) and/or impurity compounds and/or impurity compounds relative to that present in an aqueous initial preparation (e.g., from which it is prepared) or a composition before ion exchanges (a pre-IEX composition). In some embodiments, a high purity preparation is characterized by a reduced concentration of one or more of the following ions: sulfate, phosphate, pyruvate, lithium, sodium, potassium, ammonium, calcium, magnesium, iron(II), iron(III), aluminum, chloride, acetate, lactate, malate, succinate, maleate, valine, leucine relative to that present in an aqueous initial preparation (e.g., from which it is prepared). In some embodiments, a high purity preparation is characterized by a reduced concentration of chloride ion relative to that present in an aqueous initial preparation (e.g., from which it is prepared). In some embodiments, a high purity preparation is characterized by a reduced concentration of sodium ion relative to that present in an aqueous initial preparation (e.g., from which it is prepared). In some embodiments, a high purity preparation is characterized by a reduced concentration of potassium ion relative to that present in an aqueous initial preparation (e.g., from which it is prepared). In some embodiments, a high purity preparation is characterized by a reduced concentration of magnesium ion relative to that present in an aqueous initial preparation (e.g., from which it is prepared). In some embodiments, a high purity preparation is characterized by a reduced concentration of sulfate ion relative to that present in an aqueous initial preparation (e.g., from which it is prepared). In some embodiments, a high purity preparation is characterized by a reduced concentration of phosphate ion relative to that present in an aqueous initial preparation (e.g., from which it is prepared). In some embodiments, a high purity preparation is characterized by a reduced concentration of pyruvate relative to that present in an aqueous initial preparation (e.g., from which it is prepared). In some embodiments, a concentration or level is reduced by about or at least about 50%. In some embodiments, it is reduced by about or at least about 60%. In some embodiments, it is reduced by about or at least about 70%. In some embodiments, it is reduced by about or at least about 75%. In some embodiments, it is reduced by about or at least about 80%. In some embodiments, it is reduced by about or at least about 85%. In some embodiments, it is reduced by about or at least about 90%. In some embodiments, it is reduced by about or at least about 95%. In some embodiments, it is reduced by about or at least about 96%. In some embodiments, it is reduced by about or at least about 97%. In some embodiments, it is reduced by about or at least about 98%. In some embodiments, it is reduced by about or at least about 99%. In some embodiments, it is reduced by about or at least about 99.5%. In some embodiments, it is reduced by about or at least about 99.9% (e.g., from 1000 ppm to no more than 1 ppm).

Various technologies, e.g., HPLC, IC, or GC coupled with UV, MS, etc., are available for assessing levels of ions, impurities, glycol compounds, etc. and can be utilized in accordance with the present disclosure.

In some embodiments, a high purity preparation is a commercial scale preparation. In some embodiments, a high purity preparation is a large-scale preparation with a volume range of, e.g., about 1 kL to about 1000 kL. In some embodiments, a volume is between about 10 kL and about 800 kL. In some embodiments, a volume is between about 10 kL and about 600 kL. In some embodiments, a volume is between about 10 kL and about 400 kL. In some embodiments, a volume is between about 10 kL and about 200 kL. In some embodiments, a volume is between about 10 kL and about 100 kL. In some embodiments, a volume is between about 10 kL and about 80 kL. In some embodiments, a volume is between about 10 kL and about 70 kL. In some embodiments, a volume is between about 10 kL and about 60 kL. In some embodiments, a volume is between about 10 kL and about 50 kL. In some embodiments, a volume is between about 10 kL and about 40 kL. In some embodiments, a volume is between about 10 kL and about 30 kL. In some embodiments, a volume is between about 10 kL and about 20 kL. In some embodiments, a volume is between about 10 kL and about 15 kL. In some embodiments, a volume is between about 20 kL and about 800 kL. In some embodiments, a volume is between about 50 kL and about 800 kL. In some embodiments, a volume is between about 100 kL and about 800 kL. In some embodiments, a volume is between about 300 kL and about 800 kL. In some embodiments, a volume is between about 500 kL and about 800 kL. In some embodiments, a volume is between about 20 kL and about 100 kL. In some embodiments, a volume is between about 30 kL and about 80 kL. In some embodiments, a volume is between about 40 kL and about 70 kL. In some embodiments, a volume is about 10 kL, 20 kL, 30 kL, 40 kL, 50 kL, 60 kL, 70 kL, or 80 kL. In some embodiments, a volume is about 10 kL. In some embodiments, a volume is about 20 kL. In some embodiments, a volume is about 30 kL. In some embodiments, a volume is about 40 kL. In some embodiments, a volume is about 50 kL. In some embodiments, a volume is about 60 kL. In some embodiments, a volume is about 70 kL. In some embodiments, a volume is about 80 kL. In some embodiments, a volume is about 90 kL. In some embodiments, a volume is about 100 kL. In some embodiments, a volume is about 200 kL. In some embodiments, a volume is about 300 kL. In some embodiments, a volume is about 400 kL. In some embodiments, a volume is about 500 kL. In some embodiments, a volume is about 600 kL. In some embodiments, a volume is about 700 kL. In some embodiments, a volume is about 800 kL. In some embodiments, a volume is about 900 kL. In some embodiments, a volume is about 1000 kL.

In some embodiments, a high purity preparation is generated by performance of an IEX purification as described herein (e.g., without further processing step(s)).

In some embodiments, distillation technologies are utilized to remove certain impurities. For example, in some embodiments, distillation is utilized to remove impurities with boiling points under certain pressures that are lower than a glycol compound. In some embodiments, a glycol compound is distilled to improve purity. In some embodiments, water is removed through distillation.

In some embodiments, a distillation technology is thin-film distillation. For example, in some embodiments, water is removed from a glycol compound preparation, e.g., a 1,6-HDO preparation, a thin-film distillation apparatus at about 70° C. In some embodiments, distillation is performed under continuous feeding operating conditions. In some embodiments, water content of a composition, e.g., a 1,6-HDO preparation, is about or no more than about 500, 400, 300, 200, 100, 90, 80, 70, 60, 50, 40, 30, 20, or 10 ppm.

In some embodiments, distillation may be performed at various conditions so that impurities of different properties, e.g., boiling points under certain pressures, can be removed. For example, in some embodiments, water and/or other impurities with relatively low boiling points are first removed at relatively low temperatures, e.g., about 70° C. Impurities with relatively higher boiling points, e.g., higher than water but still lower than desired compounds (e.g., 1,6-HDO) are removed at relatively higher temperatures. For example, in some embodiments, distillation is performed on a 1,6-HDO composition such that the top of a distillation column is maintained at about 240° C. to remove impurities.

In some embodiments, after removal of impurities, compounds are purified by distillation. For example, in some embodiments, 1,6-HDO is distilled out by maintaining the bottom of a distillation column at 260° C. In some embodiments, collected 1,6-HDO has a purity of about 95% or more (w/w) by HPLC and/or GC.

Those skilled in the art appreciate various distillation technologies can be utilized in accordance with the present disclosure. For example, in some embodiments, a distillation apparatus is equipped with an Oldershaw column. In some embodiments, a composition is continuously supplied to a distillation apparatus.

Various distillation technologies may be utilized in accordance with the present disclosure. In some embodiments, a distillation method is continuous distillation. In some embodiments, a distillation method is batch distillation.

In some embodiments, a preparation of a glycol compound has a purity of about 90%, 91%, 92%, 93%, 94%, 95%, 96%, 97%, 98%, 99%, 99.5% or more (w/w). In some embodiments, a purity is about 90% or more. In some embodiments, it is about 91% or more. In some embodiments, it is about 92% or more. In some embodiments, it is about 93% or more. In some embodiments, it is about 94% or more. In some embodiments, it is about 95% or more. In some embodiments, it is about 96% or more. In some embodiments, it is about 97% or more. In some embodiments, it is about 98% or more. In some embodiments, it is about 99% or more. In some embodiments, it is about 99.5% or more.

### Products

Glycol compounds as described herein (e.g., long chain glycol compounds such as glycol compounds containing at least 5 carbon atoms, such as 5-12 carbon atoms) are well known to be useful in a variety of commercial contexts.

To give but a few examples, 1,6-hexanediol (HDO) is useful, among other things, in production of polyurethanes, coatings (e.g., automotive coatings), acrylates, adhesives, polyester resins, plasticizers, chemical probes (see, e.g., Kroschwald, et al., Hexanediol: a chemical probe to investigate the material properties of membrane-less compartments, Matters 3 (5), e201702000010; Sabari, et al., Coactivator condensation at super-enhancers links phase separation and gene control, Science, 2018 Jul. 27; 361(6400):eaar3958), etc. 1,5-Pentanediol is useful, among other things, in production of polyester plastics, polyurethanes, various pharmaceuticals, inks and coatings, plasticizers, solvent and industrial chemicals, etc. See, for example, Huber et al., US Department of Energy Bioenergy Technologies Office 2017 Project Peer Review.

In some embodiments, HDO is used for production of macrodiols, for example, adipate esters and polycarbonate diols used in, e.g., elastomers and polyurethane dispersions (e.g., for parquet flooring and leather coatings). Through traditional chemical or through biosynthesis processes or combinations thereof, 6-hydroxy hexanoic acid can be cyclized to make ε-caprolactone which can then be aminated to make ε-caprolactam. Through traditional chemical or through biosynthesis processes or combinations thereof, 6-hydroxy hexanoic acid can be aminated to make 6-amino hexanoic acid, which can then be cyclized to make ε-caprolactam. ε-Caprolactam, among other things, can be used for the production of Nylon6, a widely used polymer in many different industries. ε-Caprolactone can be polymerized to make polycaprolactone (PCL) a biodegradable polyester with various applications including for the production of specialty polyurethanes.

Various 2-ketocarboxylic acids are useful for various industrial relevant chemicals and pharmaceuticals.

In some embodiments, such chemicals and pharmaceuticals, or intermediates thereof, are amino acids or α-hydroxy carboxylic acids. In some embodiments, glycol compounds purified as described herein are utilized to manufacture polyesters, polyester polyols, polyurethane, nylon (e.g., from adipic acid), polycarbonate diols (e.g., from HDO or 1,5-pentanediol, etc.), diacrylate esters (e.g., from HDO or 1,5-pentanediol, etc.), diglycidyl ethers (e.g., from HDO or 1,5-pentanediol, etc.), etc.

In some embodiments, high purity preparations of glycol compound(s) as described herein (e.g., long chain glycol compounds such as those containing at least 5 carbon atoms, e.g., 5-12 carbon atoms) may be employed in the manufacturing of lubricants, adhesives, polymers, textiles, coatings, fuel and fuel additives, and other value-added products.

High purity preparations of glycol compounds of the present disclosure can provide various advantages. For example, in some embodiments, their high purity provides high efficiency production, low cost of goods, and/or high purity products; alternatively or additionally, in some embodiments, when utilized for preparing polymers, high purity preparations of glycol compounds of the present disclosure can produce polymers with high levels of polymerization.

In some embodiments, the present disclosure provides the following Embodiments as examples:
1. A method of purifying an aqueous initial preparation comprising a glycol compound,
   wherein the glycol compound has 5 to 12 carbon atoms, and
   wherein the aqueous initial preparation comprises greater than 50% water; the method comprising:
   contacting the aqueous initial preparation with an ion exchange resin, wherein the resin is a hydrophilic resin.
2. A method of purifying an aqueous initial preparation comprising a glycol compound, comprising contacting the aqueous initial preparation with an ion exchange resin, wherein the base polymer of the resin is a hydrophilic polymer.
3. The method of any one of the preceding Embodiments, wherein the hydrophilic resin is a polyacrylic resin.
4. The method of Embodiment 3, wherein the polyacrylic resin is a strong anionic exchange resin.
5. The method of Embodiment 3, wherein the polyacrylic resin is a weak anionic exchange resin.
6. The method of Embodiment 3, wherein the polyacrylic resin is a weak cationic exchange resin.
7. The method of any one of the preceding Embodiments, wherein the hydrophilic resin is a polymethacrylic resin.
8. The method of Embodiment 7, wherein the polyacrylic resin is a strong anionic exchange resin.
9. The method of Embodiment 7, wherein the polyacrylic resin is a weak anionic exchange resin.
10. The method of Embodiment 7, wherein the polyacrylic resin is a weak cationic exchange resin.
11. The method of any one of the preceding Embodiments, wherein the polymethacrylic resin is a polyacrylic resin.
12. The method of Embodiment 11, wherein the polyacrylic resin is a strong anionic exchange resin.
13. The method of Embodiment 11, wherein the polyacrylic resin is a weak anionic exchange resin.
14. The method of Embodiment 11, wherein the polyacrylic resin is a weak cationic exchange resin.
15. The method of any one of the preceding Embodiments, further comprising contacting the ion exchange resin with a pre-masking preparation comprising the glycol compound prior to contacting the aqueous initial preparation with the ion exchange resin.
16. A method of purifying an aqueous initial preparation comprising a glycol compound, comprising contacting an ion exchange resin with a pre-masking preparation comprising the glycol compound, and contacting the aqueous initial preparation with the pre-masked ion exchange resin.
17. A method of purifying an aqueous initial preparation comprising a glycol compound, comprising contacting an ion exchange resin with a pre-masking preparation comprising the glycol compound, and contacting the aqueous initial preparation with the pre-masked ion exchange resin.
18. The method of any one of the preceding Embodiments, further comprising contacting the ion exchange resin with a post-recovering solvent system after contacting the resin with the aqueous initial preparation.
19. A method of purifying an aqueous initial preparation comprising a glycol compound, comprising contacting an aqueous initial preparation with an ion exchange resin, and contacting the ion exchange resin with a post-recovering solvent system after contacting the aqueous initial preparation with the ion exchange resin.
20. A method of purifying an aqueous initial preparation comprising a glycol compound, comprising contacting an ion exchange resin with a pre-masking preparation comprising the glycol compound, contacting an aqueous initial preparation with the pre-masked ion exchange resin, and contacting the ion exchange resin with a post-recovering solvent system after contacting the aqueous initial preparation with the pre-masked ion exchange resin.
21. The method of any one of the preceding Embodiments, wherein the aqueous initial preparation comprises greater than about 50% water.
22. The method of any one of the preceding Embodiments, wherein water is present in the preparation in an amount within a range of 51% to 99%.
23. The method of any one of the preceding Embodiments, wherein the aqueous initial preparation comprises greater than about 70% water.
24. The method of any one of the preceding Embodiments, wherein the aqueous initial preparation comprises greater than about 90% water.
25. The method of any one of the preceding Embodiments, wherein the aqueous initial preparation comprises one or more ions and/or impurities independently at about or at least about 50, 60, 70, 80, 90, 100, 150, 200, 300, 400, 500, 600, 700, 800, 900, 1000, 1500, 2000, 3000, 4000, 5000, 6000, 7000, 8000, or 10000 ppm (w/w).
26. The method of any one of the preceding Embodiments, wherein the aqueous initial preparation comprises K⁺ at about or at least about 3000 ppm (w/w).
27. The method of any one of the preceding Embodiments, wherein the aqueous initial preparation comprises Mg²⁺ at about or at least about 90 ppm (w/w).
28. The method of any one of the preceding Embodiments, wherein the aqueous initial preparation comprises pyruvate at about or at least about 3000 ppm (w/w).
29. The method of any one of the preceding Embodiments, wherein the aqueous initial preparation comprises SO₄²⁻ at about or at least about 350 ppm (w/w).
30. The method of any one of the preceding Embodiments, wherein the aqueous initial preparation comprises phosphate (H₂PO₄⁻, HPO₄²⁻, and/or PO₄³⁻) at about or at least about 2000 ppm (w/w).
31. The method of any one of the preceding Embodiments, comprising contacting an aqueous initial preparation with an acidic resin.
32. The method of any one of the preceding Embodiments, comprising contacting an aqueous initial preparation with a strong acidic resin.
33. The method of any one of the preceding Embodiments, comprising contacting an aqueous initial preparation with a weak acidic resin.
34. The method of any one of Embodiments 31-33, wherein the base polymer of the resin is hydrophobic.
35. The method of Embodiment 34, wherein the resin is a polystyrene resin.
36. The method of any one of Embodiments 31-33, wherein the base polymer of the resin is hydrophilic.
37. The method of Embodiment 36, wherein the resin is a polyacrylic resin.
38. The method of Embodiment 36, wherein the resin is a polymethacrylic resin.
39. The method of any one of Embodiments 1-31, comprising contacting an aqueous initial preparation with a SK1BH resin.
40. The method of any one of Embodiments 1-31, comprising contacting an aqueous initial preparation with a WK60L resin.
41. The method of any one of Embodiments 1-31, comprising contacting an aqueous initial preparation with a WK10 resin
42. The method of any one of Embodiments 31-41, wherein the resin is pre-conditioned.
43. The method of any one of Embodiments 31-42, comprising contacting the ion exchange resin with a pre-masking preparation comprising the glycol compound prior to contacting the ion exchange resin with an aqueous initial preparation.
44. The method of any one of Embodiments 31-42, comprising contacting the ion exchange resin with a post-recovering solvent system after contacting the ion exchange resin with an aqueous initial preparation.
45. The method of any one of the preceding Embodiments, comprising contacting an aqueous initial preparation with a basic resin.
46. The method of any one of the preceding Embodiments, comprising contacting an aqueous initial preparation with a strong basic resin.
47. The method of any one of the preceding Embodiments, comprising contacting an aqueous initial preparation with a weak basic resin.
48. The method of any one of Embodiments 45-47, wherein the base polymer of the resin is hydrophobic.
49. The method of Embodiment 48, wherein the resin is a polystyrene resin.
50. The method of any one of Embodiments 45-47, wherein the base polymer of the resin is hydrophilic.
51. The method of Embodiment 50, wherein the resin is a polyacrylic resin.
52. The method of Embodiment 50, wherein the resin is a polymethacrylic resin.
53. The method of any one of Embodiments 1-45, comprising contacting an aqueous initial preparation with a SCA10AOH resin.
54. The method of any one of Embodiments 1-45, comprising contacting an aqueous initial preparation with a WA21J resin.
55. The method of any one of Embodiments 1-45, comprising contacting an aqueous initial preparation with a HPR4580 Cl resin.
56. The method of any one of Embodiments 1-45, comprising contacting an aqueous initial preparation with a SCAV4 Cl resin.
57. The method of any one of Embodiments 1-45, comprising contacting an aqueous initial preparation with an IRA67 resin.
58. The method of any one of 45-57, wherein the resin is pre-conditioned.
59. The method of any one of Embodiments 45-58, comprising contacting the ion exchange resin with a pre-masking preparation comprising the glycol compound prior to contacting the ion exchange resin with an aqueous initial preparation.
60. The method of any one of Embodiments 45-59, comprising contacting the ion exchange resin with a post-recovering solvent system after contacting the ion exchange resin with an aqueous initial preparation.
61. The method of any one of the preceding Embodiments, wherein the resin is characterized by an average cross-linking within a range of 1% to 25%.
62. The method of any one of the preceding Embodiments, wherein the resin is characterized by an average functional group density within a range of 0.1 to 4.0 (eq/L).
63. The method of any one of the preceding Embodiments, wherein the resin is characterized by an average particle size within a range of 100 μm to 1500 μm.
64. The method of any one of the preceding Embodiments, wherein the resin is resin is characterized as type 1 or type 2.
65. The method of any one of the preceding Embodiments, wherein concentration of the glycol compound in the pre-masking preparation is about or no more than about 50% of the glycol compound (wt %).
66. The method of any one of the preceding Embodiments, wherein concentration of the glycol compound in the pre-masking preparation is about or no more than about 30% of the glycol compound (wt %).
67. The method of any one of the preceding Embodiments, wherein concentration of the glycol compound in the pre-masking preparation is about or no more than about 20% of the glycol compound (wt %).
68. The method of any one of the preceding Embodiments, wherein concentration of the glycol compound in the pre-masking preparation is about or no more than about 10% of the glycol compound (wt %).
69. The method of any one of the preceding Embodiments, wherein concentration of the glycol compound in the pre-masking preparation is about or no more than about 5% of the glycol compound (wt %).
70. The method of any one of the preceding Embodiments, wherein concentration of the glycol compound in the pre-masking preparation is about or no more than about 2% of the glycol compound (wt %).
71. The method of any one of the preceding Embodiments, wherein concentration of the glycol compound in the pre-masking preparation is about or no more than about 0.1% to 2% of the glycol compound (wt %).
72. The method of any one of the preceding Embodiments, wherein concentration of the glycol compound in the pre-masking preparation is about or no more than about 1% of the glycol compound (wt %).
73. The method of any one of the preceding Embodiments, wherein concentration of the glycol compound in the pre-masking preparation is about or no more than about 0.3% to 0.7% of the glycol compound (wt %).
74. The method of any one of the preceding Embodiments, wherein concentration of the glycol compound in the pre-masking preparation is about or no more than about 0.5% of the glycol compound (wt %).
75. The method of any one of the preceding Embodiments, wherein concentration of the glycol compound in the pre-masking preparation is about or no more than about 0.2% of the glycol compound (wt %).
76. The method of any one of the preceding Embodiments, wherein concentration of the glycol compound in the pre-masking preparation is about or no more than about 0.1% of the glycol compound (wt %).
77. The method of any one of the preceding Embodiments, wherein for each contacting of an ion exchange resin with a pre-masking preparation, about or at least about 1-1000, e.g., about 1-500, 1-200, 1-100, 1-50, 1-20, 1-10, or about 1, 2, 5, 10, 15, 20, 25, 30, 40, 50, 60, 70, 80, 90, 100, 150, 200, 300, 400, 500, 600, 700, 800, 900 or 1000 mg of the glycol compound is utilized per mL of resin.
78. The method of Embodiment 77, wherein about 1-50 mg of the glycol compound is utilized per mL of resin.
79. The method of Embodiment 77, wherein about 20 mg of the glycol compound is utilized per mL of resin.
80. The method of any one of the preceding Embodiments, wherein for each pre-masking preparation, one or more ions or impurities independently have a lower level in the pre-masking preparation than the aqueous initial preparation.
81. The method of any one of the preceding Embodiments, wherein each pre-masking preparation is substantially free of one or more ions and/or impurities present in the aqueous initial preparation.
82. The method of any one of the preceding Embodiments, wherein each pre-masking preparation is substantially free of all ions and/or impurities present in the aqueous initial preparation.
83. The method of any one of the preceding Embodiments, wherein the solvent of the pre-masking preparation is water.
84. The method of any one of the preceding Embodiments, wherein for each contacting of the ion exchange resin with a post-recovering solvent system, about or at least about 1 to 100, e.g., about 1-50, 1-20, 1-10, 2-10, 5-10, or about 1, 2, 3, 4, 5, 6, 7, 8, 9, 10, 15, 20, 30, 40, 50, 60, 70, 80, 90 or 100 mL of a post-recovering solvent system is independently applied per 1 mL of a resin.
85. The method of any one of the preceding Embodiments, wherein for each contacting of the ion exchange resin with a post-recovering solvent system, about 1-10 mL of a post-recovering solvent system is independently applied per 1 mL of a resin.
86. The method of any one of the preceding Embodiments, wherein a post-recovering solvent system comprises water.
87. The method of any one of the preceding Embodiments, wherein a post-recovering solvent system comprises an alcohol.
88. The method of any one of the preceding Embodiments, wherein a post-recovering solvent system comprises a short-chain alcohol.
89. The method of any one of the preceding Embodiments, wherein a post-recovering solvent system comprises methanol.
90. The method of any one of the preceding Embodiments, wherein a post-recovering solvent system comprises ethanol.
91. The method of any one of the preceding Embodiments, wherein a post-recovering solvent system comprises an organic solvent soluble in water.
92. The method of any one of the preceding Embodiments, wherein a post-recovering solvent system comprises THF.
93. The method of any one of the preceding Embodiments, wherein a post-recovering solvent system comprises acetone.
94. The method of any one of Embodiments 1-85, wherein a post-recovering solvent system is water.
95. The method of any one of Embodiments 1-85, wherein a post-recovering solvent system is an alcohol.
96. The method of any one of Embodiments 1-85, wherein a post-recovering solvent system is a short-chain alcohol.
97. The method of any one of Embodiments 1-85, wherein a post-recovering solvent system is methanol.
98. The method of any one of Embodiments 1-85, wherein a post-recovering solvent system is ethanol.
99. The method of any one of Embodiments 1-85, wherein a post-recovering solvent system is a mixture of two or more solvent selected from water and short-chain alcohols.
100. The method of any one of the preceding Embodiments, wherein the total ion content of each post-recovering solvent system is independently no more than about 1000, 900, 800, 700, 600, 500, 400, 300, 200, 100, 90, 80, 70, 60, 50, 40, 30, 20, 10, 9, 8, 7, 6, 5, 4, 3, 2, or 1 ppm (wt/wt).
101. The method of any one of the preceding Embodiments, wherein the total ion content of each post-recovering solvent system is independently no more than about 100 ppm (wt/wt).
102. The method of any one of the preceding Embodiments, wherein the total ion content of each post-recovering solvent system is independently no more than about 10 ppm (wt/wt).
103. The method of any one of the preceding Embodiments, wherein each post-recovering solvent system is substantially free of ions and compounds other than the solvent(s).
104. The method of any one of the preceding Embodiments, wherein for each contacting of the ion exchange resin with a post-recovering solvent system, about or at least about 1 to 100, e.g., about 1-50, 1-20, 1-10, 2-10, 5-10, or about 1, 2, 3, 4, 5, 6, 7, 8, 9, 10, 15, 20, 30, 40, 50, 60, 70, 80, 90 or 100 mL of a solvent system is applied per 1 mL of a resin (mL/mL).
105. The method of Embodiment 104, wherein for each contacting of the ion exchange resin with a post-recovering solvent system, about 1-20 mL of a solvent system is applied per 1 mL of a resin (mL/mL).
106. The method of Embodiment 104, wherein for each contacting of the ion exchange resin with a post-recovering solvent system, about 1-10 mL of a solvent system is applied per 1 mL of a resin (mL/mL).
107. The method of any one of the preceding Embodiments, wherein after contacting an aqueous initial preparation with the resin(s), concentrations of one or more ions (e.g., inorganic ions (e.g., sulfate, phosphate, pyruvate, lithium, sodium, potassium, ammonium, calcium, magnesium, iron(II), iron(III), aluminum, chloride), and organic ions (e.g., acetate, lactate, malate, succinate, maleate)) are independently no more than about 1000, 900, 800, 700, 600, 500, 400, 300, 200, 100, 90, 80, 70, 60, 50, 40, 30, 20, 10, 9, 8, 7, 6, 5, 4, 3, 2, 1, 0.9, 0.8, 0.7, 0.6, 0.5, 0.4, 0.3, 0.2 or 0.1 ppm (w/w).
108. The method of any one of the preceding Embodiments, wherein a resin is in the shape of a powder.
109. The method of any one of the preceding Embodiments, wherein a resin is spherical.
110. The method of any one of the preceding Embodiments, wherein a resin is a membrane.
111. The method of any one of the preceding Embodiments, wherein a contact with a resin is performed in a batch method.
112. The method of any one of the preceding Embodiments, wherein a contact with a resin is performed in a column.
113. The method of any one of the preceding Embodiments, wherein a contact with a resin is performed in a continuous flow method.
114. The method of any one of the preceding Embodiments, wherein after contacting an aqueous initial preparation with the resin(s), concentrations of one or more ions (e.g., inorganic ions (e.g., sulfate, phosphate, pyruvate, lithium, sodium, potassium, ammonium, calcium, magnesium, iron(II), iron(III), aluminum, chloride), and organic ions (e.g., acetate, lactate, malate, succinate, maleate)) are independently no more than about 100 ppm (w/w).
115. The method of any one of the preceding Embodiments, wherein after contacting an aqueous initial preparation with the resin(s), the concentration of K⁺ is no more than 200 ppm.
116. The method of any one of the preceding Embodiments, wherein after contacting an aqueous initial preparation with the resin(s), the concentration of K⁺ is no more than 100 ppm.
117. The method of any one of the preceding Embodiments, wherein after contacting an aqueous initial preparation with the resin(s), the concentration of K⁺ is no more than 50 ppm.
118. The method of any one of the preceding Embodiments, wherein after contacting an aqueous initial preparation with the resin(s), the concentration of K⁺ is no more than 20 ppm.
119. The method of any one of the preceding Embodiments, wherein after contacting an aqueous initial preparation with the resin(s), the concentration of K⁺ is no more than 10 ppm.
120. The method of any one of the preceding Embodiments, wherein after contacting an aqueous initial preparation with the resin(s), the concentration of K⁺ is no more than 5 ppm.
121. The method of any one of the preceding Embodiments, wherein after contacting an aqueous initial preparation with the resin(s), the concentration of K⁺ is no more than 1 ppm.
122. The method of any one of the preceding Embodiments, wherein after contacting an aqueous initial preparation with the resin(s), the concentration of K⁺ is no more than 0.5 ppm.
123. The method of any one of the preceding Embodiments, wherein after contacting an aqueous initial preparation with the resin(s), the amount of K⁺ is reduced at about or at least about 90% compared to the aqueous initial preparation.
124. The method of any one of the preceding Embodiments, wherein after contacting an aqueous initial preparation with the resin(s), the amount of K⁺ is reduced at about or at least about 99% compared to the aqueous initial preparation.
125. The method of any one of the preceding Embodiments, wherein after contacting an aqueous initial preparation with the resin(s), the concentration of Mg²⁺ is no more than 200 ppm.
126. The method of any one of the preceding Embodiments, wherein after contacting an aqueous initial preparation with the resin(s), the concentration of Mg²⁺ is no more than 100 ppm.
127. The method of any one of the preceding Embodiments, wherein after contacting an aqueous initial preparation with the resin(s), the concentration of Mg²⁺ is no more than 50 ppm.
128. The method of any one of the preceding Embodiments, wherein after contacting an aqueous initial preparation with the resin(s), the concentration of Mg²⁺ is no more than 20 ppm.
129. The method of any one of the preceding Embodiments, wherein after contacting an aqueous initial preparation with the resin(s), the concentration of Mg²⁺ is no more than 10 ppm.
130. The method of any one of the preceding Embodiments, wherein after contacting an aqueous initial preparation with the resin(s), the concentration of Mg²⁺ is no more than 5 ppm.
131. The method of any one of the preceding Embodiments, wherein after contacting an aqueous initial preparation with the resin(s), the concentration of Mg²⁺ is no more than 1 ppm.
132. The method of any one of the preceding Embodiments, wherein after contacting an aqueous initial preparation with the resin(s), the concentration of Mg²⁺ is no more than 0.5 ppm.
133. The method of any one of the preceding Embodiments, wherein after contacting an aqueous initial preparation with the resin(s), the amount of Mg²⁺ is reduced at about or at least about 90% compared to the aqueous initial preparation.
134. The method of any one of the preceding Embodiments, wherein after contacting an aqueous initial preparation with the resin(s), the amount of Mg²⁺ is reduced at about or at least about 99% compared to the aqueous initial preparation.
135. The method of any one of the preceding Embodiments, wherein after contacting an aqueous initial preparation with the resin(s), the concentration of pyruvate is no more than 200 ppm.
136. The method of any one of the preceding Embodiments, wherein after contacting an aqueous initial preparation with the resin(s), the concentration of pyruvate is no more than 100 ppm.
137. The method of any one of the preceding Embodiments, wherein after contacting an aqueous initial preparation with the resin(s), the concentration of pyruvate is no more than 50 ppm.
138. The method of any one of the preceding Embodiments, wherein after contacting an aqueous initial preparation with the resin(s), the concentration of pyruvate is no more than 20 ppm.
139. The method of any one of the preceding Embodiments, wherein after contacting an aqueous initial preparation with the resin(s), the concentration of pyruvate is no more than 10 ppm.
140. The method of any one of the preceding Embodiments, wherein after contacting an aqueous initial preparation with the resin(s), the concentration of pyruvate is no more than 5 ppm.
141. The method of any one of the preceding Embodiments, wherein after contacting an aqueous initial preparation with the resin(s), the concentration of pyruvate is no more than 1 ppm.
142. The method of any one of the preceding Embodiments, wherein after contacting an aqueous initial preparation with the resin(s), the concentration of pyruvate is no more than 0.5 ppm.
143. The method of any one of the preceding Embodiments, wherein after contacting an aqueous initial preparation with the resin(s), the amount of pyruvate is reduced at about or at least about 90% compared to the aqueous initial preparation.
144. The method of any one of the preceding Embodiments, wherein after contacting an aqueous initial preparation with the resin(s), the amount of pyruvate is reduced at about or at least about 99% compared to the aqueous initial preparation.
145. The method of any one of the preceding Embodiments, wherein after contacting an aqueous initial preparation with the resin(s), the concentration of SO₄²⁻ is no more than 200 ppm.
146. The method of any one of the preceding Embodiments, wherein after contacting an aqueous initial preparation with the resin(s), the concentration of SO₄²⁻ is no more than 100 ppm.
147. The method of any one of the preceding Embodiments, wherein after contacting an aqueous initial preparation with the resin(s), the concentration of SO₄²⁻ is no more than 50 ppm.
148. The method of any one of the preceding Embodiments, wherein after contacting an aqueous initial preparation with the resin(s), the concentration of SO₄²⁻ is no more than 20 ppm.
149. The method of any one of the preceding Embodiments, wherein after contacting an aqueous initial preparation with the resin(s), the concentration of SO₄²⁻ is no more than 10 ppm.
150. The method of any one of the preceding Embodiments, wherein after contacting an aqueous initial preparation with the resin(s), the concentration of SO₄²⁻ is no more than 5 ppm.
151. The method of any one of the preceding Embodiments, wherein after contacting an aqueous initial preparation with the resin(s), the concentration of SO₄²⁻ is no more than 1 ppm.
152. The method of any one of the preceding Embodiments, wherein after contacting an aqueous initial preparation with the resin(s), the concentration of SO₄²⁻ is no more than 0.5 ppm.
153. The method of any one of the preceding Embodiments, wherein after contacting an aqueous initial preparation with the resin(s), the amount of SO₄²⁻ is reduced at about or at least about 90% compared to the aqueous initial preparation.
154. The method of any one of the preceding Embodiments, wherein after contacting an aqueous initial preparation with the resin(s), the amount of SO₄²⁻ is reduced at about or at least about 99% compared to the aqueous initial preparation.
155. The method of any one of the preceding Embodiments, wherein after contacting an aqueous initial preparation with the resin(s), the concentration of phosphate (H₂PO₄⁻, HPO₄²⁻, and/or PO₄³⁻) is no more than 200 ppm.
156. The method of any one of the preceding Embodiments, wherein after contacting an aqueous initial preparation with the resin(s), the concentration of phosphate (H₂PO₄⁻, HPO₄²⁻, and/or PO₄³⁻) is no more than 100 ppm.
157. The method of any one of the preceding Embodiments, wherein after contacting an aqueous initial preparation with the resin(s), the concentration of phosphate (H₂PO₄⁻, HPO₄²⁻, and/or PO₄³⁻) is no more than 50 ppm.
158. The method of any one of the preceding Embodiments, wherein after contacting an aqueous initial preparation with the resin(s), the concentration of phosphate (H₂PO₄⁻, HPO₄²⁻, and/or PO₄³⁻) is no more than 20 ppm.
159. The method of any one of the preceding Embodiments, wherein after contacting an aqueous initial preparation with the resin(s), the concentration of phosphate (H₂PO₄⁻, HPO₄²⁻, and/or PO₄³⁻) is no more than 10 ppm.
160. The method of any one of the preceding Embodiments, wherein after contacting an aqueous initial preparation with the resin(s), the concentration of phosphate (H₂PO₄⁻, HPO₄²⁻, and/or PO₄³⁻) is no more than 5 ppm.
161. The method of any one of the preceding Embodiments, wherein after contacting an aqueous initial preparation with the resin(s), the concentration of phosphate (H₂PO₄⁻, HPO₄²⁻, and/or PO₄³⁻) is no more than 1 ppm.
162. The method of any one of the preceding Embodiments, wherein after contacting an aqueous initial preparation with the resin(s), the concentration of phosphate (H₂PO₄⁻, HPO₄²⁻, and/or PO₄³⁻) is no more than 0.5 ppm.
163. The method of any one of the preceding Embodiments, wherein after contacting an aqueous initial preparation with the resin(s), the amount of phosphate (H₂PO₄⁻, HPO₄²⁻, and/or PO₄³⁻) is reduced at about or at least about 90% compared to the aqueous initial preparation.
164. The method of any one of the preceding Embodiments, wherein after contacting an aqueous initial preparation with the resin(s), the amount of phosphate (H₂PO₄⁻, HPO₄²⁻, and/or PO₄³⁻) is reduced at about or at least about 99% compared to the aqueous initial preparation.
165. The method of any one of the preceding Embodiments, comprising preparing the aqueous initial preparation from fermentation.
166. The method of any one of the preceding Embodiments, comprising biomass deactivation.
167. The method of any one of the preceding Embodiments, comprising preparing the aqueous initial preparation from fermentation, wherein the preparing comprises centrifugation.
168. The method of any one of the preceding Embodiments, comprising preparing the aqueous initial preparation from fermentation, wherein the preparing comprises batch centrifugation.
169. The method of any one of the preceding Embodiments, comprising preparing the aqueous initial preparation from fermentation, wherein the preparing comprises continuous centrifugation.
170. The method of any one of the preceding Embodiments, comprising preparing the aqueous initial preparation from fermentation, wherein the preparing comprises filtration.
171. The method of any one of the preceding Embodiments, comprising preparing the aqueous initial preparation from fermentation, wherein the preparing comprises microfiltration.
172. The method of any one of the preceding Embodiments, comprising preparing the aqueous initial preparation from fermentation, wherein the preparing comprises nanofiltration.
173. The method of any one of the preceding Embodiments, comprising a membrane filtration.
174. The method of any one of the preceding Embodiments, comprising a membrane filtration, wherein the pore size is about 10 μm or less.
175. The method of any one of the preceding Embodiments, comprising a membrane filtration, wherein the filter membrane shape is flat, a hollow tube fiber, tubular, spiral, or pleated.
176. The method of any one of the preceding Embodiments, comprising evaporating water from the preparation provided by contacting an aqueous initial preparation with an ion exchange resin.
177. The method of any one of the preceding Embodiments, comprising evaporating water from the preparation provided by contacting an aqueous initial preparation with an ion exchange resin under vacuum.
178. The method of any one of the preceding Embodiments, further comprising distillation that removes a component that has a lower boiling point than the glycol compound.
179. The method of any one of the preceding Embodiments, comprising distilling a glycol compound from a composition comprising the glycol compound.
180. The method of any one of Embodiments 178-179, wherein the distillation is performed under vacuum.
181. The method of any one of the preceding Embodiments, comprising polishing.
182. The method of any one of the preceding Embodiments, comprising polishing after evaporation and before distillation.
183. The method of any one of the preceding Embodiments, wherein polishing reduces levels of one or more salts.
184. The method of any one of the preceding Embodiments, wherein polishing comprises contact with a cation exchange resin.
185. The method of any one of the preceding Embodiments, wherein polishing comprises contact with an anion exchange resin.
186. The method of any one of the preceding Embodiments, wherein polishing comprises contact with a porous material.
187. The method of any one of the preceding Embodiments, wherein polishing comprises contact with activated carbon.
188. The method of any one of the preceding Embodiments, comprising decolorization of the glycol compound preparation.
189. The method of any one of the preceding Embodiments, wherein decolorization comprises hydrogenation.
190. The method of any one of the preceding Embodiments, wherein decolorization comprises contact with a porous material.
191. The method of any one of the preceding Embodiments, wherein decolorization comprises contact with activated carbon.
192. The method of any one of the preceding Embodiments, comprising deodorization of the glycol compound preparation.
193. The method of any one of the preceding Embodiments, wherein deodorization comprises hydrogenation.
194. The method of any one of the preceding Embodiments, wherein deodorization comprises contact with a porous material.
195. The method of any one of the preceding Embodiments, wherein deodorization comprises contact with activated carbon.
196. A glycol compound preparation prepared by a process comprising a method of any one of the preceding Embodiments.
197. A method of making a solvent, acrylate, polymer, polyurethane, polyester, adhesive, plasticizer, or coating comprising:
   providing a glycol compound preparation of Embodiment 197,
   converting the glycol compound into the solvent, acrylate, polymer, polyurethane, polyester, adhesive, plasticizer, or coating.
198. Use of a glycol compound preparation of Embodiment 197 in the manufacturing of a solvent, acrylate, polymer, polyurethane, polyester, adhesive, plasticizer, or coating.
199. A method of characterizing a resin as suitable for purifying an aqueous initial preparation comprising a glycol compound, wherein:
   the glycol compound has 5 to 12 carbon atoms, and
   wherein the aqueous initial preparation comprises greater than 50% water;
   the method comprising:
   determining whether the particle size, cross-linking density, functional group identity, functional group density, and hydrophilicity of the resin is within the appropriate or desired range or having the appropriate or desired identity.
200. A method of characterizing a resin as suitable for purifying an aqueous initial preparation comprising a glycol compound, comprising utilizing the resin in a method of any one of Embodiments 1-200 and assessing loss of a glycol compound during ion exchange purification.
201. The method of any one of the preceding Embodiments, wherein loss of a glycol compound during ion exchange is no more than about 5%.
202. The method of any one of the preceding Embodiments, wherein loss of a glycol compound during ion exchange is no more than about 4%.
203. The method of any one of the preceding Embodiments, wherein loss of a glycol compound during ion exchange is no more than about 3%.
204. The method of any one of the preceding Embodiments, wherein loss of a glycol compound during ion exchange is no more than about 2%.
205. The method of any one of the preceding Embodiments, wherein loss of a glycol compound during ion exchange is no more than about 1%.
206. The method of any one of Embodiments 201-205, wherein the resin is determined to be suitable.
207. The method of any one of the preceding Embodiments, wherein loss of a glycol compound without pre-masking and pre-recovering is about or at least about 1.5, 2, 3, 4, 5, 6, 7, 8, 9, or 10 fold of the loss without pre-masking and without pre-recovering.
208. The method of any one of the preceding Embodiments, wherein loss of a glycol compound without pre-masking and pre-recovering is about or at least about 2 fold of the loss without pre-masking and without pre-recovering.
209. The method of any one of the preceding Embodiments, wherein loss of a glycol compound without pre-masking and pre-recovering is about or at least about 5 fold of the loss without pre-masking and without pre-recovering.
210. The method of any one of the preceding Embodiments, wherein loss of a glycol compound without pre-masking and pre-recovering is about or at least about 10 fold of the loss without pre-masking and without pre-recovering.
211. A method of characterizing a resin as suitable for purifying an aqueous initial preparation comprising a glycol compound, wherein:
   the glycol compound has 5 to 12 carbon atoms, and
   wherein the aqueous initial preparation comprises greater than 50% water; the method comprising:
   contacting the resin with the aqueous initial preparation;
   assessing glycol compound retention on the resin;
   determining glycol compound retention is reduced relative to that observed under otherwise comparable conditions with a hydrophobic resin.
212. A purified preparation of a glycol compound in which the glycol compound is present in an amount that is about at least about 50%-100%, more than, or about or at least about or about 2, 3, 4, 5, 6, 7, 8, 9, 10, 11, 12, 13, 14, 15, 16, 17, 18, 19, 20, 30, 40, 50 or 100 fold of, that present in an aqueous initial preparation from which the glycol compound is purified.
213. A purified preparation of a glycol compound in which pyruvate is present in an amount that is at least 10-fold less than that present in the aqueous preparation from which the glycol compound is purified.
214. A purified preparation of a glycol compound in which phosphate is present in an amount that is at least 10-fold less than that present in an aqueous initial preparation from which the glycol compound is purified.
215. A purified preparation of a glycol compound in which sulfate is present in an amount that is at least 10-fold less than that present in an aqueous initial preparation from which the glycol compound is purified.
216. A purified preparation of a glycol compound in which potassium is present in an amount that is at least 10-fold less than that present in an aqueous initial preparation from which the glycol compound is purified.
217. A purified preparation of a glycol compound in which magnesium is present in an amount that is at least 10-fold less than that present in an aqueous initial preparation from which the glycol compound is purified.
218. The method or preparation of any one of the preceding Embodiments, wherein the glycol compound has about 5 to 12 carbon atoms.
219. The method or preparation of any one of Embodiments 1-218, wherein the glycol compound has the structure of L(OH)ₙ wherein L is a C₅₋₁₂ hydrocarbon moiety and n is 2 or 3.
220. The method or preparation of any one of Embodiments 1-218, wherein the glycol compound has the structure of L(OH)ₙ wherein L is a C₅₋₈ hydrocarbon moiety and n is 2 or 3.
221. The method or preparation of any one of the preceding Embodiments, wherein n is 2.
222. The method or preparation of any one of the preceding Embodiments, wherein L is linear.
223. The method or preparation of any one of Embodiments 1-221, wherein L is branched.
224. The method or preparation of any one of the preceding Embodiments, wherein L is saturated.
225. The method or preparation of any one of Embodiments 1-223, wherein L is partially unsaturated.
226. The method or preparation of any one of Embodiments 1-219, wherein the glycol compound has the structure of HO(CH₂)ₘOH, wherein m is 5-12.
227. The method or preparation of any one of Embodiments 1-219, wherein the glycol compound has the structure of HO(CH₂)ₘOH, wherein m is 5-8.
228. The method or preparation of any one of Embodiments 1-219, wherein the glycol compound is HO(CH₂)₅OH.
229. The method or preparation of any one of Embodiments 1-219, wherein the glycol compound is HO(CH₂)₆OH.
230. The method or preparation of any one of Embodiments 1-219, wherein the glycol compound is HO(CH₂)₇OH.
231. The method or preparation of any one of Embodiments 1-219, wherein the glycol compound is HO(CH₂)₈OH.
232. The method or preparation of any one of Embodiments 1-219, wherein the glycol compound is HO(CH₂)₉OH.
233. The method or preparation of any one of Embodiments 1-219, wherein the glycol compound is HO(CH₂)₁₀OH.
234. The method or preparation of any one of Embodiments 1-219, wherein the glycol compound is HO(CH₂)₁₁OH.
235. The method or preparation of any one of Embodiments 1-219, wherein the glycol compound is HO(CH₂)₁₂OH.

### EXEMPLIFICATION

### Example 1: Exemplary Filtration Processes

A step in the purification of a chemical, in this case a glycol compound, from a fermentation broth may begin with centrifugation and/or filtration to physically exclude insoluble material and large molecules. Decreasing the pore size for filtration further increases the purity of the permeate, but remaining chemical constituents in the aqueous preparations that are not separable by size. Electrostatic properties amplified by pH modulation are used in the task of separating cations and anions from each other. Certain ionic compounds may be preferentially bound to an insoluble support structure (e.g., an ion exchange resin).

For example, in some embodiments, it may be desirable to perform one or more filtration steps, particularly where a source preparation contains one or more materials amenable to size separation relative to a glycol compound(s) of interest.

In particular embodiments (including specifically in certain embodiments in which a relevant glycol compound was produced by fermentation such that an aqueous preparation includes cells or cell components), one or more micro- and/or nano-filtration steps may desirably be performed.

The present Example describes certain steps in a provided method for purifying a glycol compound from fermentation broth containing it.

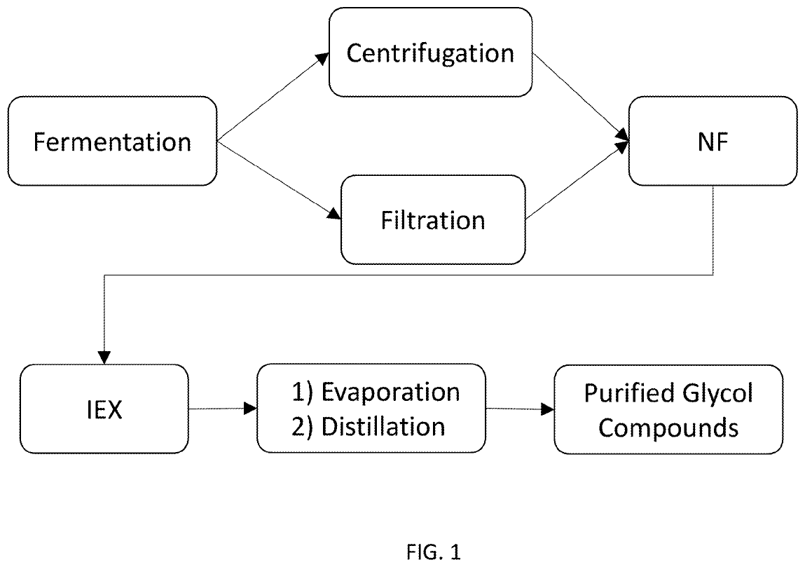

The overall protocol utilized certain embodiments is graphically represented in FIG. 1, which shows an aqueous preparation (specifically, a fermentation broth) containing a glycol compound, e.g., a higher-order fatty alcohol compound (specifically, 1,6-hexanediol (HDO) in this case) was first treated with tangential flow filtration (TFF) and/or centrifugation. In some embodiments, two stages of TFF were used: microfiltration (MF) and nanofiltration (NF). Each of these filtrations used a polymeric membrane to separate the target molecule (HDO) from impurities in the fermentation broth via size separation.

Fermentation broth was generated by growing a strain of E. coli in media containing a phosphate buffer, metal salts, amino acids, and a carbon source, glycerol. Cells were grown in a bioreactor (e.g., in some embodiments at commercial scale such as 10 kL or more as described herein) until they reached an optical density (OD) over 100. Whole broth was then diluted with deionized water such that the optical density of cells was 50. Commercial 1,6-hexanediol was added to the broth at a concentration of 20 g/L or 100 g/L to provide a sample for testing.

Insoluble matter of the fermentation broth was removed by tangential flow filtration (TFF). Fermentation broth was subjected to microfiltration (0.1 μm PVDF, 800 kDa MWCO PVDF (polyvinylidene difluoride)) followed by nanofiltration (150-300 Da PA, 300-500 Da PA (polyamide)).

Microfiltration, as depicted, was used to retain the suspended cell mass and most of the cell debris while allowing the rest of the material to permeate. Microfiltration can encompass a range of membrane pore sizes or molecular weight cutoffs (MWCO)), e.g., from 100-800 kDa. Typically, microfiltration is intended to reduce the amount of solid cell mass (quantified by optical density, packed cell volume, or dry cell weight) while not retaining any of the HDO (quantified by HPLC). Particularly for larger-scale isolations, it may be desirable to maximize membrane flux of such a microfiltration step.

Nanofiltration, as depicted, was used to permeate HDO and other smaller molecules (metal ions, monomeric sugars, small organic acids, etc.), while retaining larger molecules (small cell debris, proteins, dimeric and larger sugars, large organic acids, etc.). Nanofiltration also describes a range of MWCOs, ranging from 200 Da to 3 kDa. Typically, nanofiltration is intended to reduce impurities (e.g., as may be quantified by HPLC and/or conductivity) while not retaining HDO (e.g., as may be quantified by HPLC). Particularly for larger-scale isolations, it may be desirable to maximize membrane flux of such a nanofiltration step.

Microfiltration and nanofiltration were tested on two membranes, respectively:
Microfiltration: 0.1 μm PVDF, 800 kDa MWCO PVDF (polyvinylidene difluoride)
Nanofiltration: 150-300 Da PA, 300-500 Da PA (polyamide; can also be of other materials such as polysulfone, cellulose, etc.).

The primary metrics utilized to evaluate isolation efficiency included: (1) optical density reduction, (2) rejection of impurities, and (3) rejection of HDO (1,6-hexane glycol). Rejection was used in place of yield in this case because yield considers losses due to the particularities of the system and operation (e.g., holdup volume, losses during loading/unloading, dilution, and losses due to operator error). The present disclosure provides an insight that rejection can give a more accurate picture of how HDO interacts with a membrane. For both microfiltration and nanofiltration, zero rejection is the theoretical ideal, as rejection of HDO contributes to yield losses. Rejection is defined as:

$$Rejection ( \% ) = 100 \% * ( 1 - \frac{Concentration of Permeate}{Concentration of Feed} )$$

1,6-Hexanediol rejection on various membranes demonstrates efficiency of the filtration process by retaining insoluble matter.

TABLE 1
At consistent operating conditions, 1,6-hexanediol
rejection was low for all membranes.

| | Feed Conc. | Perm Conc. | Rejection |
|---|---|---|---|
| | (g/L) | (g/L) | (%) |
| 0.1 μm Microfiltration | 18.21 | 17.98 | 1.3% |
| 800 kDa Microfiltration | 19.79 | 19.17 | 3.13% |
| 150-300 Da Nanofiltration | 16.28 | 15.64 | 3.93% |
| 200-400 Da Nanofiltration | 17.98 | 17.95 | 0.17% |

The 0.1 μm filter allowed a small amount of suspended solids to enter the permeate stream, while the 800 kDa filter removed effectively all of the suspended solids.

TABLE 2
Optical density (OD) reduction on microfiltration.

| Microfiltration pore | | Feed OD | Permeate OD | Rejection (%) |
|---|---|---|---|---|
| 0.1 | μm | 50 | 1.1 | 97.8 |
| 800 | kDa | 50 | 0.10 | 99.8 |

In some embodiments, microfiltration membranes did not reduce conductivity. In some embodiments, nanofiltration membranes reduced conductivity (μS/cm), in some cases, by about 65%.

TABLE 3
Conductivity Reduction (all values in
μS/cm unless otherwise indicated).

| | Feed | | | Conductivity |
|---|---|---|---|---|
| | Solution | Permeate | Retentate | Reduction (%) |
| 0.1 μm Microfiltration | 6615 | 6687 | 6900 | 0% |
| 800 kDa | 9648 | 9170 | 8694 | ~0% |
| Microfiltration | | | | (accounting |
| | | | | for dilution) |
| 300-500 Da | 8694 | 2909 | 17630 | 66.5% |
| Nanofiltration | | | | |
| 150-300 Da | 3365 | 1220 | 8478 | 63.7% |
| Nanofiltration | | | | |

In some embodiments, a 0.1 μm membrane has higher flux across a range of transmembrane pressures compared to a 800 kDa membrane.

TABLE 4
TMP and Flux for microfiltration - 0.1 μm and 800 kDa.

| | 0.1 μm MF Flux | 800 kDa MF Flux |
|---|---|---|
| TMP (psi) | (L/m²/h) | (L/m²/h) |
| 40 | 23.2 | 14.8 |
| 50 | 28.8 | 16 |
| 60 | 30.4 | 16.8 |

In some embodiments, a 150-300 Da membrane has higher flux across a range of transmembrane pressures compared to a 300-500 membrane.

TABLE 5
TMP and Flux for nanofiltration - 150-300 Da and 300-500 Da

| | 300-500 Da Flux | 150-300 Da Flux |
|---|---|---|
| TMP (psi) | (L/m²/h) | (L/m²/h) |
| 100 | 5.65 | 8.63 |
| 150 | 12.26 | 19.13 |
| 200 | 17.42 | 25.86 |
| 250 | 21.29 | 34.5 |
| 270 | 23.87 | 37.5 |

Operating conditions were varied to evaluate permeate flux. Transmembrane pressures between 35 psi and 80 psi were applied for microfiltration. Transmembrane pressures between 100 psi and 350 psi were applied for nanofiltration. Transmembrane pressure (TMP) is a pressure reading that takes into account the pressure drop across the membrane as well as across the membrane, and is here defined as:

$$TMP ( psi ) = \frac{{P}_{inlet} + {P}_{outlet}}{2} - {P}_{permeate}$$

Permeate flux is presented as permeate flow rate normalized by membrane area. In some embodiments, microfiltration membranes have a surface area of 0.15 square meters, and nanofiltration membranes have a surface area of 0.35 square meters. All conditions were run at total reflux (retentate was continually fed to the feed tank). The present Example demonstrates, for example, that:
(1) assessment of “rejection” level (i.e., of the amount of material of interest that does not pass through a filter) is a particularly useful parameter when evaluating and/or utilizing filtration technologies (e.g., in the context of glycol compound isolation as described herein, e.g., of long chain glycol compounds, and in particular in aqueous preparations, etc).
(2) in some embodiments of useful technologies, 0.1 μm microfiltration need not be utilized;
(3) in some embodiments of useful technologies, nanofiltration may be more useful than microfiltration;
(4) in some embodiments, 150-300 Da membrane filtration may be particularly useful.

More particularly, the present Example demonstrates that either set of membranes could be viable for glycol compound (e.g., HDO) isolation (e.g., as initial isolation steps applied to an aqueous preparation such as an aqueous fermentate). Specifically, the results demonstrated that a glycol compound such as HDO was not rejected to a significant degree on (i.e., mostly passed through) any of the four membranes considered.

The present results further established that solids were sufficiently or fully removed (e.g., as determined by HPLC and/or OD 600 nm and/or 660 nm), by the 800 kDa microfilter with only a modest reduction in flux; the present disclosure teaches that, in certain embodiments, an 800 kDa microfilter may desirably be utilized (e.g., particularly for processing of fermentate preparation(s) or other preparations derived therefrom). The present disclosure furthermore teaches that use of a 150-300 Da filter in nanofiltration can achieve higher flux and comparable conductivity reduction relative to other filters; the present disclosure teaches that, in certain embodiments, a 150-300 Da filter is used for nanofiltration (e.g., particularly for processing of fermentate preparation(s) or other preparations derived therefrom).

In some embodiments, alternative or additional cell removal methods can be utilized, such as dead end filtration (filter press, leaf press, etc), centrifugation (DSC, decanter, etc). In some embodiments, continuous centrifugation can be used. In some embodiments, continuous centrifugation is cage centrifugation. In some embodiments, continuous centrifugation is disc centrifugation. In some embodiments, continuous centrifugation is nozzle centrifugation. Other initial isolation methods could also be feasible, such as solvent extraction or fixed bed adsorption.

### Example 2: Exemplary Ion Exchange Technologies

The present Example documents certain surprising insights provided by the present disclosure:
(i) that exposing a chromatography resin (e.g., an ion exchange resin) to a glycol compound prior to utilizing the resin to isolate that compound from a sample (e.g., from an aqueous sample, e.g., an aqueous initial preparation as described herein) improves purification efficiency achieved by the resin. That is, more glycol compound passes through a resin that has been pre-treated as described herein than does through an otherwise comparable resin that has not been so pre-treated; and
(ii) that exposing a chromatography resin on which a glycol compound as described herein (e.g., a long chain glycol compound from an aqueous preparation) has been retained to wash with a solvent system, for example, water, an alcohol (e.g., a short chain alcohol such as methanol and ethanol), an appropriate organic solvent (e.g., THF, and acetone) or a mixture of two or more of such solvents, can usefully remove glycol compound material(s) from the resin and in some embodiments to a greater extent than it removes non-glycol compound.

In some embodiments, the following processes were performed:
(i) “IEX-I” (reference process; “normal, comparative example”)
(a) 40 mL preconditioned resin was stirred and processed with 200 mL simulated solution for 1 hour. The resin was removed by filtration, and the processed filtrate was analyzed by HPLC. HDO loss was assessed.
(ii) “IEX-II” (pre-masking+post-recovering)
(a) 40 mL preconditioned resin was stirred and processed with aqueous 0.5% HDO (150 g) for 30 minutes (pre-masking), and the resin was recovered by filtration The filtrate was analyzed by HPLC. HDO loss was assessed.
(b) The recovered resin was stirred and processed with 200 mL of simulated solution for 1 hour. The resin was recovered by filtration, and the filtrate was analyzed by HPLC. HDO loss was assessed.
(c) The recovered resin was stirred and processed with water (200 mL) for 1 hour (post-recovering). The resin was removed by filtration, and the filtrate was analyzed by HPLC. HDO recovery was assessed.
(iii) “IEX-III” (pre-masking only)
(a) 40 mL preconditioned resin was stirred and processed with aqueous 0.5% HDO (150 g) for 30 minutes (pre-masking), and the resin was recovered by filtration. The filtrate was analyzed by HPLC. HDO loss was assessed.
(b) The recovered resin was stirred and processed with 200 mL of simulated solution for 1 hour. The resin was recovered by filtration, and the filtrate was analyzed by HPLC. HDO loss was assessed.
(iv) “IEX-IV” (post-recovering)
(a) 40 mL preconditioned resin was stirred and processed with 200 mL of simulated solution for 1 hour. The resin was recovered by filtration, and the processed filtrate was analyzed by HPLC. HDO loss was assessed.
(b) The recovered resin was stirred and processed with water (200 g) for 1 hour (post-recovering). The resin was removed by filtration, and the filtrate was analyzed by HOD. HDO recovery was assessed.

Glycol-compound loss analysis and quantification. Each sampling solution was analyzed by HPLC and IC.

$$ratio of total glycol loss = 1 - ( \frac{final glycol concentration}{initial glycol concentration} )$$

In some embodiments, ratio of total HDO loss was calculated as (can also be utilized for other glycol compounds):

$$Ratio of total HDO loss = \frac{Total HDO loss amount of IEX process}{HDO amount of feed solution} [ wt \% ]$$

wherein Total HDO loss amount of IEX process=HDO loss amount by IEX+HDO loss amount by pre-masking (if pre-masking is performed)−HDO recovered amount by post-recovering (if post-masking is performed).

Simulated Solution A was devised to emulate the fermentation following microfiltration and nanofiltration. Phosphate, sulfate, and pyruvate serve as an ensemble of undesired anions, and potassium and magnesium serve as an ensemble of undesired cations, to be purified from an aqueous preparation of 1,6-hexanediol.

TABLE 6
Simulated Solution A.

| Compound | Concentration /wt % |
|---|---|
| 1,6-hexanediol (HDO) | 1.50 |
| K₃PO₄ | 0.75 |
| MgSO₄ | 0.05 |
| pyruvic acid | 0.35 |
| Ion exchanged water | — |

This example demonstrates the effectiveness of pre-masking and/or post-recovering on minimizing loss of an isolation target when various ion exchange resins were utilized. Unless otherwise noted, concentrations after IEX are typically for IEX flow-through, and ppm is w/w.

TABLE 7
Certain results for a strong cation exchange
resin, polystyrene (SK1BH) Mitsubishi DIAION.

| | Ratio of total HDO loss/wt % | K⁺/ppm | Mg²⁺/ppm |
|---|---|---|---|
| Simulated | — | 3100 | 91 |
| Solution A | | | |
| IEX-I | 11.1 | 140 | 0.8 |
| IEX-II | 0.635 | 143 | 1.0 |
| IEX-III | 8.47 | 151 | 1.2 |
| IEX-IV | 1.14 | 135 | 0.9 |

TABLE 8
Certain results for a strong anion exchange resin,
polystyrene (SA10AOH) Mitsubishi DIAION.

| | Ratio of total | Pyruvate/ | SO₄²⁻/ | PO₄³⁻/ |
|---|---|---|---|---|
| | HDO loss/wt % | ppm | ppm | ppm |
| Simulated | — | 3200 | 350 | 2400 |
| Solution A | | | | |
| IEX-I | 13.4 | 53 | 25 | 0 |
| IEX-II | 4.07 | 51 | 21 | 0 |
| IEX-III | 11.7 | 53 | 20 | 0 |
| IEX-IV | 6.33 | 61 | 23 | 0 |

In some embodiments, it was observed that weak, hydrophobic anion exchange resins do not perform as well as other ion exchange resins.

TABLE 9
Certain results for a weak anion exchange resin,
polystyrene (WA21J) Mitsubishi DIAION.

| | Ratio of total | Pyruvate/ | SO₄²⁻/ | PO₄³⁻/ |
|---|---|---|---|---|
| | HDO loss/wt % | ppm | ppm | ppm |
| Simulated | — | 3200 | 350 | 2400 |
| Solution A | | | | |
| IEX-I | 13.4 | 150 | 8.4 | 4.6 |
| IEX-II | 9.44 | 142 | 8.1 | 4.0 |
| IEX-III | 13.5 | 147 | 8.1 | 4.9 |
| IEX-IV | 6.33 | 152 | 8.2 | 4.8 |

This example demonstrates that a hydrophilic anion exchange resin can be extremely effective in adsorbing undesired anions from an aqueous initial preparation, while minimizing the loss of the isolation target. In some embodiments, a hydrophilic anion exchange resin adsorbs little of the desired isolation target from an aqueous initial preparation and then preferentially released, substantially free of inorganic anionic contaminants. In some embodiments, losses are cut by more than 5000 when the operation is performed with post-recovery washes.

TABLE 10
Certain results for a strong anion exchange resin,
polyacrylic (HPR4850 Cl) AmberLite DuPont.

| | Ratio of total | Pyruvate/ | SO₄²⁻/ | PO₄³⁻/ |
|---|---|---|---|---|
| | HDO loss/wt % | ppm | ppm | ppm |
| Simulated | — | 3200 | 350 | 2400 |
| Solution A | | | | |
| IEX-I | 0.693 | 100 | 4.4 | 1.8 |
| IEX-II | 0.341 | 110 | 4.7 | 1.1 |
| IEX-III | 0.684 | 103 | 4.7 | 1.5 |
| IEX-IV | 0.338 | 102 | 4.7 | 1.8 |

TABLE 11
Certain results for a strong anion exchange resin,
polyacrylic (SCAV4 Cl) AmberLite DuPont.

| | Ratio of total | Pyruvate/ | SO₄²⁻/ | PO₄³⁻/ |
|---|---|---|---|---|
| | HDO loss/wt % | ppm | ppm | ppm |
| Simulated | — | 3200 | 350 | 2400 |
| Solution A | | | | |
| IEX-I | 0.664 | 180 | 6.4 | 3.6 |
| IEX-II | 0.289 | 178 | 6.1 | 3.1 |
| IEX-III | 0.661 | 183 | 6.8 | 3.5 |
| IEX-IV | 0.297 | 177 | 7.1 | 3.2 |

This example demonstrates that hydrophilic polyacrylic resin, when treated with appropriate pre-masking and/or post-recovering has a greatly improved performance while selectively removing undesired cationic contaminants.

TABLE 12
Certain results for a weak cation exchange resin,
polyacrylic (WK60L) Mitsubishi Relite.

| | Ratio of total HDO loss/wt % | K⁺/ppm | Mg²⁺/ppm |
|---|---|---|---|
| Simulated | — | 3100 | 91 |
| Solution A | | | |
| IEX-I | 5.92 | 210 | 1.2 |
| IEX-II | 0.01 | 225 | 1.5 |
| IEX-III | 2.24 | 241 | 2.1 |
| IEX-IV | 0.22 | 203 | 1.8 |

This example demonstrates that pre-masking a hydrophilic resin decreases the adsorptive loss of a glycol compound isolation target while still effectively removing undesired anions.

TABLE 13
Certain results for a weak anion exchange resin,
polyacrylic (IRA67) AmberLite DuPont.

| | Ratio of total | Pyruvate/ | SO₄²⁻/ | PO₄³⁻/ |
|---|---|---|---|---|
| | HDO loss/wt % | ppm | ppm | ppm |
| Simulated | — | 3200 | 350 | 2400 |
| Solution A | | | | |
| IEX-I | 2.18 | 240 | 15.8 | 7.9 |
| IEX-II | 1.13 | 251 | 13.7 | 8.0 |
| IEX-III | 1.91 | 248 | 14.2 | 7.4 |
| IEX-IV | 1.57 | 257 | 14.9 | 7.6 |

This example demonstrates that loss from IEX with a less hydrophilic resin is diminished by the pre-masking and/or post-recovering treatment(s) of the resin.

TABLE 14
Certain results for a weak cation exchange resin,
polymethacrylic (WK10) Mitsubishi DIAION.

| | Ratio of total HDO loss/wt % | K⁺/ppm | Mg²⁺/ppm |
|---|---|---|---|
| Simulated | — | 3100 | 91 |
| Solution A | | | |
| IEX-I | 3.28 | 219 | 1.9 |
| IEX-II | 0.25 | 243 | 3.4 |
| IEX-III | 1.71 | 238 | 3.7 |
| IEX-IV | 0.71 | 214 | 2.6 |

Advantages and benefits of the provided technologies are also demonstrated using other glycol compounds.

TABLE 15
Simulated Solution B.

| Compound | Concentration (wt %) |
|---|---|
| 1,4-butanediol (BDO) | 1.50 |
| K₃PO₄ | 0.75 |
| MgSO₄ | 0.05 |
| pyruvic acid | 0.35 |
| Ion exchanged water | — |

This example demonstrates the pre-masking and/or post-recovering can reduce loss of BDO.

TABLE 16
Certain results for a strong cation exchange
resin, polystyrene (SK1BH) Mitsubishi DIAION.

| | Ratio of total BDO Loss/wt % | K⁺/ppm | Mg²⁺/ppm |
|---|---|---|---|
| Simulated | — | 3100 | 91 |
| Solution B | | | |
| IEX-I | 1.05 | 134 | 1.8 |
| IEX-II | 0.335 | 136 | 1.3 |
| IEX-III | 0.870 | 145 | 1.9 |
| IEX-IV | 0.531 | 130 | 0.9 |

TABLE 17
Simulated Solution C.

| Compound | Concentration (wt %) |
|---|---|
| 1,8-octanediol (ODO) | 1.50 |
| K₃PO₄ | 0.75 |
| MgSO₄ | 0.05 |
| pyruvic acid | 0.35 |
| Ion exchanged water | — |

This example demonstrates the pre-masking and/or post-recovering can reduce loss of ODO. In some embodiments, as the carbon chain increases, loss of glycol compounds and/or adsorptive capacity of resins increase. As demonstrated herein, pre-masking and/or post-recovery can greatly diminish loss of a desired glycol.

TABLE 18
Certain results for a strong cation exchange
resin, polystyrene (SK1BH) Mitsubishi DIAION.

| | Ratio of total ODO Loss/wt % | K⁺/ppm | Mg²⁺/ppm |
|---|---|---|---|
| Simulated | — | 3100 | 91 |
| Solution C | | | |
| IEX-I | 45.1 | 143 | 1.4 |
| IEX-II | 20.2 | 137 | 1.1 |
| IEX-III | 38.5 | 150 | 1.7 |
| IEX-IV | 24.8 | 131 | 1.7 |

This example demonstrates that a resin may have preferential adsorption of longer alkyl glycol compounds. In some embodiments, loss of longer chain glycol compounds is higher than shorter chain glycol compounds, particularly without pre-masking and/or post-recovering.

TABLE 19
Simulated Solution D (a mixture of many glycol compounds).

| Glycol compound | Concentration (wt %) |
|---|---|
| 1,3-propanediol | 0.10 |
| 1,4-butanediol | 0.10 |
| 1,5-pentanediol | 0.10 |
| 1,6-hexandiol | 0.10 |
| 1,7-heptanediol | 0.10 |
| 1,8-octanediol | 0.10 |
| Ion-exchanged water | — |

TABLE 20
Certain results for a strong cation resin, polystyrene
(SK1BH) Mitsubishi DIAION using IEX-I.

| Glycol compound | Ratio of each total diol loss/wt % |
|---|---|
| 1,3-propanediol | 0.105 |
| 1,4-butanediol | 1.31 |
| 1,5-pentanediol | 4.95 |
| 1,6-hexanediol | 12.5 |
| 1,7-heptanediol | 21.6 |
| 1,8-octanediol | 42.3 |

TABLE 21
Certain results for a strong anion resin, polystyrene
(SA10AOH) Mitsubishi DIAION using IEX-I.

| Glycol compound | Ratio of each total diol loss/wt % |
|---|---|
| 1,3-propanediol | 0.112 |
| 1,4-butanediol | 1.19 |
| 1,5-pentanediol | 5.02 |
| 1,6-hexanediol | 12.8 |
| 1,7-heptanediol | 21.5 |
| 1,8-octanediol | 40.3 |

This example demonstrates that ion exchange resin of certain support structure, e.g., hydrophilic polymers such as polyacrylic gel, can greatly enhance isolation of a glycol compound from an aqueous initial preparation.

TABLE 22
Certain results for a strong anion resin,
polyacrylic, gel (HPR4850 Cl) using IEX-I.

| Glycol compounds | Ratio of each total diol loss/wt % |
|---|---|
| 1,3-propanediol | 0.306 |
| 1,4-butanediol | 0.693 |
| 1,5-pentanediol | 0.583 |
| 1,6-hexanediol | 0.280 |
| 1,7-heptanediol | 0.424 |
| 1,8-octanediol | 0.609 |

TABLE 23
Certain results for a strong anion resin, polyacrylic,
macroporous (SCAV4 Cl) using IEX-I.

| Glycol compounds | Ratio of each total diol loss/ wt % |
|---|---|
| 1,3-propanediol | 0.953 |
| 1,4-butanediol | 0.664 |
| 1,5-pentanediol | 0.584 |
| 1,6-hexanediol | 0.607 |
| 1,7-heptanediol | 1.200 |
| 1,8-octanediol | 0.958 |

### Example 3: Exemplary Ion Exchange Technologies

Among other things, the present disclosure demonstrates that various provided technologies can be scaled up for large scale purification and production, including various commercial scales. Certain useful technologies are described below as examples.

Resin pre-conditioning. Pre-conditioning of resin can be performed using various technologies. Certain useful technologies are described below as examples. Various parameters, e.g., resin integrity and effluent pH, may be assessed before, during, and/or conditioning.

Pre-conditioning of ion exchange resin (H/OH or free type resin): Commercial resin (400 mL) was stirred and washed with water (3 kg) for 1 hour. The resin was soaked in pure water for 12 hours. The soaked resin was recovered by filtration.

Pre-conditioning of ion exchange resin (Cl-type resin): Resin (400 mL) was stirred and regenerated to the OH-type in aqueous 10% NaOH (2 kg). The regenerated resin was stirred and washed with water (3 kg). The washed resin was soaked in water for 12 hours. The soaked resin was recovered by filtration.

As described herein, various technologies comprise pre-masking and/or post-recovering. For pre-masking, various parameters, e.g., glycol compound concentrations and/or amounts and mask time, may be assessed to identify suitable conditions in accordance with the present disclosure. Those skilled in the art reading the present disclosure can assess and identify suitable parameters, e.g., solvent systems and treatment time, for post-recovering in accordance with the present disclosure. Certain pre-masking and/or post-recovering technologies are described in one or more protocols below as examples.

### Protocol IEX-I

(i) Pre-conditioned resin (400 mL) was stirred in contact with simulated solution (2 L) for 1 hour. The resin was removed by filtration, and the processed filtrate was analyzed (e.g., by HPLC and/or IC).

### Protocol IEX-II (Pre-Mask and Post-Recovering)

(i) Pre-masking: Pre-conditioned resin (400 mL) was stirred in contact with aqueous 0.5% glycol compound, e.g., HDO, (1.5 kg) for 30 minutes. The resin was recovered by filtration. The filtrate was analyzed (e.g., by HPLC and/or IC). Loss of a glycol compound, e.g., HDO, during pre-masking was assessed.
(ii) The resin was stirred in contact with a simulated solution (2 L) for 1 hour. The resin was then recovered by filtration and the filtrate was analyzed (e.g., by HPLC and/or IC). Loss of a glycol compound, e.g., HDO, was assessed.
(iii) Post-recovering: The recovered resin was stirred with water (2 L) for 1 hour. The resin was then recovered by filtration and the filtrate was analyzed (e.g., by HPLC and/or IC). Recovery of a glycol compound, e.g., HDO, was assessed.

### Protocol IEX-III (Pre-Masking)

(i) Pre-masking: Pre-conditioned resin (400 mL) was stirred in contact with aqueous 0.5% glycol compound, e.g., HDO, (1.5 kg) for 30 minutes. The resin was recovered by filtration. The filtrate was analyzed (e.g., by HPLC and/or IC). Loss of a glycol compound, e.g., HDO, during pre-masking was assessed.
(ii) The resin was stirred in contact with a simulated solution (2 L) for 1 hour. The resin was then recovered by filtration and the filtrate was analyzed (e.g., by HPLC and/or IC). Loss of a glycol compound, e.g., HDO, was assessed.

### Protocol IEX-IV (Post-Recovering)

(i) Pre-conditioned resin (400 mL) was stirred in contact with simulated solution (2 L) for 1 hour. The resin was then recovered by filtration and the filtrate was analyzed (e.g., by HPLC and/or IC). Loss of a glycol compound, e.g., HDO, was assessed.
(ii) Post-recovering: The recovered resin was stirred with water (2 L) for 1 hour. The resin was then recovered by filtration and the filtrate was analyzed (e.g., by HPLC and/or IC). Recovery of a glycol compound, e.g., HDO, was assessed.

Certain large-scale results using Simulated Solution A and the protocols I-IV are listed below. As demonstrated herein, in various embodiments the present disclosure provides technologies that can effectively remove anions and/or cations and reduce glycol compound loss. In some embodiments, for resin with hydrophobic base polymers (e.g., polystyrene), pre-masking and/or post-recovering can dramatically reduce glycol compound loss. In some embodiments, provided technologies comprise utilization of resin with hydrophilic base polymers. In some embodiments, provided technologies comprise utilization of resin with polyacrylic polymers. In some embodiments, as demonstrated herein polyacrylic polymers can provide effective ion exchange and low levels of glycol compound loss.

TABLE 25
Certain results for a strong cation exchange
resin, polystyrene (SK1BH) Mitsubishi DIAION.

| | Ratio of total HDO loss/wt % | K⁺/ppm | Mg²⁺/ppm |
|---|---|---|---|
| Simulated | — | 3100 | 91 |
| Solution A | | | |
| IEX-I | 10.3 | 120 | 0.4 |
| IEX-II | 0.431 | 123 | 0.7 |
| IEX-III | 8.13 | 131 | 1.0 |
| IEX-IV | 1.01 | 130 | 0.4 |

TABLE 26
Certain results for a strong anion exchange resin,
polystyrene (SA10AOH) Mitsubishi DIAION.

| | Ratio of total | | | |
|---|---|---|---|---|
| | HDO loss/wt % | Pyruvate/ppm | SO₄²⁻/ppm | PO₄³⁻/ppm |
| Simulated | — | 3200 | 350 | 2400 |
| Solution A | | | | |
| IEX-I | 13.0 | 49 | 19 | 0 |
| IEX-II | 4.01 | 57 | 19 | 0 |
| IEX-III | 10.9 | 51 | 22 | 0 |
| IEX-IV | 5.87 | 51 | 13 | 0 |

TABLE 27
Certain results for a strong anion exchange resin,
polyacrylic (HPR4850 Cl) AmberLite DuPont.

| | Ratio of total | | | |
|---|---|---|---|---|
| | HDO loss/wt % | Pyruvate/ppm | SO₄²⁻/ppm | PO₄³⁻/ppm |
| Simulated | — | 3200 | 350 | 2400 |
| Solution A | | | | |
| IEX-I | 0.680 | 90 | 4.3 | 1.6 |
| IEX-II | 0.329 | 86 | 4.4 | 1.1 |
| IEX-III | 0.673 | 97 | 4.1 | 1.2 |
| IEX-IV | 0.331 | 98 | 4.0 | 1.5 |

TABLE 28
Certain results for a strong anion exchange resin,
polyacrylic (SCAV4 Cl) AmberLite DuPont.

| | Ratio of total | | | |
|---|---|---|---|---|
| | HDO loss/wt % | Pyruvate/ppm | SO₄²⁻/ppm | PO₄³⁻/ppm |
| Simulated | — | 3200 | 350 | 2400 |
| Solution A | | | | |
| IEX-I | 0.659 | 175 | 6.4 | 3.5 |
| IEX-II | 0.281 | 173 | 6.2 | 3.0 |
| IEX-III | 0.624 | 174 | 6.9 | 3.4 |
| IEX-IV | 0.243 | 170 | 6.1 | 3.1 |

As described herein, various solvent systems, e.g., those that are or comprise an alcohol such as ethanol, can be utilized for post-recovering washes. Certain results are presented below demonstrating their effectiveness. IEX resins were pre-conditioned.

Protocol IEX-V (Pre-Masking and Post-Recovering (with Ethanol))
(i) Pre-masking: Pre-conditioned resin (400 mL) was stirred in contact with aqueous 0.500 glycol compound (1.5 kg) for 30 minutes. The resin was recovered by filtration. The filtrate was analyzed (e.g., by TIPLC and/or IC). Loss of a glycol compound, e.g., THDO, during pre-masking was assessed.
(ii) The resin was stirred in contact with a simulated solution (2 L) for 1 hour. The resin was then recovered by filtration and the filtrate was analyzed (e.g., by HPLC and/or IC). Loss of a glycol compound, e.g., HDO, was assessed.
(iii) Post-recovering: The recovered resin was stirred with ethanol (2 L) for 1 hour. The resin was then recovered by filtration and the filtrate was analyzed (e.g., by HPLC and/or IC). Recovery of a glycol compound, e.g., HDO, was assessed.
   IEX-VI (Post-Recovering (with Ethanol))
(i) Pre-conditioned resin (400 mL) was stirred in contact with a simulated solution (2 L) for 1 hour. The resin was then recovered by filtration and the filtrate was analyzed (e.g., by HPLC and/or IC). Loss of a glycol compound, e.g., HDO, was assessed.
(ii) Post-recovering: The recovered resin was stirred with ethanol (2 L) for 1 hour. The resin was then recovered by filtration and the filtrate was analyzed (e.g., by HPLC and/or IC). Recovery of a glycol compound, e.g., HDO, was assessed.

As demonstrated herein, ethanol can effectively reduce glycol compound loss.

TABLE 29
Certain results for a strong cation exchange
resin, polystyrene (SK1BH) Mitsubishi DIAION

| | | Ratio of total | K⁺/ | Mg²⁺/ |
|---|---|---|---|---|
| | | HDO loss wt/% | ppm | ppm |
| Simulated | | — | 3100 | 91 |
| Solution A | | | | |
| IEX-I | — | 10.3 | 120 | 0.4 |
| IEX-II | Pre-masking, and post- | 0.431 | 123 | 0.7 |
| | recovering with water | | | |
| IEX-IV | Post-recovering with | 1.01 | 130 | 0.5 |
| | water | | | |
| IEX-V | Pre-masking, and post- | 0.419 | 121 | 0.5 |
| | recovering with ethanol | | | |
| IEX-VI | Post-recovering with | 1.09 | 128 | 0.8 |
| | ethanol | | | |

### Example 4. Provided Technologies can be Utilized in Various Application Setups

Among other things, provided technologies can be utilized in various setups and instrumentations. For example, in some embodiments, provided technologies can be utilized in column setups as described in the present Example.

IEX resin was pre-conditioned. For example, in some embodiments, for a H/OH or free type resin, about 300 mL resin was stirred and washed with a suitable amount of pure water (about 2250 g) for a suitable period of time, e.g., about 1 hour. The washed resin was soaked in pure water for another period of time, e.g., about 12 hours. After that, the soaked resin was recovered by filtration. In some embodiments, for a Cl-type resin, about 300 mL resin was stirred and regenerated to OH-type with a suitable amount of a base solution at a suitable concentration, e.g., about 10% NaOH. aq about 1500 g. The regenerated resin was stirred and washed with a suitable amount of ion-exchanged water, e.g., about 2250 g. The washed resin was soaked in pure water for a suitable period of time, e.g., about 12 hours. The soaked resin was recovered by filtration.

Certain useful protocols are described below as examples.

### Protocol IEX-I (a “Normal” Method Useful as a Reference Method for Comparison)

(i) 300 mL preconditioned resin was filled into a column, and pure water was flowed for 10 min at 20 mL/min, 25° C.
(ii) 12,350 mL simulated solution A was flowed at 20 mL/min at 25° C. and each fraction was collected.
(iii) Each collected fraction was analyzed by HPLC and IC. Breakthrough point and HDO loss were assessed.

### Protocol IEX-II (Pre-Masking+Post-Recovering)

(i) 300 mL preconditioned resin was filled into a column, and pure water was flowed for 10 min at 20 mL/min, 25° C.
(ii) 0.5% HDO.aq was flowed for 30 minutes at 20 mL/min, 25° C. (pre-masking) and each fraction was collected.
(iii) 13 L simulated solution A was flowed at 20 mL/min, 25° C. and each fraction was collected.
(iv) 2700 mL of pure water was flowed at 20 mL/min, 25° C. and each fraction was collected (post-recovering).
(v) Each collected fraction was analyzed by HPLC and IC. Breakthrough point and HDO loss were estimated.

### Protocol IEX-III (Pre-Masking Only)

(i) 300 mL preconditioned resin was filled into a column, and pure water was flowed for 10 min at 20 mL/min, 25° C.
(ii) 0.5% HDO.aq was flowed for 30 minutes at 20 mL/min, 25° C. (pre-masking) and each fraction was collected.
(iii) 13 L simulated solution A was flowed at 20 mL/min, 25° C. and each fraction was collected.
(iv) Each collected fraction was analyzed by HPLC and IC. Breakthrough point and HDO loss were estimated.

### Protocol IEX-IV (Post-Recovering Method)

(i) 300 mL preconditioned resin was filled into a column, and pure water was flowed for 10 min at 20 mL/min, 25° C.
(ii) 13 L simulated solution A was flowed at 20 mL/min, 25° C. and each fraction was collected.
(iii) 2700 mL of pure water was flowed at 20 mL/min, 25° C. and each fraction was collected (post-recovering).
(iv) Each collected fraction was analyzed by HPLC and IC. Breakthrough point and HDO loss were estimated.

In some embodiments, HPLC was utilized to quantify HDO amount, and IC to quantify ion concentrations. In some embodiments, ratios of total HDO loss were calculated as (can also be utilized for other glycol compounds):

$$Ratio of total HDO loss = \frac{Total HDO loss amount of IEX process}{HDO feed amount until breakthrough point} [ wt \% ]$$

wherein Total HDO loss amount of IEX process=HDO loss amount by IEX until breakthrough point+HDO loss amount by pre-masking (if pre-masking is performed)−HDO recovered amount by post-recovering (if post-masking is performed).

As demonstrated below, provided technologies, e.g., resins of hydrophilic base polymers, per-masking and/or post-recovery, can effectively reduce glycol compound loss.

TABLE 30
Certain results for a strong cation exchange
resin, polystyrene (SK1BH) Mitsubishi DIAION.

| | Ratio of total HDO loss/wt % | K⁺/ppm | Mg²⁺/ppm |
|---|---|---|---|
| Simulated | — | 3100 | 91 |
| Solution A | | | |
| IEX-I | 2.39 | 2.7 | 0.3 |
| IEX-II | 1.13 | 2.7 | 0.2 |
| IEX-III | 1.82 | 2.8 | 0.1 |
| IEX-IV | 1.43 | 2.3 | 0.2 |

TABLE 31
Certain results for a strong anion exchange resin,
polystyrene (SA10AOH) Mitsubishi DIAION.

| | Ratio of total | | | |
|---|---|---|---|---|
| | HDO loss/wt % | Pyruvate/ppm | SO₄²⁻/ppm | PO₄³⁻/ppm |
| Simulated | — | 3200 | 350 | 2400 |
| Solution A | | | | |
| IEX-I | 2.32 | 0.0 | 0.9 | 0.0 |
| IEX-II | 1.43 | 0.0 | 1.1 | 0.0 |
| IEX-III | 1.96 | 0.0 | 0.7 | 0.0 |
| IEX-IV | 1.67 | 0.0 | 0.8 | 0.0 |

TABLE 32
Certain results for a strong anion exchange resin,
polyacrylic (HPR4850 Cl) AmberLite DuPont.

| | Ratio of total | | | |
|---|---|---|---|---|
| | HDO loss/wt % | Pyruvate/ppm | SO₄²⁻/ppm | PO₄³⁻/ppm |
| Simulated | — | 3200 | 350 | 2400 |
| Solution A | | | | |
| IEX-I | 0.57 | 0.0 | 1.0 | 0.0 |
| IEX-II | 0.51 | 0.0 | 1.3 | 0.0 |
| IEX-III | 0.55 | 0.0 | 0.8 | 0.0 |
| IEX-IV | 0.54 | 0.0 | 1.1 | 0.0 |

TABLE 33
Certain results for a strong anion exchange resin,
polyacrylic (SCAV4 Cl) AmberLite DuPont.

| | Ratio of total | | | |
|---|---|---|---|---|
| | HDO loss/wt % | Pyruvate/ppm | SO₄²⁻/ppm | PO₄³⁻/ppm |
| Simulated | — | 3200 | 350 | 2400 |
| Solution A | | | | |
| IEX-I | 0.87 | 0.0 | 1.5 | 0.0 |
| IEX-II | 0.81 | 0.0 | 1.0 | 0.0 |
| IEX-III | 0.86 | 0.0 | 0.9 | 0.0 |
| IEX-IV | 0.85 | 0.0 | 1.4 | 0.0 |

### Example 5: Evaporation & Distillation

Various evaporation and/or distillation technologies are available and can be utilized in accordance with the present disclosure. In some embodiments, a glycol compound is generated during the process of fermentation and is subsequently purified via filtration followed by ion exchange. The solution from ion exchange is evaporated, e.g., in a multiple effect evaporation process under vacuum to a final concentration of about 30-95% (e.g., about 40-95%, 40-90%, 50%-90%, or 80%-95%; or about 30%, 40%, 50%, 60%, 70%, 80%, or 90%) wt %. In some embodiments, evaporation increases concentration. In some embodiments, evaporation removes moisture. In some embodiments, evaporation reduces the vapor load in the distillation column. In some embodiments, evaporation facilitates distillation to meet a final specification of the product (in some embodiments, depending on applications, e.g., in some embodiments, as raw material for urethane, one or more or all of purity≥99.5% (wt %), APAH≤10, water≤0.1% (wt %), CPR value≤1, acid value≤0.1, ester value≤0.5). In some embodiments, evaporation removes lower boiling organic compounds along with water with minimal loss of glycol, e.g., through controlling pressure, temperature, time, etc.

In some embodiments, rising film evaporators are utilized. For example, in some embodiments, rising film evaporators connected in series are used for evaporation of aqueous mixtures. In some embodiments, an outlet from an ion exchange column, e.g., an anion exchange column, is fed into the first effect evaporator by means of feed pump and subsequently passes through a series of evaporators (n=1, 2, 3 . . . ) till the desired level of concentration is achieved, after which it can be further purified using distillation.

Various distillation technologies may be utilized in accordance with the present disclosure. In some embodiments, a continuous distillation setup is used to further purify a crude glycol compound mixture from about 80%-95% wt % to, e.g., about 95%, 96%, 97%, 98%, 99%, or >99% wt % purity. In some embodiments, a crude glycol compound composition is fractionated to provide a desired glycol compound product (e.g. 1,6-HDO) in a continuous counter current multistage fractionator under high vacuum.

### Example 6: Preparation of High Purity Compounds

Certain useful technologies for preparing high purity compounds, e.g., 1,6-HDO, and certain high purity preparations, e.g., of 1,6-HDO are presented herein as examples. In some embodiments, a method for obtaining a compound, e.g., 1,6-hexanediol, comprises culturing a microorganism in a medium containing a raw material derived from a biomass resource, and then producing a highly pure 1,6-hexanediol by further purification. Those skilled in the art appreciate that technologies described in the present example may be combined with other technologies described herein, e.g., pre-treatment and/or post-treatment, to provide compounds with high purity and/or yields.

In some embodiments, provided technologies comprise:
step (a): filtration and first ion exchange. For example, in some embodiments, a 1,6-HDO preparation was filtered to remove microorganism and other insoluble matter. The filtered-1,6-HDO preparation was then contacted by a cationic exchange resin (DIAION SK1BH) in a batch method at 40° C. After 3 hours of contact, the resin was removed by filtration to provide 1,6-HDO composition A;
step (b): second exchange. In some embodiments, 1,-HDO composition A was contacted by an anionic exchange resin (DIAION SA10AOH) in a batch method at 40° C. After 3 hours of contact, the resin was removed by filtration to provide 1,6-HDO composition B;
step (c): distillation to remove water (and/or low boiling-point impurities). In some embodiments, water was removed from 1,6-HDO composition B by a thin-film distillation apparatus by heating at 70° C. under continuous feeding operating conditions. 1,6-HDO composition B was continuously processed until the water content was ≤200 ppm, thus providing 1,6-HDO composition C;
step (d): distillation to remove impurities with relatively higher boiling points (e.g., higher than water). In some embodiments, such impurities have lower boiling points than a desired compound, e.g., 1,6-HDO. For example, in some embodiments, 1,6-HDO composition C was continuously supplied to a distillation apparatus equipped with an Oldershaw column, the top of the column was maintained at 240° C., removing volatile contaminants and providing 1,6-HDO composition D; and/or
step (e): distillation to provide high purity compound. In some embodiments, a desired compound, e.g., 1,6-HDO is distilled out to provide a high purity preparation. For example, in some embodiments, 1,6-HDO composition D was continuously supplied to a distillation apparatus equipped with an Oldershaw column, the bottom of the column was maintained at 260° C., evaporating purified 1,6-HDO with a determined purity to be >95% w/w by HPLC and GC.

In some embodiments, a method for preparing a glycol compound, e.g., 1,6-hexanediol, comprises culturing a microorganism in a medium containing a raw material derived from a biomass resource to produce the compound, and purifying the compound, e.g., 1,6-hexanediol, by technologies described herein, e.g., filtration, one or more ion changes, one or more distillations (which removes impurities and/or enriches the compound), etc.

In some embodiments, a composition comprising, e.g., 1,6-hexanediol, is provided by culturing microorganism. In some embodiments, a composition comprises a compound together with the microorganism in a culture medium. In some embodiments, microorganism is removed from the culture medium as necessary, e.g., by filtration and/or centrifugation. Those skilled in the art appreciate that various technologies may be utilized to remove microorganism. In some embodiments, microorganism and/or solid matters are removed by membrane separation and/or centrifugation. In some embodiments, continuous centrifugation with a continuous centrifuge may be used. Examples of continuous centrifuge include cage centrifuge, disc centrifuge, nozzle centrifuge, or other similar centrifugation technologies. In some embodiments, a centrifuge technology is used alone; in some embodiments, two or more centrifuge technologies are utilized in combination.

In some embodiments, a membrane is used for separation, e.g., a membrane having a pore diameter of 10.0 μm or less may be used alone, or two or more kinds of membranes of different or same pore size may be used in combination. In some embodiments, a membrane may be in one of many types of shapes, e.g., a flat membrane, a hollow fiber membrane, a spiral membrane, a tubular membrane, or a pleated membrane. In some embodiments, multiple membranes may be employed in filtration, those membranes may comprise more than one shape. Many types of filtration may be utilized in accordance with the present disclosure, e.g., dead-end method, tangential flow method, etc.

In some embodiments, purification of a glycol preparation, e.g., aqueous 1,6-HDO, comprises ion exchange. In some embodiments, ion exchange is performed in series, wherein cation exchange and anion exchange are performed one after the other. In some embodiments, cation exchange is performed first, followed by anion exchange. In some embodiments, anion exchange is performed first, followed by cation exchange. In some embodiments, pressure is applied to the liquid contacting the ion exchange resin. In some embodiments, gravitational pressure alone is applied to aqueous glycol preparation contacting the ion exchange resin.

In some embodiments, a purification comprises distillation. In some embodiments, distillation provides removal of water, removal of a component having a boiling point lower than that of a compound, e.g., 1,6-hexanediol, and/or removal of a component having a boiling point higher than that of a compound, e.g., 1,6-hexanediol. In some embodiments, removal of water, removal of a component having a boiling point lower than that of a glycol compound, e.g., 1,6-hexanediol, and removal of a component having a boiling point higher than that of a glycol compound, e.g., 1,6-hexanediol are performed in that order.

In some embodiments, an ion exchange step comprises the following steps (a) and/or (b), in some cases, in that order.
step (a): contacting a glycol compound composition, e.g., a 1,6-hexanediol composition, e.g., after membrane separation or/and centrifugation, with a cation exchange resin to obtain a glycol compound composition A; and
step (b): contacting a glycol compound composition A” obtained in step (a) with an anion exchange resin to provide a glycol compound composition B.

In some embodiments, examples of components removed by contact with the cation exchange resin in the step (a) include metal cations and ammonium ions.

Many cation exchange resins may be utilized in accordance with the present disclosure. Examples of cation exchange resins include strong acidity and weak acidity resins. In some embodiments, a cationic exchange resin is or comprises a styrene, acrylic, or hydrogel resin, or any combination thereof. In some embodiments, a cation exchange resin is or comprises a gel type, porous type, or high-porous type system. Cation exchange resins may be in different physical forms, e.g., a powdery, spherical, fibrous, membranous, etc.

In some embodiments, examples of components removed by contact with an anion exchange resin, e.g., in a step (b) include inorganic ions, e.g., chloride ions, sulfate ions, phosphate ions, etc. and/or organic acid ions and the like.

Many anion exchange resins may be utilized in accordance with the present disclosure. Examples of anion exchange resins include strong basicity and weak basicity resins. In some embodiments, an anion exchange resin is or comprises a styrene, acrylic, or hydrogel resin, or any combination thereof. In some embodiments, an anion exchange resin is or comprises a gel type, porous type, or high-porous type system. Anion exchange resins may be in different physical forms, e.g., a powdery, spherical, fibrous, membranous, etc.

Various technologies may be utilized for contacting compositions with ion exchange resins in accordance with the present disclosure, including batch methods, column methods, etc.

In some embodiments, cation exchange is performed before anion exchange. In some embodiments, anion exchange is performed before cation exchange. In some embodiments, a composition comprising a, glycol compound, e.g., 1,6-HDO, obtained by centrifugation and/or membrane separation may be brought into contact with an anion exchange resin, followed by contact with the cation exchange resin. In some embodiments, step (a) and the step (b) may be performed simultaneously. In some embodiments, the glycol compound composition, e.g., 1,6-HDO obtained by membrane separation and/or centrifugation may be brought into contact with a cation exchange resin and an anion exchange resin at the same time.

### Distillation Process

In some embodiments, distillation is carried out through steps (c), (d) and (e), optionally in this order to obtain a composition E, e.g., of 1,6-HDO. In some embodiments, purification optionally comprises step (f):
step (c): distillation that removes water from a composition containing a glycol compound, e.g., 1,6-hexanediol In some embodiments, distillation is performed on a composition obtained after ion exchange (e.g., a glycol compound composition B) to provide a glycol compound composition C (remaining composition);
step (d): distillation that removes a component having a boiling point lower than that of a glycol compound of interest, e.g., 1,6-hexanediol. In some embodiments, distillation is performed on a composition obtained after step (c), e.g., a glycol compound composition C, to provide a glycol compound composition D (remaining composition);
step (e): distillation that removes a component that has a boiling point higher than that of a glycol compound of interest, e.g., 1,6-hexanediol. In some embodiments, distillation is performed on a composition obtained after step (d), e.g., a glycol compound composition D, to provide a glycol compound composition E (collected components from distillation); and
step (f): distillation of a composition obtained from step (e) (a glycol compound composition E) to obtain a higher purity glycol compound, e.g., 1,6-hexanediol.

In some embodiments, a component having a boiling point lower than that of a glycol of interest, e.g., in the case of 1,6-hexanediol, is, e.g., 1,3-propanediol.

In some embodiments, a component having a boiling point higher than that of a glycol of interest, e.g., in the case of 1,6-hexanediol is, e.g., glycerin.

Various distillation technologies may be utilized in accordance with the present disclosure. In some embodiments, a distillation method is continuous distillation. In some embodiments, a distillation method is batch distillation.

### Step (c)

In some embodiments, a method comprises step (c) that removes water. In some embodiments, water is removed from a glycol compound composition, e.g., a glycol compound composition B by heating a glycol compound composition, e.g., obtained in an ion exchange step to a temperature at which water evaporates. In some embodiments, pressure is reduced.

Various technologies may be utilized for step (c) in accordance with the present disclosure. Examples of useful apparatus include a continuous distillation column, a multiple utility can, a thin film evaporator, an evaporator, a batch distiller, an atomization separator, and the like.

### Step (d)

In some embodiments, a method comprises step (d) that removes a component having a boiling point lower than a desired glycol compound, e.g., that of 1,6-hexanediol. For example, in some embodiments, such a component is removed from a glycol compound composition, e.g., a glycol compound composition C (e.g., wherein the glycol compound is 1,6-HDO).

In some embodiments, step (d) removes a component having a low boiling point. In some embodiments, step (d) removes a trace amount of a component causing coloring. For example, in some embodiments, for 1,6-HDO purification, components having a boiling point lower than that of 1,6-hexanediol including certain coloring causing components, hydrogenated products of certain components causing coloring are removed or reduced.

Various technologies may be utilized for step (d) in accordance with the present disclosure. For example, distillation in step (d) can be performed utilizing means and apparatus such as atmospheric distillation, vacuum distillation, pressurized distillation, etc. in accordance with the present disclosure. Useful apparatus include continuous distillation columns, multiple utility cans, thin film evaporators, evaporators, batch distillers, atomization separators, etc. Those skilled in the art can assess and set operating conditions used in step (d), e.g., in consideration of a glycol compound composition C (e.g., wherein the glycol compound is 1,6-HDO), purity to be obtained, etc.

### Step (e)

In some embodiments, a method comprises step (e) that removes a component having a boiling point higher than that of a desired glycol compound, e.g., 1,6-hexanediol. For example, in some embodiments, such a component is removed from a glycol compound composition, e.g., a glycol compound composition D (e.g., wherein the glycol compound is 1,6-HDO).

In some embodiments, a component having a boiling point higher than that of a glycol of interest, e.g., 1,6-hexanediol, is particular to a fermentation method. In some embodiments, it is a nitrogen-containing component derived from an amino acid or a protein. In some embodiments, it is a sugar or its decomposed product.

Various technologies may be utilized for step (e) in accordance with the present disclosure. Useful apparatus include continuous distillation columns, multiple utility cans, thin film evaporators, evaporators, batch distillers, atomization separators, etc. Those skilled in the art can assess and set operating conditions used in step (e), e.g., in consideration of a glycol compound composition D (e.g., wherein the glycol compound is 1,6-HDO), purity to be obtained, etc.

### Step (f)

In some embodiments, a composition, e.g., a glycol compound composition E obtained through steps (c), (d) and (e) is further purified to obtain a higher purity composition.

Various technologies may be utilized for step (f) in accordance with the present disclosure. Useful apparatus include continuous distillation columns, multi-effect cans, thin-film evaporators, evaporators, batch distillers, atomization separators, etc. Those skilled in the art can assess and set operating conditions used in step (f), e.g., in consideration of the composition of a glycol compound to be purified, a purity to be finally obtained, etc.

In some embodiments, a glycol compound composition, e.g., a 1,6-HDO composition, is obtained by carrying out step (e) and step (f) if necessary.

In some embodiments, steps (c), (d), and (e) may be performed in that order. In some embodiments, steps (c), (d), and (e) may be performed in a different order compared to described above.

A useful process is described in FIG. 1 as an example. In some embodiments, such a process is utilized to purify 1,6-HDO. In some embodiments, filtration is microfiltration.

### Step (a): Ion Exchange to Remove Cations

In some embodiments, cations in a glycol compound composition, e.g., a microorganism-removed glycol compound composition, e.g., a 1,6-hexanediol composition, were removed. In some embodiments, they were removed by membrane separation. In some embodiments, step (a) removed or reduced levels of various cations. In some embodiments, cation exchange was performed in a batch manner. In some embodiments, a contact temperature with the cation exchange resin was about 40° C., and DIAION SK1BH manufactured by Mitsubishi Chemical Co., Ltd. was added to a glycol compound composition, e.g., a 1,6-hexanediol composition, and stirred for 3 hours. After stirring, filtration was performed to obtain a glycol compound composition, e.g., a glycol compound composition A (e.g., 1,6-HDO composition A) as a filtrate.

### Step (b): Ion Exchange to Remove Anions

In some embodiments, anions in a glycol compound (e.g., 1,6-HDO) composition, e.g., a glycol compound composition A, were removed. In some embodiments, step (b) removed or reduced levels of various anions. In some embodiments, anion exchange was performed in a batch manner. In some embodiments, a contact temperature with the anion exchange resin was about 40° C., and DIAION SA 10 AOH produced by Mitsubishi Chemical Co., Ltd. was added to a glycol compound composition, e.g., a 1,6-hexanediol composition, and stirred for 3 hours. After stirring, filtration was performed to obtain a glycol compound composition, e.g., a glycol compound composition B (e.g. 1,6-HDO composition B) as a filtrate.

### Step (c): Removing Water

In some embodiments, a step removes water. In some embodiments, step (c) comprises removal of water. In some embodiments, water is removed in an evaporation step. In some embodiments, a thin-film distiller was used as the apparatus for step (c). In some embodiments, jacket temperature was set at about 70° C., and a composition containing a glycol compound, e.g., 1,6-hexanediol, was continuously introduced to evaporate water (and other constituent of the composition for which evaporation is possible). In some embodiments, simultaneously with evaporation of water, dehydrated glycol compound composition (e.g., 1,6-HDO composition C) was continuously drawn out from the bottom of the apparatus. In some embodiments, the water concentration in a glycol compound composition C (e.g., 1,6-HDO composition C) was 0.020 mass % (200 mass ppm).

### Step (d): Distillation Separation of Low Boiling Point Components

In some embodiments, components lower in boiling point than a glycol compound of interest, e.g., 1,6-hexanediol, contained in a glycol compound composition, e.g., a glycol compound composition C (e.g., 1,6-HDO composition C), was removed by a continuous distillation column. In some embodiments, an Oldershaw distillation column was used as the distillation column in step (d). In some embodiments, a glycol compound composition obtained in in step (c) (e.g., 1,6-HDO composition C) was continuously supplied to the distillation column, and the top temperature of the column was controlled to a constant temperature of about 240° C. In some embodiments, low boiling point components were removed, and a glycol compound composition with lower levels of components with lower boiling points was obtained from the bottom of a distillation column to provide glycol compound composition D (e.g., 1,6-HDO composition D).

### Step (e): Distillation Separation of High Boiling Components

In some embodiments, components having higher boiling points than a glycol compound of interest (e.g., 1,6-hexanediol) were removed by distillation. In some embodiments, distillation is performed with a continuous distillation column. In some embodiments, an Oldershaw distillation column was used as the distillation column for step (e). In some embodiments, a glycol compound composition, (e.g., 1,6-HDO composition D) obtained in step (d), was continuously supplied to a distillation column, and the column bottom temperature was controlled to be constant at about 260° C. In some embodiments, high boiling point components in a glycol compound composition, e.g., 1,6-HDO composition D were removed by continuous extraction from the bottom of the column. In some embodiments, a glycol compound composition, e.g., 1,6-HDO composition E, is obtained as a top distillate. In some embodiments, a glycol compound composition, e.g., a 1,6-HDO composition E (top distillate) is obtained by removing a component having a boiling point higher than that of a glycol compound of interest, e.g., 1,6-hexanediol, from the top of a column.

In some embodiments, purity of a glycol compound obtained, e.g., 1,6-hexanediol, was determined by HPLC and/or GC. In some embodiments, purity was confirmed to be 95% or more, respectively.

### EQUIVALENTS

Those skilled in the art will recognize, or be able to ascertain using no more than routine experimentation, many equivalents to the specific embodiments of the invention described herein.

We claim:

1. A method of purifying an aqueous initial preparation comprising a glycol compound, comprising contacting the aqueous initial preparation with an ion exchange resin, wherein the base polymer of the resin is a hydrophilic polymer, wherein:
the glycol compound is 1,6-hexanediol; and
the ion exchange resin is pre-masked by contacting the ion exchange resin with a pre-masking preparation comprising the glycol compound prior to contacting the aqueous initial preparation with the ion exchange resin.

2. A method of purifying an aqueous initial preparation comprising a glycol compound,
wherein the glycol compound has 5 to 12 carbon atoms, and
wherein the aqueous initial preparation comprises greater than 50% water; the method comprising:
contacting the aqueous initial preparation with an ion exchange resin, wherein the resin is a hydrophilic resin; or
a method of purifying an aqueous initial preparation comprising a glycol compound, comprising contacting the aqueous initial preparation with an ion exchange resin, wherein the base polymer of the resin is a hydrophilic polymer.

3. The method of any one of the preceding claims, wherein the hydrophilic resin is a polyacrylic resin.

4. The method of claim 3, wherein the polyacrylic resin is a strong anionic exchange resin, a weak anionic exchange resin or a weak cationic exchange resin.

5. The method of claim 3, wherein the hydrophilic resin is a polymethacrylic resin.

6. The method of claim 2, further comprising contacting the ion exchange resin with a pre-masking preparation comprising the glycol compound prior to contacting the aqueous initial preparation with the ion exchange resin.

7. A method of purifying an aqueous initial preparation comprising a glycol compound, comprising contacting an ion exchange resin with a pre-masking preparation comprising the glycol compound, and contacting the aqueous initial preparation with the pre-masked ion exchange resin; or a method of purifying an aqueous initial preparation comprising a glycol compound, comprising contacting an ion exchange resin with a pre-masking preparation comprising the glycol compound, and contacting the aqueous initial preparation with the pre-masked ion exchange resin; optionally further comprising contacting the ion exchange resin with a post-recovering solvent system after contacting the resin with the aqueous initial preparation; or
a method of purifying an aqueous initial preparation comprising a glycol compound, comprising contacting an aqueous initial preparation with an ion exchange resin, and contacting the ion exchange resin with a post-recovering solvent system after contacting the aqueous initial preparation with the ion exchange resin; or
a method of purifying an aqueous initial preparation comprising a glycol compound, comprising contacting an ion exchange resin with a pre-masking preparation comprising the glycol compound, contacting an aqueous initial preparation with the pre-masked ion exchange resin, and contacting the ion exchange resin with a post-recovering solvent system after contacting the aqueous initial preparation with the pre-masked ion exchange resin.

8. The method of any one of the preceding claims, wherein the aqueous initial preparation comprises greater than about 50% water, or wherein water is present in the preparation in an amount within a range of 51% to 99%, or wherein the aqueous initial preparation comprises greater than about 70% water, or wherein the aqueous initial preparation comprises greater than about 90% water.

9. The method of any one of the preceding claims, wherein the aqueous initial preparation comprises one or more ions and/or impurities independently at about or at least about 50, 60, 70, 80, 90, 100, 150, 200, 300, 400, 500, 600, 700, 800, 900, 1000, 1500, 2000, 3000, 4000, 5000, 6000, 7000, 8000, or 10000 ppm (w/w), and/or wherein the aqueous initial preparation comprises K⁺ at about or at least about 3000 ppm (w/w), and/or or wherein the aqueous initial preparation comprises Mg²⁺ at about or at least about 90 ppm (w/w), and/or wherein the aqueous initial preparation comprises pyruvate at about or at least about 3000 ppm (w/w), and/or wherein the aqueous initial preparation comprises SO₄²⁻ at about or at least about 350 ppm (w/w), and/or wherein the aqueous initial preparation comprises phosphate (H₂PO₄⁻, HPO₄²⁻, and/or PO₄³⁻) at about or at least about 2000 ppm (w/w).

10. The method of any one of the preceding claims, comprising contacting an aqueous initial preparation with an acidic resin, and/or comprising contacting an aqueous initial preparation with a strong acidic resin, and/or comprising contacting an aqueous initial preparation with a weak acidic resin.

11. The method of any one of claims 7-10, wherein the base polymer of the resin is hydrophobic, or wherein the resin is a polystyrene resin.

12. The method of any one of claims 7-10, wherein the base polymer of the resin is hydrophilic, or wherein the resin is a polyacrylic resin, or wherein the resin is a polymethacrylic resin.

13. The method of any one of claims 1-10, comprising contacting an aqueous initial preparation with a SK1BH resin, and/or comprising contacting an aqueous initial preparation with a WK60L resin, and/or comprising contacting an aqueous initial preparation with a WK10 resin.

14. The method of any one of claims 7-13, wherein the resin is pre-conditioned.

15. The method of any one of claims 7-14, comprising contacting the ion exchange resin with a pre-masking preparation comprising the glycol compound prior to contacting the ion exchange resin with an aqueous initial preparation.

16. The method of any one of claims 7-14, comprising contacting the ion exchange resin with a post-recovering solvent system after contacting the ion exchange resin with an aqueous initial preparation.

17. The method of any one of the preceding claims, comprising contacting an aqueous initial preparation with a basic resin, and/or comprising contacting an aqueous initial preparation with a strong basic resin, and/or comprising contacting an aqueous initial preparation with a weak basic resin; optionally wherein the base polymer of the resin is hydrophobic or is a polystyrene resin, or optionally wherein the base polymer of the resin is hydrophilic, or is a polyacrylic resin or a polymethacrylic resin.

18. The method of any one of claims 1-17, comprising contacting an aqueous initial preparation with a SCA10AOH resin, and/or comprising contacting an aqueous initial preparation with a WA21J resin, and/or comprising contacting an aqueous initial preparation with a HPR4580 Cl resin, and/or comprising contacting an aqueous initial preparation with a SCAV4 Cl resin, and/or comprising contacting an aqueous initial preparation with an IRA67 resin.

19. The method of any one of 17-18, wherein the resin is pre-conditioned.

20. The method of any one of claims 17-19, comprising contacting the ion exchange resin with a pre-masking preparation comprising the glycol compound prior to contacting the ion exchange resin with an aqueous initial preparation.

21. The method of any one of claims 17-20, comprising contacting the ion exchange resin with a post-recovering solvent system after contacting the ion exchange resin with an aqueous initial preparation.

22. The method of any one of the preceding claims, wherein the resin is characterized by an average cross-linking within a range of 1% to 25%, and/or wherein the resin is characterized by an average functional group density within a range of 0.1 to 4.0 (eq/L), and/or wherein the resin is characterized by an average particle size within a range of 100 μm to 1500 μm, and/or wherein the resin is resin is characterized as type 1 or type 2.

23. The method of any one of the preceding claims, wherein concentration of the glycol compound in the pre-masking preparation is about or no more than about 50% of the glycol compound (wt %), or wherein concentration of the glycol compound in the pre-masking preparation is about or no more than about 30% of the glycol compound (wt %), or wherein concentration of the glycol compound in the pre-masking preparation is about or no more than about 20% of the glycol compound (wt %), or wherein concentration of the glycol compound in the pre-masking preparation is about or no more than about 10% of the glycol compound (wt %), or wherein concentration of the glycol compound in the pre-masking preparation is about or no more than about 5% of the glycol compound (wt %), or wherein concentration of the glycol compound in the pre-masking preparation is about or no more than about 2% of the glycol compound (wt %), or wherein concentration of the glycol compound in the pre-masking preparation is about or no more than about 0.1% to 2% of the glycol compound (wt %), or wherein concentration of the glycol compound in the pre-masking preparation is about or no more than about 1% of the glycol compound (wt %), or wherein concentration of the glycol compound in the pre-masking preparation is about or no more than about 0.3% to 0.7% of the glycol compound (wt %), or wherein concentration of the glycol compound in the pre-masking preparation is about or no more than about 0.5% of the glycol compound (wt %), or wherein concentration of the glycol compound in the pre-masking preparation is about or no more than about 0.2% of the glycol compound (wt %), or wherein concentration of the glycol compound in the pre-masking preparation is about or no more than about 0.1% of the glycol compound (wt %).

24. The method of any one of the preceding claims, wherein for each contacting of an ion exchange resin with a pre-masking preparation, about or at least about 1-1000, e.g., about 1-500, 1-200, 1-100, 1-50, 1-20, 1-10, or about 1, 2, 5, 10, 15, 20, 25, 30, 40, 50, 60, 70, 80, 90, 100, 150, 200, 300, 400, 500, 600, 700, 800, 900 or 1000 mg of the glycol compound is utilized per mL of resin.

25. The method of any one of the preceding claims, wherein for each pre-masking preparation, one or more ions or impurities independently have a lower level in the pre-masking preparation than the aqueous initial preparation, or wherein each pre-masking preparation is substantially free of one or more ions and/or impurities present in the aqueous initial preparation, or wherein each pre-masking preparation is substantially free of all ions and/or impurities present in the aqueous initial preparation, and/or wherein the solvent of the pre-masking preparation is water.

26. The method of any one of the preceding claims, wherein for each contacting of the ion exchange resin with a post-recovering solvent system, about or at least about 1 to 100, e.g., about 1-50, 1-20, 1-10, 2-10, 5-10, or about 1, 2, 3, 4, 5, 6, 7, 8, 9, 10, 15, 20, 30, 40, 50, 60, 70, 80, 90 or 100 mL of a post-recovering solvent system is independently applied per 1 mL of a resin, or wherein for each contacting of the ion exchange resin with a post-recovering solvent system, about 1-10 mL of a post-recovering solvent system is independently applied per 1 mL of a resin.

27. The method of any one of the preceding claims, wherein a post-recovering solvent system comprises water, or wherein a post-recovering solvent system comprises an alcohol, or wherein a post-recovering solvent system comprises a short-chain alcohol, or wherein a post-recovering solvent system comprises methanol, or wherein a post-recovering solvent system comprises ethanol, or wherein a post-recovering solvent system comprises an organic solvent soluble in water, or wherein a post-recovering solvent system comprises THF, or wherein a post-recovering solvent system comprises acetone, or wherein a post-recovering solvent system is water, or wherein a post-recovering solvent system is an alcohol, or wherein a post-recovering solvent system is a short-chain alcohol, or wherein a post-recovering solvent system is methanol, or wherein a post-recovering solvent system is ethanol, or wherein a post-recovering solvent system is a mixture of two or more solvent selected from water and short-chain alcohols.

28. The method of any one of the preceding claims, wherein the total ion content of each post-recovering solvent system is independently no more than about 1000, 900, 800, 700, 600, 500, 400, 300, 200, 100, 90, 80, 70, 60, 50, 40, 30, 20, 10, 9, 8, 7, 6, 5, 4, 3, 2, or 1 ppm (wt/wt), or wherein the total ion content of each post-recovering solvent system is independently no more than about 100 ppm (wt/wt), or wherein the total ion content of each post-recovering solvent system is independently no more than about 10 ppm (wt/wt), or wherein each post-recovering solvent system is substantially free of ions and compounds other than the solvent(s), or wherein for each contacting of the ion exchange resin with a post-recovering solvent system, about or at least about 1 to 100, e.g., about 1-50, 1-20, 1-10, 2-10, 5-10, or about 1, 2, 3, 4, 5, 6, 7, 8, 9, 10, 15, 20, 30, 40, 50, 60, 70, 80, 90 or 100 mL of a solvent system is applied per 1 mL of a resin (mL/mL), or wherein for each contacting of the ion exchange resin with a post-recovering solvent system, about 1-20 mL of a solvent system is applied per 1 mL of a resin (mL/mL), or wherein for each contacting of the ion exchange resin with a post-recovering solvent system, about 1-10 mL of a solvent system is applied per 1 mL of a resin (mL/mL).

29. The method of any one of the preceding claims, wherein after contacting an aqueous initial preparation with the resin(s), concentrations of one or more ions (e.g., inorganic ions (e.g., sulfate, phosphate, pyruvate, lithium, sodium, potassium, ammonium, calcium, magnesium, iron(II), iron(III), aluminum, chloride), and organic ions (e.g., acetate, lactate, malate, succinate, maleate)) are independently no more than about 1000, 900, 800, 700, 600, 500, 400, 300, 200, 100, 90, 80, 70, 60, 50, 40, 30, 20, 10, 9, 8, 7, 6, 5, 4, 3, 2, 1, 0.9, 0.8, 0.7, 0.6, 0.5, 0.4, 0.3, 0.2 or 0.1 ppm (w/w).

30. The method of any one of the preceding claims, wherein a resin is in the shape of a powder, or wherein a resin is spherical, or wherein a resin is a membrane.

31. The method of any one of the preceding claims, wherein a contact with a resin is performed in a batch method, in a column and/or in a continuous flow method.

32. The method of any one of the preceding claims, wherein after contacting an aqueous initial preparation with the resin(s), concentrations of one or more ions (e.g., inorganic ions (e.g., sulfate, phosphate, pyruvate, lithium, sodium, potassium, ammonium, calcium, magnesium, iron(II), iron(III), aluminum, chloride), and organic ions (e.g., acetate, lactate, malate, succinate, maleate)) are independently no more than about 100 ppm (w/w), and/or wherein after contacting an aqueous initial preparation with the resin(s), the concentration of K⁺ is no more than 200, 100, 50, 20, 10, 5, 1, or 0.5 ppm, and/or wherein after contacting an aqueous initial preparation with the resin(s), the amount of K⁺ is reduced at about or at least about 90% or at least about 99% compared to the aqueous initial preparation; and/or wherein after contacting an aqueous initial preparation with the resin(s).

33. The method of any one of the preceding claims, wherein the concentration of Mg²⁺ is no more than 200, 100, 50, 20, 10, 5, 1, or 0.5 ppm, or wherein after contacting an aqueous initial preparation with the resin(s), the amount of Mg²⁺ is reduced at about or at least about 90% or at least about 99% compared to the aqueous initial preparation.

34. The method of any one of the preceding claims, wherein after contacting an aqueous initial preparation with the resin(s), the concentration of pyruvate is no more than 200, 100, 50, 20, 10, 5, 1, or 0.5 ppm, and/or wherein after contacting an aqueous initial preparation with the resin(s), the amount of pyruvate is reduced at about or at least about 90% or at least about 99% compared to the aqueous initial preparation; and/or

[symbol]

35. The method of any one of the preceding claims, wherein after contacting an aqueous initial preparation with the resin(s), the concentration of SO₄²⁻ is no more than 200, 100, 50, 20, 10, 5, 1, or 0.5 ppm, and/or wherein after contacting an aqueous initial preparation with the resin(s), the amount of SO₄²⁻ is reduced at about or at least about 90% or at least about 99% compared to the aqueous initial preparation.

36. The method of any one of the preceding claims, wherein after contacting an aqueous initial preparation with the resin(s), the concentration of phosphate (H₂PO₄⁻, HPO₄²⁻, and/or PO₄³⁻) is no more than 200, 100, 50, 20, 10, 5, 1, or 0.5 ppm, and/or wherein after contacting an aqueous initial preparation with the resin(s), the amount of phosphate (H₂PO₄⁻, HPO₄²⁻, and/or PO₄³⁻) is reduced at about or at least about 90% or at least about 99% compared to the aqueous initial preparation.

37. The method of any one of the preceding claims, comprising preparing the aqueous initial preparation from fermentation, and/or comprising biomass deactivation.

38. The method of any one of the preceding claims, comprising preparing the aqueous initial preparation from fermentation, wherein the preparing comprises centrifugation, continuous centrifugation, filtration, microfiltration, nanofiltration, and/or a membrane filtration, optionally wherein the pore size is about 10 μm or less and/or wherein the filter membrane shape is flat, a hollow tube fiber, tubular, spiral, or pleated.

39. The method of any one of the preceding claims, comprising evaporating water from the preparation provided by contacting an aqueous initial preparation with an ion exchange resin, and/or comprising evaporating water from the preparation provided by contacting an aqueous initial preparation with an ion exchange resin under vacuum.

40. The method of any one of the preceding claims, further comprising distillation that removes a component that has a lower boiling point than the glycol compound, and/or comprising distilling a glycol compound from a composition comprising the glycol compound, and/or wherein the distillation is performed under vacuum.

41. The method of any one of the preceding claims, comprising polishing, and/or comprising polishing after evaporation and before distillation, optionally wherein polishing reduces levels of one or more salts, optionally wherein polishing comprises contact with a cation exchange resin, optionally wherein polishing comprises contact with an anion exchange resin, and/or optionally wherein polishing comprises contact with a porous material optionally wherein polishing comprises contact with activated carbon.

42. The method of any one of the preceding claims, comprising decolorization of the glycol compound preparation, optionally wherein decolorization comprises hydrogenation; and/or wherein decolorization comprises contact with a porous material, optionally wherein decolorization comprises contact with activated carbon.

43. The method of any one of the preceding claims, comprising deodorization of the glycol compound preparation, optionally wherein deodorization comprises hydrogenation; and/or wherein deodorization comprises contact with a porous material, optionally wherein deodorization comprises contact with activated carbon.

44. A glycol compound preparation prepared by a process comprising a method of any one of the preceding claims.

45. A method of making a solvent, acrylate, polymer, polyurethane, polyester, adhesive, plasticizer, or coating comprising:
providing a glycol compound preparation of claim 45,
converting the glycol compound into the solvent, acrylate, polymer, polyurethane, polyester, adhesive, plasticizer, or coating.

46. Use of a glycol compound preparation of claim 45 in the manufacturing of a solvent, acrylate, polymer, polyurethane, polyester, adhesive, plasticizer, or coating.

47. A method of characterizing a resin as suitable for purifying an aqueous initial preparation comprising a glycol compound, wherein:
the glycol compound has 5 to 12 carbon atoms, and
wherein the aqueous initial preparation comprises greater than 50% water;
the method comprising:
determining whether the particle size, cross-linking density, functional group identity, functional group density, and hydrophilicity of the resin is within the appropriate or desired range or having the appropriate or desired identity.

48. A method of characterizing a resin as suitable for purifying an aqueous initial preparation comprising a glycol compound, comprising utilizing the resin in a method of any one of claims 2-48 and assessing loss of a glycol compound during ion exchange purification.

49. The method of any one of the preceding claims, wherein loss of a glycol compound during ion exchange is no more than about 5%, about 4%, about 3%, about 2% or about 1%.

50. The method of any one of claims 48-49, wherein the resin is determined to be suitable.

51. The method of any one of the preceding claims, wherein loss of a glycol compound without pre-masking and pre-recovering is about or at least about 1.5, 2, 3, 4, 5, 6, 7, 8, 9, or 10 fold of the loss without pre-masking and without pre-recovering.

52. A method of characterizing a resin as suitable for purifying an aqueous initial preparation comprising a glycol compound, wherein:
the glycol compound has 5 to 12 carbon atoms, and
wherein the aqueous initial preparation comprises greater than 50% water; the method comprising:
contacting the resin with the aqueous initial preparation;
assessing glycol compound retention on the resin;
determining glycol compound retention is reduced relative to that observed under otherwise comparable conditions with a hydrophobic resin.

53. A purified preparation of a glycol compound in which the glycol compound is present in an amount that is about at least about 50%-100%, more than, or about or at least about or about 2, 3, 4, 5, 6, 7, 8, 9, 10, 11, 12, 13, 14, 15, 16, 17, 18, 19, 20, 30, 40, 50 or 100 fold of, that present in an aqueous initial preparation from which the glycol compound is purified; or
a purified preparation of a glycol compound in which pyruvate is present in an amount that is at least 10-fold less than that present in the aqueous preparation from which the glycol compound is purified; or
a purified preparation of a glycol compound in which phosphate is present in an amount that is at least 10-fold less than that present in an aqueous initial preparation from which the glycol compound is purified; or
a purified preparation of a glycol compound in which sulfate is present in an amount that is at least 10-fold less than that present in an aqueous initial preparation from which the glycol compound is purified; or
a purified preparation of a glycol compound in which potassium is present in an amount that is at least 10-fold less than that present in an aqueous initial preparation from which the glycol compound is purified; or
a purified preparation of a glycol compound in which magnesium is present in an amount that is at least 10-fold less than that present in an aqueous initial preparation from which the glycol compound is purified.

54. The method or preparation of any one of the preceding claims, wherein the glycol compound has about 5 to 12 carbon atoms; or wherein the glycol compound has the structure of L(OH)ₙ wherein L is a C₅₋₁₂ or C₅₋₈ hydrocarbon moiety and n is 2 or 3, optionally wherein n is 2 and optionally wherein L is linear or L is branched, and optionally wherein L is saturated or partially unsaturated; or wherein the glycol compound has the structure of HO(CH₂)ₘOH, wherein m is 5-12 or 5-8, or wherein the glycol compound is HO(CH₂)₅OH, HO(CH₂)₆OH, HO(CH₂)₇OH, HO(CH₂)₈OH, HO(CH₂)₉OH, HO(CH₂)₁₀OH, HO(CH₂)₁₁OH, or HO(CH₂)₁₂OH.

55. The method or preparation of any one of claims 2-54, wherein the glycol compound is HO(CH₂)₆OH.

56. The method or preparation of any one of Embodiments 1-235.
